(19) United States
(12) Patent Application Publication
(10) Pub. No.: US 2026/0014119 A1
(43) Pub. Date: Jan. 15, 2026

(54) EMOPAMIL-BINDING PROTEIN INHIBITORS AND USES THEREOF

(71) Applicant: BIOGEN MA INC., Cambridge, MA (US)

(72) Inventors: Felix Gonzalez Lopez de Turiso, San Diego, CA (US); Martin Himmelbauer, Cambridge, MA (US); John H. Jones, Framingham, MA (US); Edward Yin Shiang Lin, Ashland, MA (US); Robin Prince, Sharon, MA (US); Vatee Pattaropong, Bedford, MA (US); Zhili Xin, Cambridge, MA (US)

(21) Appl. No.: 18/994,344

(22) PCT Filed: Jul. 13, 2023
(86) PCT No.: PCT/US2023/027610

§ 371 Date: Jan. 14, 2025

Related U.S. Application Data
(60) Provisional application No. 63/389,482, filed on Jul. 15, 2022.

(51) Int. Cl. A61K 31/403; A61K 31/407; A61K 31/4155; A61K 31/4439; A61K 31/454; A61K 31/5377; C07D 401/04; C07D 405/12; C07D 405/14; C07D 413/04; C07D 487/04

(52) U.S. Cl. CPC A61K 31/403; A61K 31/407; A61K 31/4155; A61K 31/4439; A61K 31/454; A61K 31/5377; C07D 401/04; C07D 405/12; C07D 405/14; C07D 413/04; C07D 487/04

(57) ABSTRACT

Provided are compounds of the Formula (I) or pharmaceutically acceptable salts thereof, which are useful for the inhibition of EBP and in the treatment of a variety of EBP mediated conditions or diseases, such as multiple sclerosis.

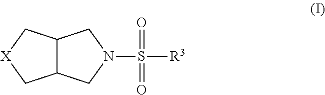

### RELATED APPLICATIONS

This application claims priority to U.S. Provisional Application No. 63/389,482 filed on Jul. 15, 2022. The entire contents of the foregoing application are expressly incorporated herein by reference.

### FIELD OF THE INVENTION

The present disclosure relates to inhibitors of Emopamil-Binding Protein (EBP), and pharmaceutically acceptable salts thereof, compositions of these compounds, processes for their preparation, their use in the treatment of diseases, their use in optional combination with a pharmaceutically acceptable carrier for the manufacture of pharmaceutical preparations, the use of the pharmaceutical preparations in the treatment of diseases, and methods of treating diseases comprising administering the EBP inhibitor to a warm-blooded animal, especially a human.

### BACKGROUND OF THE INVENTION

Emopamil-Binding Protein (EBP) is a Δ8-Δ7 sterol isomerase enzyme which isomerizes the double bond in sterol molecules, moving the double bond from the 8-9 position to the 7-8 position. Specifically, EBP converts either zymostenol to lathosterol, or zymosterol to dehydrolathosterol, during the biosynthesis of cholesterol (Silve et al., 1996, J Biol Chem. 271 (37), 22434-22440). It has been shown that an accumulation of 8-9 unsaturated sterols activates oligodendrocyte formation and remyelination (Hubler et al., 2019, Nature 560 (7718), 372-376).

Myelin is lipid-based molecule which forms protective layers (myelin sheathes) around nerve cell axons and insulates the axons. Demyelinating diseases, or myelin-related diseases, are a result of these myelin sheathes being damaged, degraded, or reduced in thickness. The loss of the myelin sheathes disrupts the electronic signals from the brain and can lead to nerve damage, vision loss, numbness, muscle weakness, cognitive decline, loss of motor functions, and other similar symptoms. In some myelin-related diseases, such as multiple sclerosis, a subject's immune system targets and breaks down their own myelin sheathes. The ability to repair and regenerate the myelin sheathes is key to treating these myelin-related diseases. Due to its function converting 8-9 sterols, inhibition of EBP is a potential target for activating remyelination, as its inhibition leads to an increase of these 8-9 sterol starting materials (Theodoropoulous et al, 2020, J Am. Chem. Soc., 142, (13), 6128-6138).

In addition to its role in remyeliniation, EBP has also been shown to be a key enzyme in certain colorectal cancers due to the reduction in essential lipids such as cholesterol (Theodoropoulous et al, 2020, J Am. Chem. Soc., 142, (13), 6128-6138).

Thus, there is a need for EBP inhibitors as potential therapeutic agents for treating diseases or disorders that are responsive to EBP inhibition.

### SUMMARY OF THE INVENTION

The present disclosure provides compounds that are EBP inhibitors. In a first embodiment, the present disclosure relates to compounds having the Formula I:

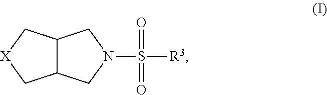

or a pharmaceutically acceptable salt thereof, wherein:
   X is NR¹ or CR^{X};
   R^{X} is —NR¹R²;
   R¹ and R² are each independently selected from H, C₁₋₆alkyl, C₄₋₈cycloalkyl, Het, or —Z-Het, wherein the C₁₋₆alkyl, C₄₋₈cycloalkyl, and Het are each optionally substituted with one or more R⁴, provided at least one of R¹ and R² is not H;
   or R¹ and R², together with the N atom from which they are attached, form a 4 to 7-membered monocyclic heterocycle or 6 to 10-membered bicyclic heterocycle, each of which is optionally substituted with one or more R⁴;
   Z is C₁₋₄alkyl;
   Het is a 4 to 6 membered monocyclic heterocyclyl, or a 6 to 8-membered bicyclic heterocyclyl, each of which is optionally substituted with one or more R⁴;
   R⁴, for each occurrence, is independently C₁₋₆alkyl or halo;
   R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
   R⁵, for each occurrence, is independently selected from halo, C₁₋₄alkyl, C₁₋₄haloalkyl, C₃₋₈cycloalkyl, —OR^{5a}, and cyano;
   R^{5a} is selected from H, C₁₋₄alkyl, and C₁₋₄haloalkyl;
   provided that the compound is not the following compound:

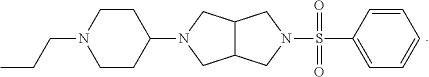 [SMILES: CCCN1CCC(N2CC3CN(S(=O)(=O)c4ccccc4)CC3C2)CC1]

Another aspect of the disclosure relates to pharmaceutical compositions comprising compounds of Formula (I) or pharmaceutically acceptable salts thereof, and a pharmaceutical carrier.

In yet another aspect, the present disclosure provides a method of treating a disease or disorder that is responsive to inhibition of EBP in a subject comprising administering to said subject an effective amount of at least one compound described herein or a pharmaceutically acceptable salt thereof. In some embodiments, the present disclosure provides a method for treating multiple sclerosis. In some embodiments, the present disclosure provides a method for promoting myelination in a subject with a myelin-related disorder.

Another aspect of the present disclosure relates to the use of at least one compound described herein or a pharmaceutically acceptable salt thereof, for the manufacture of a medicament for the treatment of a disease or disorder responsive to inhibition of EBP. Also provided is a compound described herein or a pharmaceutically acceptable salt thereof for use in treating a disease or disorder responsive to inhibition of EBP.

### DETAILED DESCRIPTION OF THE INVENTION

The present disclosure provides compounds and pharmaceutical compositions thereof that may be useful in the treatment of diseases or disorders through mediation of EBP function/activity, such as multiple sclerosis or other myelin-related disorders. In some embodiments, the compounds of present disclosure are EBP inhibitors.

### COMPOUNDS AND COMPOSITIONS

In a first embodiment, the present disclosure provides a compound of Formula (I):

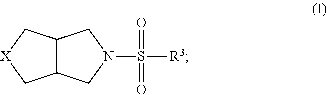

or a pharmaceutically acceptable salt thereof, wherein the variables in Formula (I) are as defined in the first embodiment above. In some embodiments, Z is C₁₋₂alkyl. In other embodiments, Z is —CH₂—.

In a second embodiment, for the compounds of Formula (I), or a pharmaceutically acceptable salt thereof, Het is a 4 to 6 membered oxygen-containing monocyclic saturated heterocyclyl or a 6 to 8-membered oxygen-containing bicyclic saturated heterocyclyl; and the remaining variables are as described in the first embodiment.

In a third embodiment, for the compounds of Formula (I), or a pharmaceutically acceptable salt thereof, R³ is phenyl, 5 or 6-membered monocyclic heteroaryl, 9 to 10 membered bicyclic heteroaryl or 8 to 10 membered bicyclic heterocycle, wherein the phenyl, 5 or 6-membered monocyclic heteroaryl, 9 to 10 membered bicyclic heteroaryl and 8 to 10 membered bicyclic heterocycle are each optionally substituted with one to three R⁵; and the remaining variables are as described in the first or second embodiment.

In a fourth embodiment, the compound of the present disclosure is represented by Formula (II):

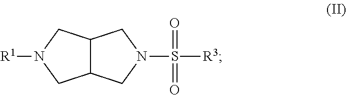

or a pharmaceutically acceptable salt thereof, wherein the variables in Formula (II) are as defined in the first, second, or third embodiment above.

In a fifth embodiment, for the compounds of Formula (II), or a pharmaceutically acceptable salt thereof, R³ is selected from the group consisting of phenyl, pyridyl, pyrimidinyl, and pyrazolyl; and the remaining variables are as described in the fourth embodiment.

In a sixth embodiment, for the compounds of Formula (II), or a pharmaceutically acceptable salt thereof, R³ is represented by the following formula:

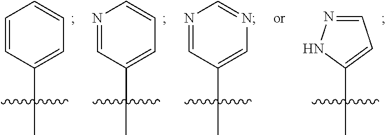

wherein each of the formula depicted above is optionally substituted with one to three R⁵; and the remaining variables are as described in the fifth embodiment.

In a seventh embodiment, for the compounds of Formula (II), or a pharmaceutically acceptable salt thereof, R³ is represented by the following formula:

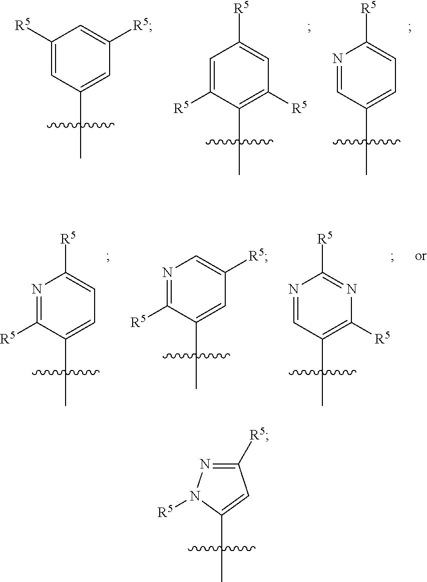

and the remaining variables are as described in the fifth embodiment. In an alternative seventh embodiment, for the compounds of Formula (II), or a pharmaceutically acceptable salt thereof, R³ is represented by the following formula:

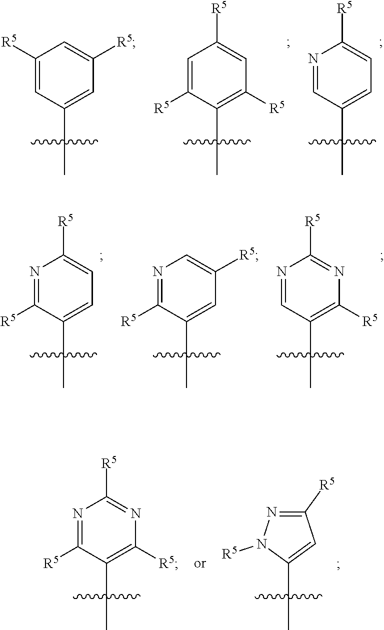

and the remaining variables are as described in the fifth embodiment.

In an eighth embodiment, for the compounds of Formula (I) or (II), or a pharmaceutically acceptable salt thereof, R⁵, for each occurrence, is independently selected from halo, C₁₋₄alkyl, C₁₋₄haloalkyl, —OR^{5a}, and —CN; and the remaining variables are as described in the first, second, third, fourth, fifth, sixth, or seventh embodiment.

In a ninth embodiment, for the compounds of Formula (I) or (II), or a pharmaceutically acceptable salt thereof, R⁵, for each occurrence, is independently selected from —Cl, —F, —CH₃, —CF₃, —OCH₃, and —CN; and the remaining variables are as described in the eighth embodiment.

In a tenth embodiment, for the compounds of Formula (I) or (II), or a pharmaceutically acceptable salt thereof, R¹ is Het; and the remaining variables are as described in the first, second, third, fourth, fifth, sixth, seventh, eighth, or ninth embodiment. In an alternative tenth embodiment, for the compounds of Formula (I) or (II), or a pharmaceutically acceptable salt thereof, R¹ is —CH₂—Het or Het; and the remaining variables are as described in the first, second, third, fourth, fifth, sixth, seventh, eighth, or ninth embodiment.

In an eleventh embodiment, for the compounds of Formula (I) or (II), or a pharmaceutically acceptable salt thereof, Het is selected from the group consisting of tetrahydropyranyl and tetrahydrofuranyl; and the remaining variables are as described in the tenth embodiment. In an alternative eleventh embodiment, for the compounds of Formula (I) or (II), or a pharmaceutically acceptable salt thereof, Het is selected from the group consisting of oxetanyl, 2-oxaspiro[3.3]heptanyl, tetrahydropyranyl and 2-oxabicyclo[2.1.1]hexanyl, 2-oxabicyclo[3.1.1]heptanyl, tetrahydrofuranyl; and the remaining variables are as described in the tenth embodiment.

In a twelfth embodiment, for the compounds of Formula (I) or (II), or a pharmaceutically acceptable salt thereof, Het is represented by the following formula:

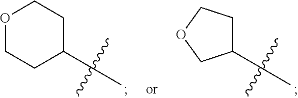 [SMILES: *C1CCOCC1 ; *C1CCOC1]

wherein each of the formula depicted above is optionally substituted with one or two R⁴; and the remaining variables are as described in the eleventh embodiment.

In some embodiments, R⁴ for each occurrence, is independently C₁₋₂alkyl. In other embodiments, R⁴ is —CH₃. In an alternative twelfth embodiment, for the compounds of Formula (I) or (II), or a pharmaceutically acceptable salt thereof, Het is represented by the following formula:

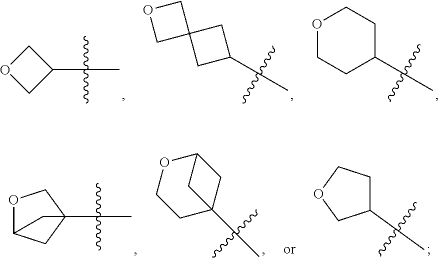 [SMILES: *C1COC1 ; *C1CC2(COC2)C1 ; *C1CCOCC1 ; *C12COC(C1)C2 ; *C12CCOC(C1)C2 ; *C1CCOC1]

wherein each of the formula depicted above is optionally substituted with one or two R⁴; and the remaining variables are as described in the eleventh embodiment. In some embodiments, R⁴ for each occurrence, is independently C₁₋₂alkyl. In other embodiments, R⁴ is —CH₃.

In a thirteenth embodiment, for the compounds of Formula (I) or (II), or a pharmaceutically acceptable salt thereof, Het is represented by the following formula:

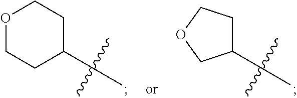 [SMILES: *C1CCOCC1 ; *C1CCOC1]

and the remaining variables are as described in the eleventh embodiment. In an alternative thirteenth embodiment, for the compounds of Formula (I) or (II), or a pharmaceutically acceptable salt thereof, Het is represented by the following formula:

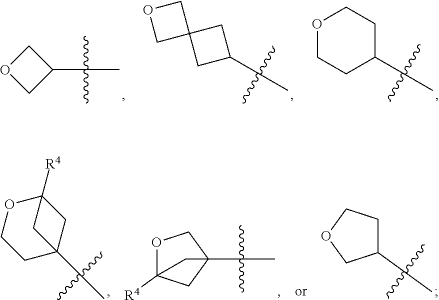

and the remaining variables are as described in the eleventh embodiment.

In a fourteenth embodiment, the compound of the present disclosure is represented by Formula (III):

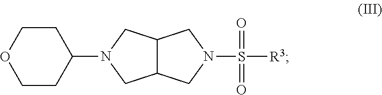

or a pharmaceutically acceptable salt thereof, wherein the variables in Formula (III) are as defined in the first, second, third, fourth, fifth, sixth, seventh, eighth, ninth, tenth, eleventh, twelfth, or thirteenth embodiment.

In a fifteenth embodiment, for the compounds of Formula (III), or a pharmaceutically acceptable salt thereof, R³ is represented by the following formula:

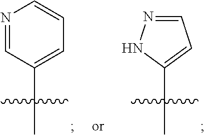 [SMILES: *c1cccnc1 ; *c1ccn[nH]1]

wherein each of the formula depicted above is optionally substituted with one to three R⁵; and the remaining variables are as described in the fourteenth embodiment.

In a sixteenth embodiment, for the compounds of Formula (III), or a pharmaceutically acceptable salt thereof, R³ is represented by the following formula:

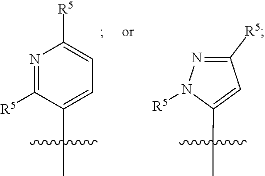

and the remaining variables are as described in the fourteenth embodiment.

In a seventeenth embodiment, for the compounds of Formula (I), (II), or (III), or a pharmaceutically acceptable salt thereof, R⁵, for each occurrence, is independently selected from C₁₋₄alkyl and C₁₋₄haloalkyl; and the remaining variables are as described in the first, second, third, fourth, fifth, sixth, seventh, eighth, ninth, tenth, eleventh, twelfth, thirteenth, fourteenth, fifteenth, or sixteenth embodiment.

In an eighteenth embodiment, for the compounds of Formula (I), (II), or (III), or a pharmaceutically acceptable salt thereof, R⁵, for each occurrence, is independently selected from —CH₃, - and —CF₃; and the remaining variables are as described in the seventeenth embodiment.

In a nineteenth embodiment, the compound of the present disclosure is represented by Formula (IV):

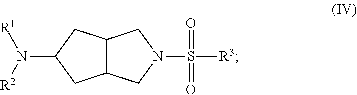

or a pharmaceutically acceptable salt thereof, wherein the variables in Formula (IV) are as defined in the first, second, or third embodiment above.

In a twentieth embodiment, for the compounds of Formula (IV), or a pharmaceutically acceptable salt thereof, R³ is selected from the group consisting of phenyl, pyridyl, and pyrazolyl; and the remaining variables are as described in the nineteenth embodiment.

In a twenty-first embodiment, for the compounds of Formula (IV), or a pharmaceutically acceptable salt thereof, R³ is represented by the following formula:

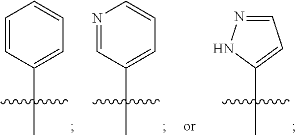

wherein each of the formula depicted above is optionally substituted with one to three R⁵; and the remaining variables are as described in the twentieth embodiment.

In a twenty-second embodiment, for the compounds of Formula (IV), or a pharmaceutically acceptable salt thereof, R³ is represented by the following formula:

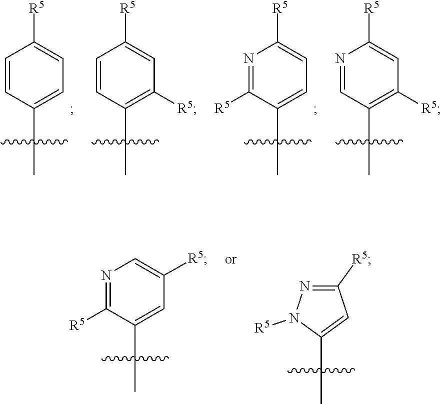

and the remaining variables are as described in the twentieth embodiment.

In a twenty-third embodiment, for the compounds of Formula (I) or (IV), or a pharmaceutically acceptable salt thereof, R⁵, for each occurrence, is independently selected from C₁₋₄alkyl, C₁₋₄haloalkyl, —OR^{5a}, and C₃₋₈cycloalkyl; and the remaining variables are as described in the first, second, third, nineteenth, twentieth, twenty-first, or twenty-second embodiment. In some embodiments, R^{5a} is C₁₋₃alkyl or C₁₋₃haloalkyl. In other embodiments, R^{5a} is C₁₋₂alkyl or C₁₋₂haloalkyl.

In a twenty-fourth embodiment, for the compounds of Formula (I) or (IV), or a pharmaceutically acceptable salt thereof, R⁵, for each occurrence, is independently selected from —CH₃, —CF₃, —OCH₃, —OCHF₂, and cyclopropyl; and the remaining variables are as described in the twenty-second embodiment.

In a twenty-fifth embodiment, for the compounds of Formula (I) or (IV), or a pharmaceutically acceptable salt thereof, R¹ is Het or —Z-Het; and R² is H or C₁₋₆alkyl; and the remaining variables are as described in the first, second, third, nineteenth, twentieth, twenty-first, twenty-second, twenty-third, or twenty-fourth embodiment. In some embodiments, Z is C₁₋₂alkyl. In other embodiments, Z is —CH₂—.

In a twenty-sixth embodiment, for the compounds of Formula (I) or (IV), or a pharmaceutically acceptable salt thereof, Het is selected from the group consisting of tetrahydropyranyl, tetrahydrofuranyl, and 2-oxaspiro[3.3]heptanyl; and the remaining variables are as described in the twenty-fifth embodiment.

In a twenty-seventh embodiment, for the compounds of Formula (I) or (IV), or a pharmaceutically acceptable salt thereof, R¹ is represented by the following formula:

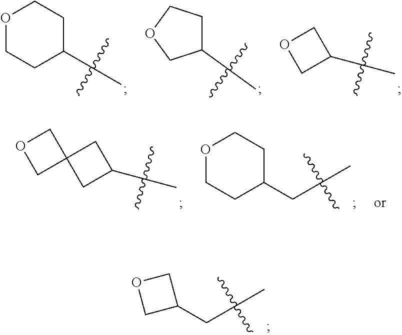 [SMILES: *C1CCOCC1 ; *C1CCOC1 ; *C1COC1 ; *C1CC2(COC2)C1 ; *CC1CCOCC1 ; *CC1COC1]

wherein each of the formula depicted above is optionally substituted with one or two R⁴; and the remaining variables are as described in the twenty-sixth embodiment. In some embodiments, R⁴ for each occurrence, is independently C₁₋₂alkyl.

In a twenty-eighth embodiment, for the compounds of Formula (I) or (IV), or a pharmaceutically acceptable salt thereof, R¹ is represented by the following formula:

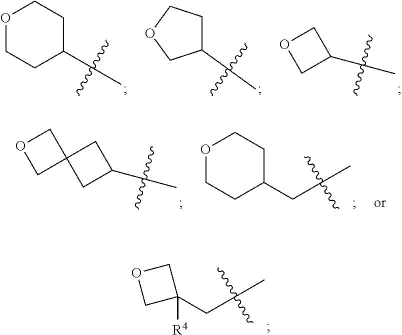

and the remaining variables are as described in the twenty-sixth embodiment. In some embodiments, R⁴ for each occurrence, is independently C₁₋₂alkyl.

In a twenty-ninth embodiment, for the compounds of Formula (I) or (IV), or a pharmaceutically acceptable salt thereof, R² is H or —CH₃; and the remaining variables are as described in the first, second, third, nineteenth, twentieth, twenty-first, twenty-second, twenty-third, twenty-fourth, twenty-fifth, twenty-sixth, twenty-seventh, or twenty-eighth embodiment.

In a thirtieth embodiment, for the compounds of Formula (I) or (IV), or a pharmaceutically acceptable salt thereof, R¹ and R², together with the N atom from which they are attached, form a 4 to 7-membered monocyclic heterocycle optionally substituted with one or more R⁴; and the remaining variables are as described in the first, second, third, nineteenth, twentieth, twenty-first, twenty-second, twenty-third, or twenty-fourth embodiment.

In a thirty-first embodiment, for the compounds of Formula (I) or (IV), or a pharmaceutically acceptable salt thereof, R¹ and R², together with the N atom from which they are attached are selected from the group consisting of piperdinyl and morpholinyl; and the remaining variables are as described in the thirtieth embodiment.

In a thirty-second embodiment, for the compounds of Formula (I) or (IV), or a pharmaceutically acceptable salt thereof, R¹ and R², together with the N atom from which they are attached are represented by the following formula:

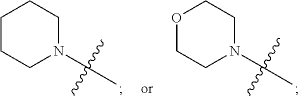 [SMILES: *N1CCCCC1 ; *N1CCOCC1]

wherein each of the formula depicted above is optionally substituted with one or two R⁴; and the remaining variables are as described in the thirty-first embodiment.

In a thirty-third embodiment, for the compounds of Formula (I) or (IV), or a pharmaceutically acceptable salt thereof, R¹ and R², together with the N atom from which they are attached are represented by the following formula

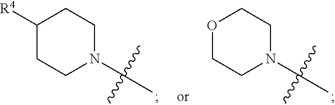

and the remaining variables are as described in the thirty-first embodiment.

In a thirty-fourth embodiment, for the compounds of Formula (IV), or a pharmaceutically acceptable salt thereof, R⁴ is —CH₃; and the remaining variables are as described in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first, thirty-second, or thirty-third embodiment.

In a thirty-fifth embodiment, the compound of the present disclosure is represented by Formula (II):

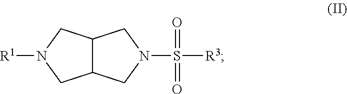

or a pharmaceutically acceptable salt thereof, wherein:
R¹ is Het, or —CH₂—Het
Het is a 4 to 6-membered monocyclic heterocyclyl, or a 6 to 8-membered bicyclic heterocyclyl, each of which is optionally substituted with one or more R⁴;
each R⁴ is independently C₁₋₃alkyl;
R³ is 5 or 6-membered monocyclic heteroaryl substituted with one or more R⁵;
each R⁵ is independently C₁₋₃alkyl and C₁₋₃haloalkyl, wherein the remaining variables in Formula (II) are as defined in the first embodiment above.

In a thirty-sixth embodiment, for the compounds of Formula (II), or a pharmaceutically acceptable salt thereof, R³ is pyridinyl, pyrimidinyl, or pyrazoyl, and the remaining variables are as described in the thirty-fifth embodiment.

In a thirty-seventh embodiment, for the compounds of Formula (II), or a pharmaceutically acceptable salt thereof, R³ is represented by the following formula:

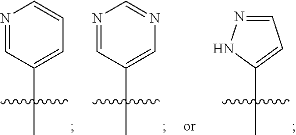

wherein each of the formula depicted above is optionally substituted with two or three R⁵; and the remaining variables are as described in the thirty-sixth embodiment.

In a thirty-eighth embodiment, for the compounds of Formula (II), or a pharmaceutically acceptable salt thereof, R³ is represented by the following formula:

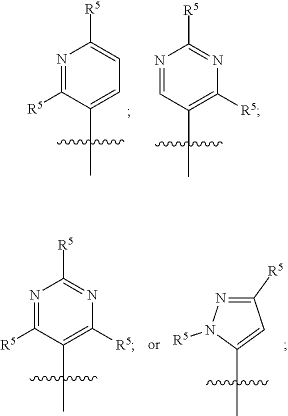

and the remaining variables are as described in the thirty-sixth embodiment.

In a thirty-ninth embodiment, for the compounds of Formula (II), or a pharmaceutically acceptable salt thereof, each R⁵ is selected from —CF₃ and —CH₃; and the remaining variables are as described in the thirty-fifth, thirty-sixth, thirty-seventh, or thirty-eighth embodiment.

In a fortieth embodiment, for the compounds of Formula (II), or a pharmaceutically acceptable salt thereof, Het is tetrohydropyranyl or 2-oxabicyclo[2.1.1]hexanyl; and the remaining variables are as described in the thirty-fifth, thirty-sixth, thirty-seventh, thirty-eighth, or thirty-ninth embodiment.

In a forty-first embodiment, for the compounds of Formula (II), or a pharmaceutically acceptable salt thereof, Het is represented by the following formula:

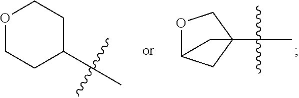 [SMILES: *C1CCOCC1 ; *C12COC(C1)C2]

wherein each of the formula depicted above is optionally substituted with one or two R⁴; and the remaining variables are as described in the fortieth embodiment.

In a forty-second embodiment, for the compounds of Formula (II), or a pharmaceutically acceptable salt thereof, Het is represented by the following formula:

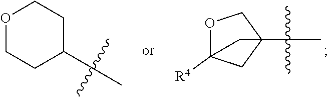

and the remaining variables are as described in the fortieth embodiment.

In a forty-third embodiment, for the compounds of Formula (II), or a pharmaceutically acceptable salt thereof, R¹ is represented by the following formula:

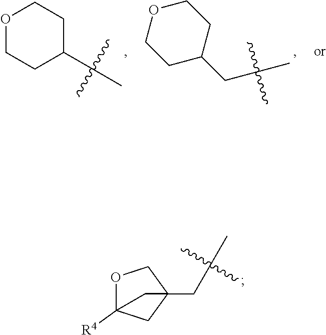

and the remaining variables are as described in the thirty-fifth, thirty-sixth, thirty-seventh, thirty-eighth, or thirty-ninth embodiment.

In a forty-fourth embodiment, for the compounds of Formula (II), or a pharmaceutically acceptable salt thereof, R⁴ is —CH₃; and the remaining variables are as described in the thirty-fifth, thirty-sixth, thirty-seventh, thirty-eighth, thirty-ninth, fortieth, forty-first, forty-second, or forty-third embodiment.

In a forty-fifth embodiment, the present disclosure provides a compound described herein (e.g., a compound of any one of Examples 1 to 62), or a pharmaceutically acceptable salt thereof.

In a forty-sixth embodiment, the present disclosure provides a compound selected from the group consisting of:
(3aR,5r,6aS)-2-((2,4-Dimethylphenyl)sulfonyl)-N-((tetrahydro-2H-pyran-4-yl)methyl)octahydrocyclopenta[c]pyrrol-5-amine;
(3aR,5r,6aS)-2-((4-(Difluoromethoxy)phenyl)sulfonyl)-N-((tetrahydro-2H-pyran-4-yl)methyl)octahydrocyclopenta[c]pyrrol-5-amine;
(3aR,5s,6aS)-2-((2,4-Dimethylphenyl)sulfonyl)-5-(4-methylpiperidin-1-yl)octahydrocyclopenta[c]pyrrole;
(3aR,5s,6aS)-2-((4-(Difluoromethoxy)phenyl)sulfonyl)-5-(4-methylpiperidin-1-yl)octahydrocyclopenta[c]pyrrole;
(3aR,5r,6aS)-2-((2,4-Dimethylphenyl)sulfonyl)-5-(4-methylpiperidin-1-yl)octahydrocyclopenta[c]pyrrole;
(3aR,5r,6aS)-2-((4-(Difluoromethoxy)phenyl)sulfonyl)-5-(4-methylpiperidin-1-yl)octahydrocyclopenta[c]pyrrole;
(3aR,5s,6aS)-2-((2,4-Dimethylphenyl)sulfonyl)-N-((tetrahydro-2H-pyran-4-yl)methyl)octahydrocyclopenta[c]pyrrol-5-amine;
(3aR,5s,6aS)-2-((4-(Difluoromethoxy)phenyl)sulfonyl)-N-((tetrahydro-2H-pyran-4-yl)methyl)octahydrocyclopenta[c]pyrrol-5-amine;
(3aR,5s,6aS)-2-((2-Methoxy-5-methylpyridin-3-yl)sulfonyl)-N-((tetrahydro-2H-pyran-4-yl)methyl)octahydrocyclopenta[c]pyrrol-5-amine;
(3aR,5s,6aS)-2-((4,6-Dimethylpyridin-3-yl)sulfonyl)-N-((tetrahydro-2H-pyran-4-yl)methyl)octahydrocyclopenta[c]pyrrol-5-amine;
(3aR,5s,6aS)-2-((1,3-Dimethyl-1H-pyrazol-5-yl)sulfonyl)-N-((tetrahydro-2H-pyran-4-yl)methyl)octahydrocyclopenta[c]pyrrol-5-amine;
(3aR,5s,6aS)-2-((3-Cyclopropyl-1-methyl-1H-pyrazol-5-yl)sulfonyl)-N-((tetrahydro-2H-pyran-4-yl)methyl)octahydrocyclopenta[c]pyrrol-5-amine;
4-((3aR,5r,6aS)-2-((4-(Difluoromethoxy)phenyl)sulfonyl)octahydrocyclopenta[c]pyrrol-5-yl)morpholine;
(3aR,5s,6aS)-2-((4-(Difluoromethoxy)phenyl)sulfonyl)-N-(oxetan-3-yl)octahydrocyclopenta[c]pyrrol-5-amine;
(3aR,5s,6aS)-2-((4-(Difluoromethoxy)phenyl)sulfonyl)-N-(tetrahydrofuran-3-yl)octahydrocyclopenta[c]pyrrol-5-amine;
(3aR,5s,6aS)-2-((4-(Difluoromethoxy)phenyl)sulfonyl)-N-(tetrahydro-2H-pyran-4-yl)octahydrocyclopenta[c]pyrrol-5-amine;
(3aR,5s,6aS)-2-((4-(Difluoromethoxy)phenyl)sulfonyl)-N-methyl-N-((tetrahydro-2H-pyran-4-yl)methyl)octahydrocyclopenta[c]pyrrol-5-amine;
(3aR,5s,6aS)-2-((3-Cyclopropyl-1-methyl-1H-pyrazol-5-yl)sulfonyl)-N-methyl-N-((tetrahydro-2H-pyran-4-yl)methyl)octahydrocyclopenta[c]pyrrol-5-amine;
(3aR,5s,6aS)-2-((4-(Difluoromethoxy)phenyl)sulfonyl)-N-(2-oxaspiro[3.3]heptan-6-yl)octahydrocyclopenta[c]pyrrol-5-amine;
(3aR,5s,6aS)-2-((3-Cyclopropyl-1-methyl-1H-pyrazol-5-yl)sulfonyl)-N-(2-oxaspiro[3.3]heptan-6-yl)octahydrocyclopenta[c]pyrrol-5-amine;
(3aR,5s,6aS)-2-((3-Cyclopropyl-1-methyl-1H-pyrazol-5-yl)sulfonyl)-N-((3-methyloxetan-3-yl)methyl)octahydrocyclopenta[c]pyrrol-5-amine;
(3aR,5s,6aS)-2-((3-Cyclopropyl-1-methyl-1H-pyrazol-5-yl)sulfonyl)-N-(tetrahydro-2H-pyran-4-yl)octahydrocyclopenta[c]pyrrol-5-amine;
(3aR,5s,6aS)-N-Methyl-2-((2-methyl-6-(trifluoromethyl)pyridin-3-yl)sulfonyl)-N-((tetrahydro-2H-pyran-4-yl)methyl)octahydrocyclopenta[c]pyrrol-5-amine;
(3aR,5s,6aS)-N-Methyl-2-((2-methyl-6-(trifluoromethyl)pyridin-3-yl)sulfonyl)-N-(tetrahydro-2H-pyran-4-yl)octahydrocyclopenta[c]pyrrol-5-amine;
(3aR,5s,6aS)-N-Methyl-2-((2-methyl-6-(trifluoromethyl)pyridin-3-yl)sulfonyl)-N-(2-oxaspiro[3.3]heptan-6-yl)octahydrocyclopenta[c]pyrrol-5-amine;
(3aR,5s,6aS)-N-Methyl-2-((2-methyl-6-(trifluoromethyl)pyridin-3-yl)sulfonyl)-N-((3-methyloxetan-3-yl)methyl)octahydrocyclopenta[c]pyrrol-5-amine;
(3aR,5s,6aS)-2-((2-Methyl-6-(trifluoromethyl)pyridin-3-yl)sulfonyl)-N-((tetrahydro-2H-pyran-4-yl)methyl)octahydrocyclopenta[c]pyrrol-5-amine;
(3aR,5s,6aS)-2-((2-Methyl-6-(trifluoromethyl)pyridin-3-yl)sulfonyl)-N-(tetrahydro-2H-pyran-4-yl)octahydrocyclopenta[c]pyrrol-5-amine;
(3aR,5s,6aS)-2-((2-Methyl-6-(trifluoromethyl)pyridin-3-yl)sulfonyl)-N-((3-methyloxetan-3-yl)methyl)octahydrocyclopenta[c]pyrrol-5-amine;
(3aR,5s,6aS)-2-((2-Methyl-6-(trifluoromethyl)pyridin-3-yl)sulfonyl)-N-(2-oxaspiro[3.3]heptan-6-yl)octahydrocyclopenta[c]pyrrol-5-amine;
(3aR,6aS)-2-((1,3-dimethyl-1H-pyrazol-5-yl)sulfonyl)-5-(tetrahydro-2H-pyran-4-yl)octahydropyrrolo[3,4-c]pyrrole;
2-(Mesitylsulfonyl)-5-(tetrahydro-2H-pyran-4-yl)octahydropyrrolo[3,4-c]pyrrole;
3-Fluoro-5-(((3aR,6aS)-5-(tetrahydro-2H-pyran-4-yl)hexahydropyrrolo[3,4-c]pyrrol-2(1H)-yl)sulfonyl)benzonitrile;
(3aR,6aS)-2-((6-Methoxy-2-methylpyridin-3-yl)sulfonyl)-5-(tetrahydro-2H-pyran-4-yl)octahydropyrrolo[3,4-c]pyrrole;
(3aR,6aS)-2-((2-Methyl-6-(trifluoromethyl)pyridin-3-yl)sulfonyl)-5-(tetrahydro-2H-pyran-4-yl)octahydropyrrolo[3,4-c]pyrrole;
(3aR,6aS)-2-((1-Methyl-3-(trifluoromethyl)-1H-pyrazol-5-yl)sulfonyl)-5-(tetrahydro-2H-pyran-4-yl)octahydropyrrolo[3,4-c]pyrrole;
(3aR,6aS)-2-((4-Methyl-2-(trifluoromethyl)pyrimidin-5-yl)sulfonyl)-5-(tetrahydro-2H-pyran-4-yl)octahydropyrrolo[3,4-c]pyrrole;
(3aR,6aS)-2-((2-Chloro-6-methoxypyridin-3-yl)sulfonyl)-5-(tetrahydro-2H-pyran-4-yl)octahydropyrrolo[3,4-c]pyrrole;
(3aR,6aS)-2-((3,5-Difluorophenyl)sulfonyl)-5-(tetrahydro-2H-pyran-4-yl)octahydropyrrolo[3,4-c]pyrrole;
(3aR,6aS)-2-((6-Methoxypyridin-3-yl)sulfonyl)-5-(tetrahydro-2H-pyran-4-yl)octahydropyrrolo[3,4-c]pyrrole;
(3aR,6aS)-2-((5-Chloro-2-methoxypyridin-3-yl)sulfonyl)-5-(tetrahydro-2H-pyran-4-yl)octahydropyrrolo[3,4-c]pyrrole;
(3aR,6aS)-2-((2-Methoxy-5-methylpyridin-3-yl)sulfonyl)-5-(tetrahydro-2H-pyran-4-yl)octahydropyrrolo[3,4-c]pyrrole;
(3aR,6aS)-2-((2,4-Dimethylpyrimidin-5-yl)sulfonyl)-5-(tetrahydro-2H-pyran-4-yl)octahydropyrrolo[3,4-c]pyrrole;
(3aR,6aS)-2-((2,4-Dimethylpyrimidin-5-yl)sulfonyl)-5-(tetrahydrofuran-3-yl)octahydropyrrolo[3,4-c]pyrrole;
(3aR,6aR)-2-((2-Methyl-6-(trifluoromethyl)pyridin-3-yl)sulfonyl)-5-(tetrahydro-2H-pyran-4-yl)octahydropyrrolo[3,4-c]pyrrole;
(3aR,6aR)-2-((1-Methyl-3-(trifluoromethyl)-1H-pyrazol-5-yl)sulfonyl)-5-(tetrahydro-2H-pyran-4-yl)octahydropyrrolo[3,4-c]pyrrole;
(3aS,6aS)-5-[4,6-dimethyl-2-(trifluoromethyl)pyrimidin-5-yl]sulfonyl-2-(oxan-4-ylmethyl)-1,3,3a,4,6,6a-hexahydropyrrolo[3,4-c]pyrrole;
(3aS,6aS)-5-[4,6-dimethyl-2-(trifluoromethyl)pyrimidin-5-yl]sulfonyl-2-(2-oxaspiro[3.3]heptan-6-yl)-1,3,3a,4,6,6a-hexahydropyrrolo[3,4-c]pyrrole;
(3aS,6aS)-5-[4,6-dimethyl-2-(trifluoromethyl)pyrimidin-5-yl]sulfonyl-2-(oxan-4-yl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(2-oxaspiro[3.3]heptan-6-yl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(oxan-4-ylmethyl)-1,3,3a,4,6,6a-hexahydropyrrolo[3,4-c]pyrrole;
rac-(3aR,6aR)-5-[4-methyl-2-(trifluoromethyl)pyrimidin-5-yl]sulfonyl-2-(2-oxaspiro[3.3]heptan-6-yl)-1,3,3a,4,6,6a-hexahydropyrrolo[3,4-c]pyrrole;
rac-(3aR,6aR)-5-[4-methyl-2-(trifluoromethyl)pyrimidin-5-yl]sulfonyl-2-(oxan-4-ylmethyl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(oxan-4-yl)-1,3,3a,4,6,6a-hexahydropyrrolo[3,4-c]pyrrole;
(3aS,6aS)-2-[(1-methyl-2-oxabicyclo[2.1.1]hexan-4-yl)methyl]-5-[2-methyl-6-(trifluoromethyl)pyridin-3-yl]sulfonyl-1,3,3a,4,6,6a-hexahydropyrrolo[3,4-c]pyrrole;
rel-(3aR,6aR)-2-[(1-methyl-2-oxabicyclo[3.1.1]heptan-5-yl)methyl]-5-[2-methyl-6-(trifluoromethyl)pyridin-3-yl]sulfonyl-1,3,3a,4,6,6a-hexahydropyrrolo[3,4-c]pyrrole;
(3aR,6aR)-2-[(1-methyl-2-oxabicyclo[2.1.1]hexan-4-yl)methyl]-5-[2-methyl-6-(trifluoromethyl)pyridin-3-yl]sulfonyl-1,3,3a,4,6,6a-hexahydropyrrolo[3,4-c]pyrrole;
(3aS,6aS)-2-[(1-methyl-2-oxabicyclo[2.1.1]hexan-4-yl)methyl]-5-[2-methyl-6-(trifluoromethyl)pyridin-3-yl]sulfonyl-1,3,3a,4,6,6a-hexahydropyrrolo[3,4-c]pyrrole;
rac-(3aR,6aR)-5-[4-methyl-2-(trifluoromethyl)pyrimidin-5-yl]sulfonyl-2-(oxan-4-yl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(oxan-4-yl)-1,3,3a,4,6,6a-hexahydropyrrolo[3,4-c]pyrrole;
(3aR,6aRS)-5-(2,5-dimethylpyrazol-3-yl)sulfonyl-2-(oxan-4-yl)-1,3,3a,4,6,6a-hexahydropyrrolo[3,4-c]pyrrole; and
rac-(3aR,6aR)-5-[2-methyl-6-(trifluoromethyl)pyridin-3-yl]sulfonyl-2-(oxetan-3-ylmethyl)-1,3,3a,4,6,6a-hexahydropyrrolo[3,4-c]pyrrole;
   or a pharmaceutically acceptable salt thereof.

In a forty-seventh embodiment, the present disclosure provides a pharmaceutical composition comprising a compound according to any one of the preceding embodiments, or a pharmaceutically acceptable salt thereof.

In a forty-eighth embodiment, the present disclosure provides a method of treating a disease or disorder mediated by EBP comprising administering to a subject an effective amount of a compound according to any one of embodiments one to thirty-six, or a pharmaceutically acceptable salt thereof, or the pharmaceutical composition of the thirty-seventh embodiment.

In a forty-ninth embodiment, the present disclosure provides a compound according to any one of embodiments one to thirty-six, for use in the treatment of a disease or disorder mediated by EBP.

In a fiftieth embodiment, the present disclosure provides the use of a compound according to any one of embodiments one to thirty-six in the manufacture of a medicament for the treatment of a disease or disorder mediated by EBP.

The compounds and intermediates described herein may be isolated and used as the compound per se. Alternatively, when a moiety is present that is capable of forming a salt, the compound or intermediate may be isolated and used as its corresponding salt. As used herein, the terms “salt” or “salts” refers to an acid addition or base addition salt of a compound described herein. “Salts” include in particular “pharmaceutical acceptable salts”. The term “pharmaceutically acceptable salts” refers to salts that retain the biological effectiveness and properties of the compounds described herein and, which typically are not biologically or otherwise undesirable. In many cases, the compounds of the present disclosure are capable of forming acid and/or base salts by virtue of the presence of amino and/or carboxyl groups or groups similar thereto.

Pharmaceutically acceptable acid addition salts can be formed with inorganic acids or organic acids, e.g., acetate, aspartate, benzoate, besylate, bromide/hydrobromide, bicarbonate/carbonate, bisulfate/sulfate, camphorsulfornate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The salts can be synthesized by conventional chemical methods from a compound containing a basic or acidic moiety. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Isotopically-labeled compounds of Formula (I) can generally be prepared by conventional techniques known to those skilled in the art or by processes analogous to those described in the accompanying Examples and Preparations using an appropriate isotopically-labeled reagents in place of the non-labeled reagent previously employed. In one embodiment, the present disclosure provides deuterated compounds described herein or a pharmaceutically acceptable salt thereof.

Pharmaceutically acceptable solvates in accordance with the invention include those wherein the solvent of crystallization may be isotopically substituted, e.g. D₂O, d₆-acetone, d₆-DMSO.

It will be recognized by those skilled in the art that the compounds of the present invention may contain chiral centers and as such may exist in different stereoisomeric forms.

As used herein, the term “an optical isomer” or “a stereoisomer” refers to any of the various stereo isomeric configurations which may exist for a given compound of the present disclosure.

It is understood that a substituent may be attached at a chiral center of a carbon atom. Therefore, the disclosure includes enantiomers, diastereomers or racemates of the compound.

“Enantiomers” are a pair of stereoisomers that are non-superimposable mirror images of each other. A 1:1 mixture of a pair of enantiomers is a “racemic” mixture. The term “racemic” or “rac” is used to designate a racemic mixture where appropriate. When designating the stereochemistry for the compounds of the present invention, a single stereoisomer with known relative and absolute configuration of the two chiral centers is designated using the conventional RS system (e.g., (1S,2S)). “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Certain of the compounds described herein contain one or more asymmetric centers or axes and may thus give rise to enantiomers, diastereomers, and other stereoisomeric forms that may be defined, in terms of absolute stereochemistry, as (R)- or (S)-.

Unless specified otherwise, the compounds of the present disclosure are meant to include all such possible stereoisomers, including racemic mixtures, optically pure forms and intermediate mixtures. Optically active (R)- and (S)-stereoisomers may be prepared using chiral synthons or chiral reagents, or resolved using conventional techniques (e.g., separated on chiral SFC or HPLC chromatography columns, such as CHIRALPAK® and CHIRALCEL® available from DAICEL Corp. using the appropriate solvent or mixture of solvents to achieve good separation). If the compound contains a double bond, the substituent may be E or Z configuration. If the compound contains a disubstituted cycloalkyl, the cycloalkyl substituent may have a cis- or trans-configuration. All tautomeric forms are also intended to be included.

### Methods of Use

The compounds disclosed herein have EBP inhibitory activity. As used herein, “EBP inhibitory activity” refers to the ability of a compound or composition to induce a detectable decrease in EBP activity in vivo or in vitro (e.g., at least 10% decrease in EBP activity as measured by a given assay such as the bioassay described in the examples and known in the art).

In certain embodiments, the present disclosure provides a method of treating a disease or disorder responsive to inhibition of EBP activity (referred herein as “EBP mediated disease or disorder” or “disease or disorder mediated by EBP”) in a subject in need of the treatment.

The method comprises administering to the subject a compound described herein (e.g., a compound described in any one of the first to forty-sixth embodiments) or a pharmaceutically acceptable salt thereof or a pharmaceutical composition thereof.

In certain embodiments, the present disclosure provides the use of a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for the manufacture of a medicament for the treatment of a EBP mediated disorder or disease in a subject in need of the treatment.

In certain embodiments, the present disclosure provides a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for use in the treatment of a EBP mediated disorder or disease in a subject in need of the treatment.

In certain embodiments, the EBP mediated disorder is colorectal cancer.

In certain embodiments, the present disclosure provides a method of treating an autoimmune disease in a subject in need of the treatment. The method comprises administering to the subject a compound described herein (e.g., a compound described in any one of the first to forty-sixth embodiments) or a pharmaceutically acceptable salt thereof or a pharmaceutical composition thereof.

In certain embodiments, the present disclosure provides the use of a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for the manufacture of a medicament for the treatment of an autoimmune disease in a subject in need of the treatment.

In certain embodiments, the present disclosure provides a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for use in the treatment of an autoimmune disease in a subject in need of the treatment.

In certain embodiments, the autoimmune disease is multiple sclerosis (MS). The compounds of the present disclosure can be used for treating all stages of MS, including relapsing multiple sclerosis (or relapsing form(s) of multiple sclerosis), relapsing-remitting multiple sclerosis, primary progress multiple sclerosis, secondary progressive multiple sclerosis and clinically isolated syndrome (hereinafter “CIS”).

Relapsing multiple sclerosis (or relapsing form(s) of multiple sclerosis) includes clinically isolated syndrome, relapsing-remitting multiple sclerosis and active secondary progressive multiple sclerosis.

Relapsing-remitting multiple sclerosis is a stage of MS characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with multiple sclerosis.

Secondary progressive multiple sclerosis occurs in around 65% of those with initial relapsing-remitting multiple sclerosis, who eventually have progressive neurologic decline between acute attacks without any definite periods of remission. Occasional relapses and minor remissions may appear. The most common length of time between disease onset and conversion from relapsing-remitting to secondary progressive multiple sclerosis is 19 years.

Primary progressive multiple sclerosis is characterized by the same symptoms of secondary progressive multiple sclerosis, i.e., progressive neurologic decline between acute attacks without any definite periods of remission, without the prior relapsing-remitting stage.

CIS is a first episode of neurologic symptoms caused by inflammation and demyelination in the central nervous system. The episode, which by definition must last for at least 24 hours, is characteristic of multiple sclerosis but does not yet meet the criteria for a diagnosis of MS because people who experience a CIS may or may not go on to develop MS.

When CIS is accompanied by lesions on a brain MRI (magnetic resonance imaging) that are similar to those seen in MS, the person has a high likelihood of a second episode of neurologic symptoms and diagnosis of relapsing-remitting MS. When CIS is not accompanied by MS-like lesions on a brain MRI, the person has a much lower likelihood of developing MS.

In certain embodiments, the present disclosure provides a method of promoting myelination in a subject with a myelin-related disease or disorder in a subject in need of the treatment. The method comprises administering to the subject a compound described herein (e.g., a compound described in any one of the first to forty-sixth embodiments) or a pharmaceutically acceptable salt thereof or a pharmaceutical composition thereof.

In certain embodiments, the present disclosure provides the use of a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for the manufacture of a medicament for promoting myelination in a subject with a myelin-related disease or disorder in a subject in need of the treatment.

In certain embodiments, the present disclosure provides a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for use in promoting myelination in a subject with a myelin-related disease or disorder in a subject in need of the treatment.

In certain embodiments, the myelin-related disease or disorder is selected from multiple sclerosis (MS), neuromyelitis optica (NMO), optic neuritis, pediatric leukodystrophies, neonatal white matter injury, age-related dementia, schizophrenia, progressive multifocal leukoencephalopathy (PML), encephalomyelitis (EPL), acute disseminated encephalomyelitis (ADEM), central pontine myelolysis (CPM), adrenoleukodystrophy, Alexander's disease, Pelizaeus Merzbacher disease (PMD), Vanishing White Matter Disease, Wallerian Degeneration, transverse myelitis, amylotrophic lateral sclerosis (ALS), Huntington's disease, Alzheimer's disease, Parkinson's disease, spinal cord injury, traumatic brain injury, post radiation injury, neurologic complications of chemotherapy, stroke, acute ischemic optic neuropathy, vitamin E deficiency, isolated vitamin E deficiency syndrome, Bassen-Kornzweig syndrome, Marchiafava-Bignami syndrome, autism, metachromatic leukodystrophy, trigeminal neuralgia, acute disseminated encephalitis, chronic inflammatory demyelinating polyneuropathy, Guillian-Barre syndrome, Charcot-Marie-Tooth disease, Bell's palsy and radiation-induced demyelination, for example, neuromyelitis optica (NMO), optic neuritis, pediatric leukodystrophies, neonatal white matter injury, age-related dementia, and schizophrenia.

In certain embodiments, the present disclosure provides a method of treating cancer in a subject in need of the treatment. The method comprises administering to the subject a compound described herein (e.g., a compound described in any one of the first to forty-sixth embodiments) or a pharmaceutically acceptable salt thereof or a pharmaceutical composition thereof.

In certain embodiments, the present disclosure provides the use of a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for the manufacture of a medicament for the treatment of cancer in a subject in need of the treatment.

In certain embodiments, the present disclosure provides a compound described herein (e.g., a compound described in any one of the first to forty-sixth embodiments) or a pharmaceutically acceptable salt thereof or a pharmaceutical composition comprising a compound described herein or a pharmaceutically acceptable salt thereof for use in treating cancer in a subject in need of the treatment.

In certain embodiments, the cancer is colorectal cancer.

In certain embodiments, the present disclosure relates to the aforementioned methods, wherein said subject is a mammal. In certain embodiments, the subject is a primate. In certain embodiments, the subject is a human.

As used herein, an “effective amount” and a “therapeutically effective amount” can used interchangeably. It means an amount effective for treating or lessening the severity of one or more of the diseases, disorders or conditions as recited herein. In some embodiments, the effective dose can be between 10 μg and 500 mg.

The compounds and compositions, according to the methods of the present disclosure, may be administered using any amount and any route of administration effective for treating or lessening the severity of one or more of the diseases, disorders or conditions recited above.

In certain embodiments, the present disclosure relates to the aforementioned methods, wherein said compound is administered parenterally. In certain embodiments, the present disclosure relates to the aforementioned methods, wherein said compound is administered intramuscularly, intravenously, subcutaneously, orally, pulmonary, rectally, intrathecally, topically or intranasally. In certain embodiments, the present disclosure relates to the aforementioned methods, wherein said compound is administered systemically.

The compounds of the present invention can be used as a pharmaceutical composition (e.g., a compound of the present invention and at least one pharmaceutically acceptable carrier).

As used herein, the term “pharmaceutically acceptable carrier” includes generally recognized as safe (GRAS) solvents, dispersion media, surfactants, antioxidants, preservatives (e.g., antibacterial agents, antifungal agents), isotonic agents, salts, preservatives, drug stabilizers, buffering agents (e.g., maleic acid, tartaric acid, lactic acid, citric acid, acetic acid, sodium bicarbonate, sodium phosphate, and the like), and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated. For purposes of this disclosure, solvates and hydrates are considered pharmaceutical compositions comprising a compound of the present invention and a solvent (i.e., solvate) or water (i.e., hydrate).

The formulations may be prepared using conventional dissolution and mixing procedures. For example, the bulk drug substance (i.e., compound of the present invention or stabilized form of the compound (e.g., complex with a cyclodextrin derivative or other known complexation agent)) is dissolved in a suitable solvent in the presence of one or more of the excipients described above. The compound of the present invention is typically formulated into pharmaceutical dosage forms to provide an easily controllable dosage of the drug and to give the patient an elegant and easily handleable product.

The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical composition comprising a compound of the present disclosure is generally formulated for use as a parenteral or oral administration or alternatively suppositories.

For example, the pharmaceutical oral compositions of the present disclosure can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethylene glycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include a compound of the disclosure in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

The parenteral compositions (e.g, intravenous (IV) formulation) are aqueous isotonic solutions or suspensions. The parenteral compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are generally prepared according to conventional mixing, granulating or coating methods, respectively, and contain about 0.1-75%, or contain about 1-50%, of the active ingredient.

The compound of the present disclosure or pharmaceutical composition thereof for use in a subject (e.g., human) is typically administered orally or parenterally at a therapeutic dose.

When administered intravenously via infusion, the dosage may depend upon the infusion rate at which an IV formulation is administered. In general, the therapeutically effective dosage of a compound, the pharmaceutical composition, or the combinations thereof, is dependent on the species of the subject, the body weight, age and individual condition, the disorder or disease or the severity thereof being treated. A physician, pharmacist, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., aqueous solutions, and in vivo either enterally, parenterally, advantageously intravenously, e.g., as a suspension or in aqueous solution. The dosage in vitro may range between about 10-3 molar and 10-9 molar concentrations.

### Definitions

As used herein, a “patient,” “subject” or “individual” are used interchangeably and refer to either a human or non-human animal. The term includes mammals such as humans.

Typically, the animal is a mammal. A subject also refers to for example, primates (e.g., humans, male or female), cows, sheep, goats, horses, dogs, cats, rabbits, rats, mice, fish, birds and the like. In certain embodiments, the subject is a primate. Preferably, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease, condition or disorder, refers to the management and care of a patient for the purpose of combating the disease, condition, or disorder and includes the administration of a compound of the present invention to obtaining desired pharmacological and/or physiological effect. The effect can be therapeutic, which includes achieving, partially or substantially, one or more of the following results: partially or totally reducing the extent of the disease, condition or disorder; ameliorating or improving a clinical symptom, complications or indicator associated with the disease, condition or disorder; or delaying, inhibiting or decreasing the likelihood of the progression of the disease, condition or disorder; or eliminating the disease, condition or disorder. In certain embodiments, the effect can be to prevent the onset of the symptoms or complications of the disease, condition or disorder.

As used herein, the term “cancer” has the meaning normally accepted in the art. The term can broadly refer to abnormal cell growth.

As used herein, the term “autoimmune disease” has the meaning normally accepted the art. The term can broadly refer to a disease where the host's immune system targets or attacks normal or healthy tissue of the host.

As used herein, the term “myelination” has the meaning normally accepted in the art.

The term can broadly mean the process by which myelin is produced.

As used herein, the term “myelin-related disease or disorder”, “demyelinating disorder”, or “demyelation disorder” has the meaning normally accepted in the art. These terms can broadly refer to diseases or disorders which involve damage to myelin.

As used herein, a subject is “in need of” a treatment if such subject would benefit biologically, medically or in quality of life from such treatment (preferably, a human).

As used herein, the phrase “optionally substituted” is used interchangeably with the phrase “substituted or unsubstituted.” In general the term “optionally substituted” refers to the replacement of hydrogen radicals in a given structure with the radical of a specified substituent.

Specific substituents are described in the definitions and in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. In some embodiments, an optionally substituted group can be substituted with one or more substituents, each of which can the same or different. In some embodiments, the “one or more” substituents can be 1, 2, 3, 4, 5, 6, etc. substituents, each of which can the same or different. In some embodiment, the “one or more” substituents can be 1 to 6, 1 to 4, 1 to 3 or 1 to 2 substituents, each of which can the same or different.

As used herein, the term “alkyl” refers to a fully saturated branched or unbranched hydrocarbon moiety. The term “C₁₋₄alkyl” refers to an alkyl having 1 to 4 carbon atoms. The terms “C₁₋₃alkyl” and “C₁₋₂alkyl” are to be construed accordingly. Representative examples of “C₁₋₄alkyl” include, but are not limited to, methyl, ethyl, n-propyl, iso-propyl, n-butyl, sec-butyl, iso-butyl, and tert-butyl. Similarly, the alkyl portion (i.e., alkyl moiety) of an alkoxy have the same definition as above. When indicated as being “optionally substituted”, the alkane radical or alkyl moiety may be unsubstituted or substituted with one or more substituents (generally, one to three substituents except in the case of halogen substituents such as perchloro or perfluoroalkyls).

As used herein, the term “alkoxy” refers to a fully saturated branched or unbranched alkyl moiety attached through an oxygen bridge (i.e. a —O—C₁₋₄ alkyl group wherein C₁₋₄ alkyl is as defined herein). Representative examples of alkoxy include, but are not limited to, methoxy, ethoxy, propoxy, 2-propoxy, butoxy, tert-butoxy and the like. Preferably, alkoxy groups have about 1-4 carbons, more preferably about 1-2 carbons. The term “C₁₋₂ alkoxy” is to be construed accordingly.

As used herein, the term “C₁₋₄ alkoxyC₁₋₄ alkyl” refers to a C₁₋₄alkyl group as defined herein, wherein at least of the hydrogen atoms is replaced by an C₁₋₄alkoxy. The C₁₋₄alkoxyC₁₋₄ alkyl group is connected through the rest of the molecule described herein through the alkyl group.

The number of carbon atoms in a group is specified herein by the prefix “Cₓ₋ₓₓ”, wherein x and xx are integers. For example, “C₁₋₃ alkyl” is an alkyl group which has from 1 to 3 carbon atoms.

“Halogen” or “halo” may be fluorine, chlorine, bromine or iodine.

As used herein, the term “halo-substituted-C₁₋₄alkyl” or “C₁₋₄haloalkyl” refers to a C₁₋₄ alkyl group as defined herein, wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl.

Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “aryl” refers to an aromatic carbocyclic single ring or two fused ring system containing 6 to 10 carbon atoms. Examples include phenyl and naphthyl.

The term “heteroaryl” refers to a 5- to 12-membered aromatic radical containing 1-4 heteroatoms selected from N, O, and S. In some instances, nitrogen atoms in a heteroaryl may be quaternized. The term “heteroaryl” may be used interchangeably with the terms “heteroaryl ring”, “heteroaryl group”, or “heteroaromatic”. A heteroaryl group may be mono- or bi-cyclic.

Monocyclic heteroaryl includes, for example, pyrazolyl, imidazolyl, oxazolyl, pyridinyl, furanyl, oxadiazolyl, thiophenyl, and the like. Bi-cyclic heteroaryls include groups in which a monocyclic heteroaryl ring is fused to one or more aryl or heteroaryl rings. Non-limiting examples include pyrazolopyridinyl, pyrazolopyridinyl, benzotriazolyl, imidazopyridinyl, and indoyl.

The term “carbocyclic ring” or “carbocyclyl” refers to a 4- to 12-membered saturated or partially unsaturated hydrocarbon ring and may exist as a single ring, bicyclic ring (including fused, spiral or bridged carbocyclic rings) or a spiral ring. Bi-cyclic carbocyclyl groups include, e.g., unsaturated carbocyclic radicals fused to another unsaturated carbocyclic radical, cycloalkyl, or aryl, such as, for example, 2,3-dihydroindenyl, decahydronaphthalenyl, and 1,2,3,4-tetrahydronaphthalenyl. Unless specified otherwise, the carbocyclic ring generally contains 4- to 10-ring members.

The term “C₃₋₆ cycloalkyl” refers to a carbocyclic ring which is fully saturated (e.g., cyclopropyl, cyclobutyl, cyclopentyl, and cyclohexyl).

The term “heterocycle” or “heterocyclyl” refers to a 4- to 12-membered saturated or partially unsaturated heterocyclic ring containing 1 to 4 heteroatoms independently selected from N, O, and S. A heterocyclyl group may be mono- or bicyclic (e.g., a bridged, fused, or spiro bicyclic ring). Examples of monocyclic saturated or partially unsaturated heterocyclic radicals include, without limitation, oxetanyl, tetrahydrofuranyl, tetrahydropyranyl, and piperdinyl. Bi-cyclic heterocyclyl groups include, e.g., unsaturated heterocyclic radicals fused to another unsaturated heterocyclic radical, cycloalkyl, aryl, or heteroaryl ring, such as, for example, tetrahydro-3H-[1,2,3]triazolo[4,5-c]pyridinyl, 2-oxa-6-azaspiro[3.3]heptanyl, 5-oxabicyclo[2.1.1]hexanyl and 9-azabicyclo[3.3.1]nonanyl. In some embodiments, the heterocyclyl group is a 4 to 6 membered monocyclic heterocyclyl group. In some embodiments, the heterocyclyl group is a 4 to 6 membered monocyclic saturated heterocyclyl group. In some embodiments, the heterocyclyl group is a 8 to 10 membered bicyclic heterocyclyl group. In some embodiments, the heterocyclyl group is a 8 to 10 membered bicyclic saturated heterocyclyl group.

As used herein the term “spiral” ring means a two-ring system wherein both rings share one common atom. Examples of spiral rings include, 2-oxa-6-azaspiro[3.3]heptanyl and the like.

The term “fused” ring refers to two ring systems share two adjacent ring atoms. Fused heterocycles have at least one the ring systems contain a ring atom that is a heteroatom selected from O, N and S (e.g., 3-oxabicyclo[3.1.0]hexane).

As used herein the term “bridged” refers to a 5 to 10 membered cyclic moiety connected at two non-adjacent ring atoms (e.g. 5-oxabicyclo[2.1.1]hexane).

The phrase “pharmaceutically acceptable” indicates that the substance, composition or dosage form must be compatible chemically and/or toxicologically, with the other ingredients comprising a formulation, and/or the mammal being treated therewith.

Unless specified otherwise, the term “compounds of the present disclosure” refers to compounds of Formula (I), as well as all stereoisomers (including diastereoisomers and enantiomers), rotamers, tautomers, isotopically labeled compounds (including deuterium substitutions). When a moiety is present that is capable of forming a salt, then salts are included as well, in particular pharmaceutically acceptable salts.

As used herein, the term “a,” “an,” “the” and similar terms used in the context of the present invention (especially in the context of the claims) are to be construed to cover both the singular and plural unless otherwise indicated herein or clearly contradicted by the context.

The use of any and all examples, or exemplary language (e.g., “such as”) provided herein is intended merely to better illuminate the invention and does not pose a limitation on the scope of the invention otherwise claimed.

It is also possible that the intermediates and compounds of the present invention may exist in different tautomeric forms, and all such forms are embraced within the scope of the invention. The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. A specific example of a proton tautomer is the imidazole moiety where the proton may migrate between the two ring nitrogens. Valence tautomers include interconversions by reorganization of some of the bonding electrons.

In one embodiment, the present disclosure relates to a compound of the Formula (I) as defined herein, in free form. In another embodiment, the present disclosure relates to a compound of the Formula (I) as defined herein, in salt form. In another embodiment, the present disclosure relates to a compound of the Formula (I) as defined herein, in acid addition salt form.

In a further embodiment, the present disclosure relates to a compound of the Formula (I) as defined herein, in pharmaceutically acceptable salt form. In yet a further embodiment, the present disclosure relates to a compound of the Formula (I) as defined herein, in pharmaceutically acceptable acid addition salt form. In yet a further embodiment, the present disclosure relates to any one of the compounds of the Examples in free form. In yet a further embodiment, the present disclosure relates to any one of the compounds of the Examples in salt form. In yet a further embodiment, the present disclosure relates to any one of the compounds of the Examples in acid addition salt form. In yet a further embodiment, the present disclosure relates to any one of the compounds of the Examples in pharmaceutically acceptable salt form. In still another embodiment, the present disclosure relates to any one of the compounds of the Examples in pharmaceutically acceptable acid addition salt form.

Compounds of the present disclosure may be synthesized by synthetic routes that include processes analogous to those well-known in the chemical arts, particularly in light of the description contained herein. The starting materials are generally available from commercial sources such as Sigma-Aldrich or are readily prepared using methods well known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database)).

For illustrative purposes, the reaction schemes depicted below provide potential routes for synthesizing the compounds of the present disclosure as well as key intermediates. For a more detailed description of the individual reaction steps, see the Examples section below.

Although specific starting materials and reagents are depicted in the schemes and discussed below, other starting materials and reagents can be easily substituted to provide a variety of derivatives and/or reaction conditions.

### EXEMPLIFICATION

### Abbreviations

PE=petroleum ether
EtOAc=EA=ethyl acetate
ESI=electrospray ionisation
MeOH=methanol
EtOH=ethanol
DCE=1,2-dichloroethane
DCM=dichloromethane
CHCl₃=chloroform
HCl=hydrochloric acid
H₂O=water
IPA=isopropyl alcohol
LCMS=liquid chromatography mass spectrometry
HFIP=hexafluoro-2-propanol
HPLC=high pressure liquid chromatography
THF=tetrahydrofuran
MeCN=ACN=acetonitrile
MgSO₄=magnesium sultate
DMSO=dimetylsulfoxide
AcOH=acetic acid
TFA=trifluoroacetic acid
DIPEA=diisopropylethyl amine
N₂=Nitrogen
NH₄HCO₃=Ammonium Bicarbonate
t-BuOH=tert-butanol
NH₄Cl=ammonium chloride
NaH=sodium hydride
Na₂SO₄=sodium sulfate
K₂CO₃=potassium carbonate
NaHCO₃=sodium bicarbonate
NaBH(OAc)₃=STAB=sodium triacetoxyborohydride
SiO₂=silicon dioxide or silica
PDA=Photo Diode Array Detection
TosMIC=toluenesulfonylmethyl isocyanide
TLC=Thin Layer Chromatography
LiHMDS=Lithium bis(trimethylsilyl)amide

### General Methods

### QC Analysis LC/MS Method Conditions:

### Ammonium Hydroxide (Basic pH) Conditions

MS mode: MS:ESI+scan range 165-650 daltons
PDA: 200-400 nm scan range
Column: Waters ACQUITY UPLC BEH C18 2.1×50 mm, 1.7 μm; Part No. 186002350
Modifier: Ammonium hydroxide 0.2% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### Trifluoroacetic Acid (Acidic pH) Conditions

MS mode: MS:ESI+scan range 165-650 daltons
PDA: 200-400 nm scan range
Column: Waters ACQUITY UPLC BEH C18 2.1×50 mm, 1.7 μm; Part No. 186002350
Modifier: Trifluoroacetic acid 0.1% (v/v) conc.
Method: 95% water/5% MeCN (initial conditions) linear gradient to 5% water/95% MeCN at 3.75 min, HOLD 5% water/95% MeCN to 4 min. Flow rate, 0.8 mL/min.

### General Prep HPLC Conditions:

### Ammonium Hydroxide (Basic pH) Conditions

Flow rate: 30 mL/min
MS mode: MS:ESI+scan range 165-650 daltons
PDA: 200-400 nm scan range
Column: Waters XSELECT CSH C18 PREP 19×100 mm, 5 μm; Part No. 186005421
Modifier: 0.2% Ammonium hydroxide (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate: 50 mL/min
MS mode: MS:ESI+scan range 165-650 daltons
PDA: 200-400 nm scan range
Column: Waters XSELECT CSH C18 PREP 30×100 mm, 5 μm; Part No. 186005425
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+scan range 165-650 daltons
PDA: 200-400 nm scan range
Column: Waters XSELECT CSH C18 PREP 30×50 mm, 5 μm; Part No. 186005423
Modifier: 0.2% NH₄OH (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### Trifluoroacetic Acid (Acidic pH) Conditions

Flow rate, 30 mL/min
MS mode: MS:ESI+scan range 165-650 daltons
PDA: 200-400 nm scan range
Column: Waters Sunfire OBD C18 PREP 19×100 mm, 5 μm; Part No. 186002567
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 50 mL/min
MS mode: MS:ESI+scan range 165-650 daltons
PDA: 200-400 nm scan range
Column: Waters Sunfire OBD C18 PREP 30×100 mm, 5 μm; Part No. 186002572
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.
Flow rate, 60 mL/min
MS mode: MS:ESI+scan range 165-650 daltons
PDA: 200-400 nm scan range
Column: Waters Sunfire OBD C18 PREP 30×50 mm, 5 μm; Part No. 186002570
Modifier: 0.1% Trifluoroacetic acid (v/v) conc.
Method: A % water/B % MeCN (initial conditions) linear gradient to A % water/B % MeCN at 8 min, ramp to 5% water/95% MeCN at 8.5 min, HOLD 5% water/95% MeCN to 10 min.

### EXPERIMENTAL PROCEDURES

### Example 1: (3aR,5r,6aS)-2-((2,4-Dimethylphenyl)sulfonyl)-N-((tetrahydro-2H-pyran-4-yl)methyl)octahydrocyclopenta[c]pyrrol-5-amine

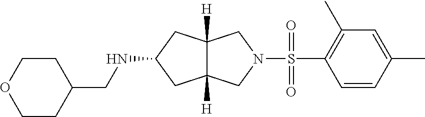 [SMILES: Cc1ccc(S(=O)(=O)N2C[C@H]3C[C@@H](NCC4CCOCC4)C[C@H]3C2)c(C)c1]

### 1. Synthesis of tert-butyl (3aR,5r,6aS)-5-(((tetrahydro-2H-pyran-4-yl)methyl)amino)hexahydrocyclopenta[c]pyrrole-2(1H)-carboxylate

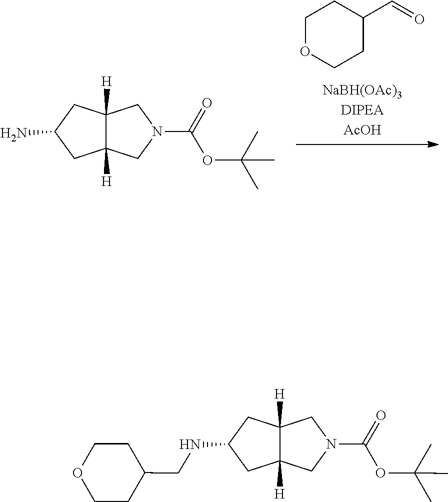 [SMILES: O=CC1CCOCC1 ; CC(C)(C)OC(=O)N1C[C@H]2C[C@@H](N)C[C@H]2C1 ; CC(C)(C)OC(=O)N1C[C@H]2C[C@@H](NCC3CCOCC3)C[C@H]2C1]

To a mixture of tert-butyl (3aR,5r,6aS)-5-aminohexahydrocyclopenta[c]pyrrole-2(1H)-carboxylate (213 mg, 0.9 mmol) and tetrahydro-2H-pyran-4-carbaldehyde (102 mg, 0.9 mmol) in DCM (5 mL) was added DIPEA (424 μL, 2.4 mmol). After stirring for 15 min, acetic acid (139 μL, 2.4 mmol) was added. After stirring for 15 min, sodium triacetoxyborohydride (687 mg, 3.2 mmol) was added and the reaction was stirred at rt for 1 day. Saturated NaHCO₃ solution and saturated sodium chloride solution were carefully added and the mixture was extracted with EtOAc. Solvents were evaporated to dryness and the residue was purified by SCX (eluting with 2N NH₃ in MeOH) to give tert-butyl (3aR,5r,6aS)-5-(((tetrahydro-2H-pyran-4-yl)methyl)amino)hexahydrocyclopenta[c]pyrrole-2(1H)-carboxylate (239 mg, 78%) that was not purified further. LCMS m/z=325.2 [M+H]⁺.

### 2. Synthesis of tert-butyl (3aR,5r,6aS)-5-(((benzyloxy)carbonyl)((tetrahydro-2H-pyran-4-yl)methyl)amino)hexahydrocyclopenta[c]pyrrole-2(1H)-carboxylate

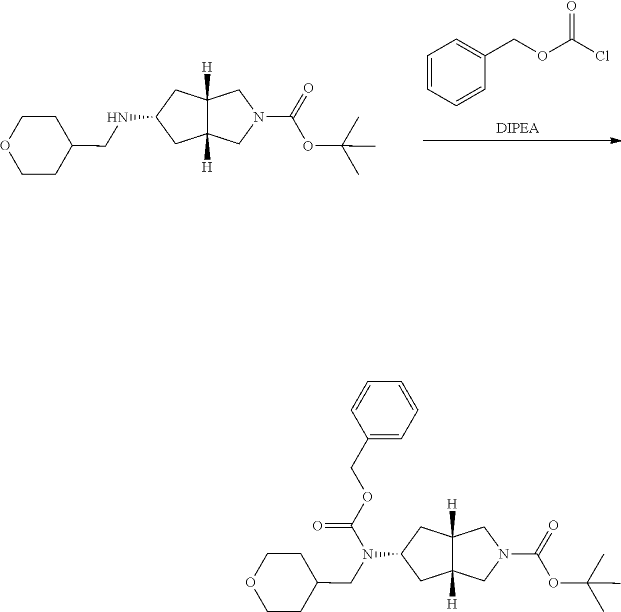 [SMILES: CC(C)(C)OC(=O)N1C[C@H]2C[C@@H](NCC3CCOCC3)C[C@H]2C1 ; O=C(Cl)OCc1ccccc1 ; CC(C)(C)OC(=O)N1C[C@H]2C[C@@H](N(CC3CCOCC3)C(=O)OCc3ccccc3)C[C@H]2C1]

To a solution of tert-butyl (3aR,5r,6aS)-5-(((tetrahydro-2H-pyran-4-yl)methyl)amino)hexahydrocyclopenta[c]pyrrole-2(1H)-carboxylate (120 mg, 0.4 mmol) in DCM (2.5 mL) was added DIPEA (225 μL, 1.3 mmol) followed by benzyl chloroformate (184 μL, 0.6 mmol) as 3 M solution in toluene. After stirring at rt for 4 h, the reaction was quenched by careful addition of saturated NaHCO₃ solution. The mixture was extracted with DCM and the organics were evaporated. The residue was purified by silica gel chromatography (0-100% EtOAc in heptanes) to give tert-butyl (3aR,5r,6aS)-5-(((benzyloxy)carbonyl)((tetrahydro-2H-pyran-4-yl)methyl)amino)hexahydrocyclopenta[c]pyrrole-2(1H)-carboxylate (119 mg, 70%).

LCMS m/z=481.3 [M+Na]⁺.

### 3. Synthesis of benzyl ((3aR,5r,6aS)-octahydrocyclopenta[c]pyrrol-5-yl)((tetrahydro-2H-pyran-4-yl)methyl)carbamate

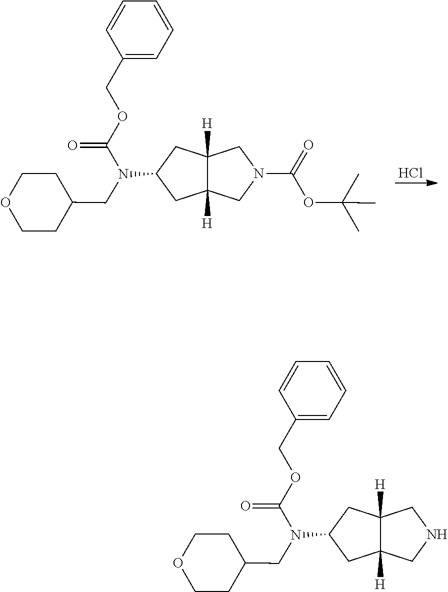 [SMILES: CC(C)(C)OC(=O)N1C[C@H]2C[C@@H](N(CC3CCOCC3)C(=O)OCc3ccccc3)C[C@H]2C1 ; Cl ; O=C(OCc1ccccc1)N(CC1CCOCC1)[C@H]1C[C@H]2CNC[C@H]2C1]

To a solution of tert-butyl (3aR,5r,6aS)-5-(((benzyloxy)carbonyl)((tetrahydro-2H-pyran-4-yl)methyl)amino)hexahydrocyclopenta[c]pyrrole-2(1H)-carboxylate (119 mg, 0.3 mmol) in EtOAc (2 mL) was added HCl (298 μL, 1.2 mmol) as 4 M dioxane solution. After stirring at rt for 1 day, the reaction was evaporated to dryness to give benzyl ((3aR,5r,6aS)-octahydrocyclopenta[c]pyrrol-5-yl)((tetrahydro-2H-pyran-4-yl)methyl)carbamate (122 mg, 119%) as HCl salt that was used without further purification. LCMS m/z=359.2 [M+H]⁺.

### 4. Synthesis of benzyl ((3aR,5r,6aS)-2-((2,4-dimethylphenyl)sulfonyl)octahydrocyclopenta[c]pyrrol-5-yl)((tetrahydro-2H-pyran-4-yl)methyl)carbamate

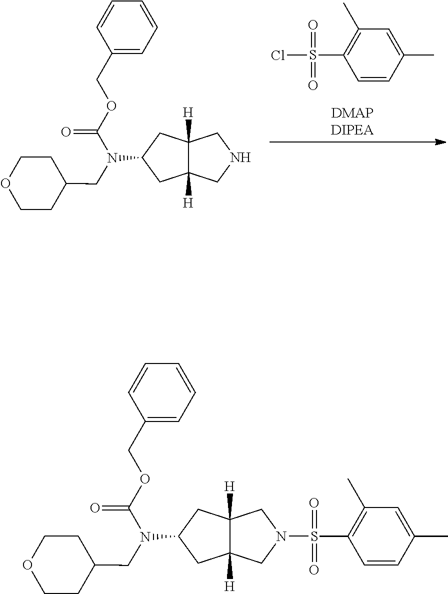 [SMILES: O=C(OCc1ccccc1)N(CC1CCOCC1)[C@H]1C[C@H]2CNC[C@H]2C1 ; Cc1ccc(S(=O)(=O)Cl)c(C)c1 ; Cc1ccc(S(=O)(=O)N2C[C@H]3C[C@@H](N(CC4CCOCC4)C(=O)OCc4ccccc4)C[C@H]3C2)c(C)c1]

To a mixture of benzyl ((3aR,5r,6aS)-octahydrocyclopenta[c]pyrrol-5-yl)((tetrahydro-2H-pyran-4-yl)methyl)carbamate (51 mg, 0.1 mmol) as HCl salt in THE (1.5 mL) was added DIPEA (113 μL, 0.6 mmol) followed by DMAP (16 mg, 0.1 mmol) and then 2,4-dimethylbenzenesulfonyl chloride (40 mg, 0.2 mmol). After stirring at rt for 2.5 h, the reaction was quenched by careful addition of saturated ammonium chloride solution. The mixture was extracted with EtOAc and the solvents were evaporated. The residue was purified by silica gel chromatography (0-100% EtOAc in heptanes) to give benzyl ((3aR,5r,6aS)-2-((2,4-dimethylphenyl)sulfonyl)octahydrocyclopenta[c]pyrrol-5-yl)((tetrahydro-2H-pyran-4-yl)methyl)carbamate (49 mg, 71%). LCMS m/z=527.2 [M+H]⁺.

### 5. Synthesis of (3aR,5r,6aS)-2-((2,4-dimethylphenyl)sulfonyl)-N-((tetrahydro-2H-pyran-4-yl)methyl)octahydrocyclopenta[c]pyrrol-5-amine

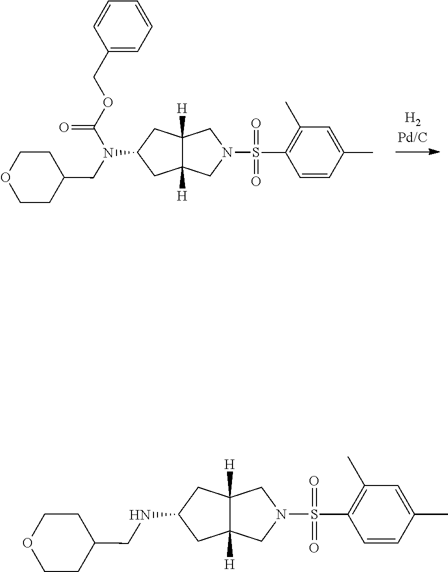 [SMILES: Cc1ccc(S(=O)(=O)N2C[C@H]3C[C@@H](N(CC4CCOCC4)C(=O)OCc4ccccc4)C[C@H]3C2)c(C)c1 ; Cc1ccc(S(=O)(=O)N2C[C@H]3C[C@@H](NCC4CCOCC4)C[C@H]3C2)c(C)c1]

To a solution of benzyl ((3aR,5r,6aS)-2-((2,4-dimethylphenyl)sulfonyl)octahydrocyclopenta[c]pyrrol-5-yl)((tetrahydro-2H-pyran-4-yl)methyl)carbamate (49 mg, 0.1 mmol) in MeOH (5 mL) was added 10% palladium on carbon (10 mg, 0.01 mmol). The reaction vessel was evacuated and refilled with H₂ (three times), then hydrogenated at balloon pressure of H₂ for 1 day. The reaction was filtered through Celite® and evaporated. The residue was purified by prep-HPLC (Waters Sunfire Prep C18 5 μm OBD 30×100 mm; Method: (A) 95% {H₂O}//(B) 5% {MeCN}w/0.1% TFA (initial conditions hold for 0.5 min) then a linear gradient to 40% (A)/60% (B) over 7.5 min (flow rate: 50 mL/min) to give (3aR,5r,6aS)-2-((2,4-dimethylphenyl)sulfonyl)-N-((tetrahydro-2H-pyran-4-yl)methyl)octahydrocyclopenta[c]pyrrol-5-amine (20 mg, 43%) as white solid (TFA salt).

LCMS m/z=393.3 [M+H]⁺. t_{R}=1.45 min (TFA). t_{R}=2.04 min (NH₄OH).

### Example 2: (3aR,5r,6aS)-2-((4-(Difluoromethoxy)phenyl)sulfonyl)-N-((tetrahydro-2H-pyran-4-yl)methyl)octahydrocyclopenta[c]pyrrol-5-amine

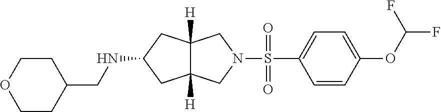 [SMILES: O=S(=O)(c1ccc(OC(F)F)cc1)N1C[C@H]2C[C@@H](NCC3CCOCC3)C[C@H]2C1]

### 1. Synthesis of benzyl ((3aR,5r,6aS)-2-((4-(difluoromethoxy)phenyl)sulfonyl)octahydrocyclopenta[c]pyrrol-5-yl)((tetrahydro-2H-pyran-4-yl)methyl)carbamate

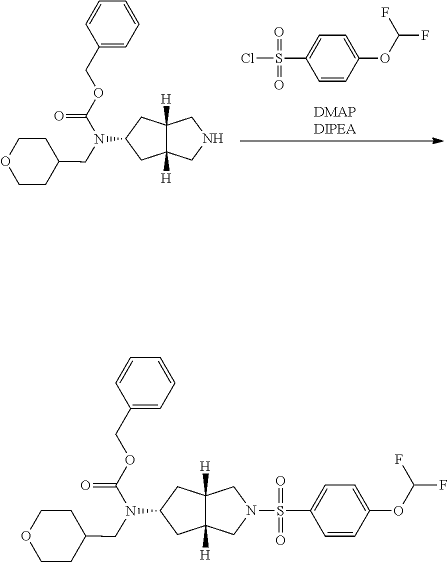 [SMILES: O=C(OCc1ccccc1)N(CC1CCOCC1)[C@H]1C[C@H]2CNC[C@H]2C1 ; O=S(=O)(Cl)c1ccc(OC(F)F)cc1 ; O=C(OCc1ccccc1)N(CC1CCOCC1)[C@@H]1C[C@@H]2CN(S(=O)(=O)c3ccc(OC(F)F)cc3)C[C@@H]2C1]

To a mixture of benzyl ((3aR,5r,6aS)-octahydrocyclopenta[c]pyrrol-5-yl)((tetrahydro-2H-pyran-4-yl)methyl)carbamate (51 mg, 0.1 mmol) (Example 1, step 3) as HCl salt in THF (1.5 mL) was added DIPEA (113 μL, 0.6 mmol) followed by DMAP (16 mg, 0.1 mmol) and then 4-(difluoromethoxy)benzenesulfonyl chloride (31 μL, 0.2 mmol). After stirring at rt for 3.5 h, the reaction was quenched by careful addition of saturated ammonium chloride solution. The mixture was extracted with EtOAc and the solvents were evaporated. The residue was purified by silica gel chromatography (0-100% EtOAc in heptanes) to give benzyl ((3aR,5r,6aS)-2-((4-(difluoromethoxy)phenyl)sulfonyl)octahydrocyclopenta[c]pyrrol-5-yl)((tetrahydro-2H-pyran-4-yl)methyl)carbamate (50 mg, 69%). LCMS m/z=565.3 [M+H]⁺.

### 2. Synthesis of (3aR,5r,6aS)-2-((4-(difluoromethoxy)phenyl)sulfonyl)-N-((tetrahydro-2H-pyran-4-yl)methyl)octahydrocyclopenta[c]pyrrol-5-amine

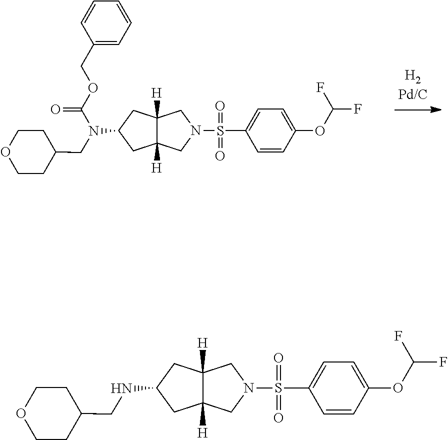 [SMILES: O=C(OCc1ccccc1)N(CC1CCOCC1)[C@@H]1C[C@@H]2CN(S(=O)(=O)c3ccc(OC(F)F)cc3)C[C@@H]2C1 ; O=S(=O)(c1ccc(OC(F)F)cc1)N1C[C@H]2C[C@@H](NCC3CCOCC3)C[C@H]2C1]

(3aR,5r,6aS)-2-((4-(Difluoromethoxy)phenyl)sulfonyl)-N-((tetrahydro-2H-pyran-4-yl)methyl)octahydrocyclopenta[c]pyrrol-5-amine was obtained as a clear oil, as a TFA salt (33 mg, 69%) from benzyl ((3aR,5r,6aS)-2-((4-(difluoromethoxy)phenyl)sulfonyl)octahydrocyclopenta[c]pyrrol-5-yl)((tetrahydro-2H-pyran-4-yl)methyl)carbamate, following the method described in Example 1, step 5. LCMS m/z=431.2 [M+H]⁺. t_{R}=1.38 min (TFA). t_{R}=1.88 min (NH₄OH).

### Example 3: (3aR,5s,6aS)-2-((2,4-Dimethylphenyl)sulfonyl)-5-(4-methylpiperidin-1-yl)octahydrocyclopenta[c]pyrrole

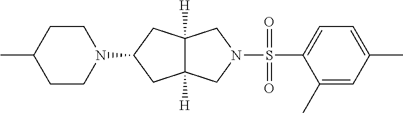

### 1. Synthesis of tert-butyl (3aR,5s,6aS)-5-(4-methylpiperidin-1-yl)hexahydrocyclopenta[c]pyrrole-2(1H)-carboxylate

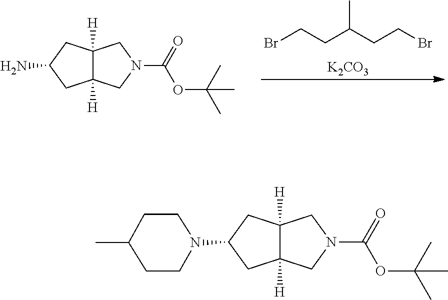

To a mixture of tert-butyl (3aR,5s,6aS)-5-aminohexahydrocyclopenta[c]pyrrole-2(1H)-carboxylate (350 mg, 1.3 mmol) as HCl salt and 1,5-dibromo-3-methylpentane (502 mg, 2.1 mmol) in MeCN (6 mL) was added K₂CO₃ (638 mg, 4.6 mmol). The reaction was heated at 90° C. for 19 h, then the reaction was cooled and filtered. The mixture was evaporated to dryness and purified by SCX (eluting with 2N NH₃ in MeOH) to give tert-butyl (3aR,5s,6aS)-5-(4-methylpiperidin-1-yl)hexahydrocyclopenta[c]pyrrole-2(1H)-carboxylate (352 mg, 86%).

LCMS m/z=309.1 [M+H]⁺.

### 2. Synthesis of (3aR,5s,6aS)-5-(4-methylpiperidin-1-yl)octahydrocyclopenta[c]pyrrole

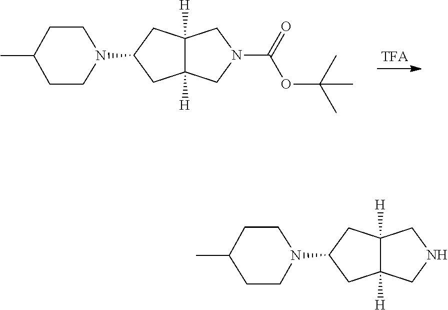

To a solution of tert-butyl (3aR,5s,6aS)-5-(4-methylpiperidin-1-yl)hexahydrocyclopenta[c]pyrrole-2(1H)-carboxylate (117 mg, 0.4 mmol) in DCM (2 mL) was added TFA (2 mL, 26 mmol). The reaction was stirred for 2 h at rt, then was evaporated to dryness. The residue was purified by SCX (eluting with 2N NH₃ in MeOH) to give (3aR,5s,6aS)-5-(4-methylpiperidin-1-yl)octahydrocyclopenta[c]pyrrole (75 mg, 95%). LCMS

m/z=209.2 [M+H]⁺.

### 3. Synthesis of (3aR,5s,6aS)-2-((2,4-dimethylphenyl)sulfonyl)-5-(4-methylpiperidin-1-yl)octahydrocyclopenta[c]pyrrole

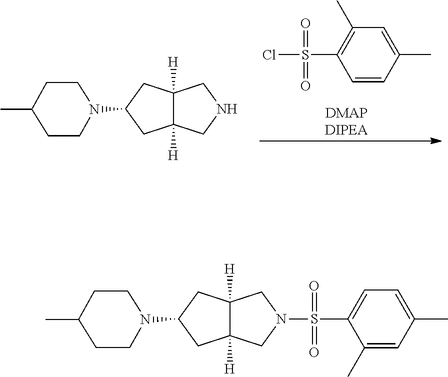

To a solution of (3aR,5s,6aS)-5-(4-methylpiperidin-1-yl)octahydrocyclopenta[c]pyrrole (38 mg, 0.2 mmol) in THE (2 mL) was added DIPEA (160 μL, 0.9 mmol) and DMAP (2 mg, 0.02 mmol), followed by 2,4-dimethylbenzenesulfonyl chloride (56 mg, 0.3 mmol). After 1 day stirring at rt, the reaction was quenched by slow addition of saturated NaHCO₃ solution. The mixture was extracted with EtOAc and the solvents were evaporated. The residue was purified by prep-HPLC (Waters Sunfire Prep C18 5 μm OBD 30×100 mm; Method: (A) 95% {H₂O}//(B) 5% {MeCN}w/0.1% TFA (initial conditions hold for 0.5 min) then a linear gradient to 35% (A)/65% (B) over 7.5 min (flow rate: 50 mL/min) to give (3aR,5s,6aS)-2-((2,4-dimethylphenyl)sulfonyl)-5-(4-methylpiperidin-1-yl)octahydrocyclopenta[c]pyrrole (59 mg, 66%) as a clear oil (TFA salt). LCMS m/z=377.2 [M+H]⁺. t_{R}=1.59 min (TFA). t_{R}=2.50 min (NH₄OH).

### Example 4: (3aR,5s,6aS)-2-((4-(Difluoromethoxy)phenyl)sulfonyl)-5-(4-methylpiperidin-1-yl)octahydrocyclopenta[c]pyrrole

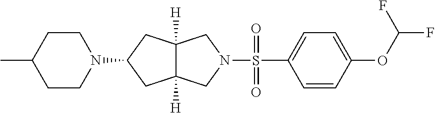 [SMILES: CC1CCN([C@H]2C[C@@H]3CN(S(=O)(=O)c4ccc(OC(F)F)cc4)C[C@@H]3C2)CC1]

### 1. Synthesis of (3aR,5s,6aS)-2-((4-(difluoromethoxy)phenyl)sulfonyl)-5-(4-methylpiperidin-1-yl)octahydrocyclopenta[c]pyrrole

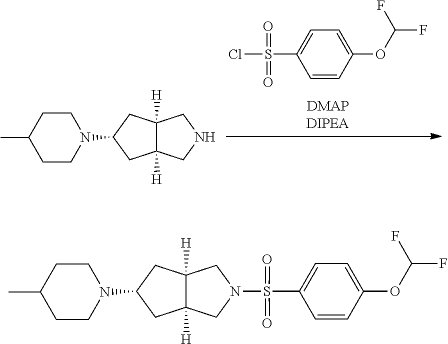 [SMILES: O=S(=O)(Cl)c1ccc(OC(F)F)cc1 ; CC1CCN([C@@H]2C[C@H]3CNC[C@H]3C2)CC1 ; CC1CCN([C@H]2C[C@@H]3CN(S(=O)(=O)c4ccc(OC(F)F)cc4)C[C@@H]3C2)CC1]

(3aR,5s,6aS)-2-((4-(Difluoromethoxy)phenyl)sulfonyl)-5-(4-methylpiperidin-1-yl)octahydrocyclopenta[c]pyrrole was obtained as a TFA salt (47 mg, 49%) from (3aR,5s,6aS)-5-(4-methylpiperidin-1-yl)octahydrocyclopenta[c]pyrrole (Example 3, step 2) and 4-(difluoromethoxy)benzenesulfonyl chloride, following the method described in Example 3, step 3. LCMS m/z=415.2 [M+H]⁺. t_{R}=1.52 min (TFA). t_{R}=2.27 min (NH₄OH).

### Example 5: (3aR,5r,6aS)-2-((2,4-Dimethylphenyl)sulfonyl)-5-(4-methylpiperidin-1-yl)octahydrocyclopenta[c]pyrrole

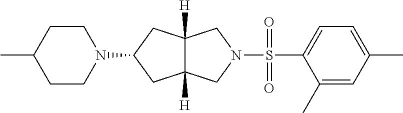

### 1. Synthesis of tert-butyl (3aR,5r,6aS)-5-(4-methylpiperidin-1-yl)hexahydrocyclopenta[c]pyrrole-2(1H)-carboxylate

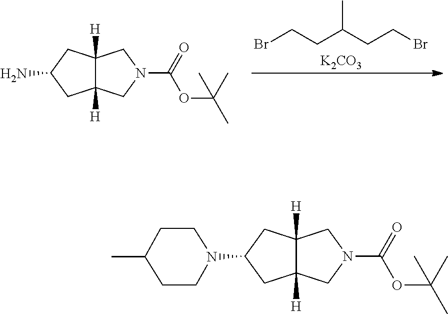

To a mixture of tert-butyl (3aR,5r,6aS)-5-aminohexahydrocyclopenta[c]pyrrole-2(11)-carboxylate (290 mg, 1.3 mmol) and 1,5-dibromo-3-methylpentane (483 mg, 2.0 mmol) in MeCN (6 mL) was added K₂CO₃ (614 mg, 4.4 mmol). The reaction was heated at 90° C. for 19 h, then the reaction was cooled and filtered. The mixture was evaporated to dryness and purified by SCX (eluting with 2N NH₃ in MeOH) to give tert-butyl (3aR,5r,6aS)-5-(4-methylpiperidin-1-yl)hexahydrocyclopenta[c]pyrrole-2(1H)-carboxylate (305 mg, 77%). LCMS m/z=309.1 [M+H]⁺.

### 2. Synthesis of (3aR,5r,6aS)-5-(4-methylpiperidin-1-yl)octahydrocyclopenta[c]pyrrole

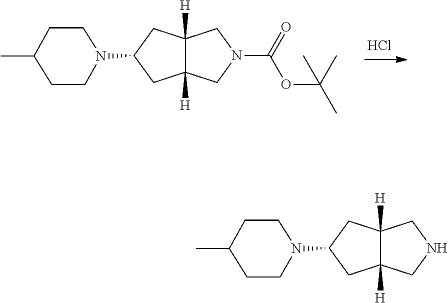

To a solution of tert-butyl (3aR,5r,6aS)-5-(4-methylpiperidin-1-yl)hexahydrocyclopenta[c]pyrrole-2(1H)-carboxylate (152 mg, 0.5 mmol) in EtOAc (3 mL) was added HCl (568 μL, 2.3 mmol) as 4 M solution in dioxane. The reaction was stirred for 5 h at rt, with frequent sonicating to reduce clumping. Additional HCl (568 μL, 2.3 mmol) as 4 M solution in dioxane was added and the reaction was stirred for an additional 1 day. The reaction was evaporated to dryness to give crude (3aR,5r,6aS)-5-(4-methylpiperidin-1-yl)octahydrocyclopenta[c]pyrrole (154 mg, 111%) as presumed bis-HCl salt. LCMS m/z=209.2 [M+H]⁺.

### 3. Synthesis of (3aR,5r,6aS)-2-((2,4-dimethylphenyl)sulfonyl)-5-(4-methylpiperidin-1-yl)octahydrocyclopenta[c]pyrrole

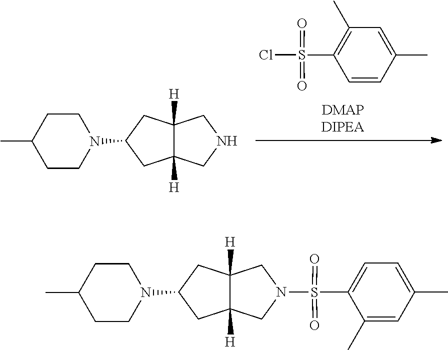

To a mixture of (3aR,5r,6aS)-5-(4-methylpiperidin-1-yl)octahydrocyclopenta[c]pyrrole (69 mg, 0.2 mmol) as bis-HCl salt in THE (2 mL) was added DIPEA (258 μL, 1.5 mmol) and DMAP (3 mg, 0.02 mmol), followed by 2,4-dimethylbenzenesulfonyl chloride (76 mg, 0.4 mmol). Additional DIPEA (258 μL, 1.5 mmol) was added to improve stirring. After 2.5 h stirring at rt, the reaction was quenched by slow addition of saturated NaHCO₃ solution. The mixture was extracted with EtOAc and the solvents were evaporated. The residue was purified by prep-HPLC (Waters Sunfire Prep C18 5 μm OBD 30×100 mm; Method: (A) 95% {H₂O}//(B) 5% {MeCN}w/0.1% TFA (initial conditions hold for 0.5 min) then a linear gradient to 40% (A)/60% (B) over 7.5 min (flow rate: 50 mL/min) to give (3aR,5r,6aS)-2-((2,4-dimethylphenyl)sulfonyl)-5-(4-methylpiperidin-1-yl)octahydrocyclopenta[c]pyrrole (47 mg, 39%) as a brown oil (TFA salt). LCMS m/z=377.4 [M+H]⁺. t_{R}=1.67 min (TFA). t_{R}=2.81 min (NH₄OH).

### Example 6: (3aR,5r,6aS)-2-((4-(Difluoromethoxy)phenyl)sulfonyl)-5-(4-methylpiperidin-1-yl)octahydrocyclopenta[c]pyrrole

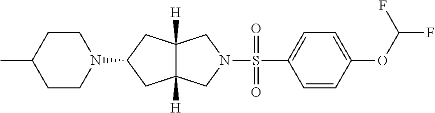 [SMILES: CC1CCN([C@@H]2C[C@@H]3CN(S(=O)(=O)c4ccc(OC(F)F)cc4)C[C@@H]3C2)CC1]

### 1. Synthesis of (3aR,5r,6aS)-2-((4-(difluoromethoxy)phenyl)sulfonyl)-5-(4-methylpiperidin-1-yl)octahydrocyclopenta[c]pyrrole

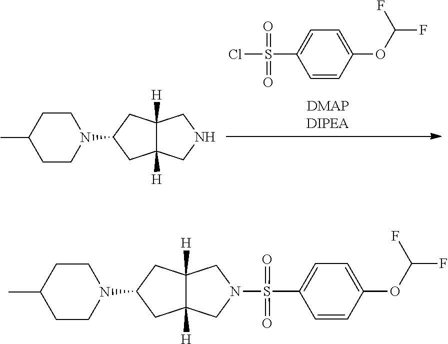 [SMILES: O=S(=O)(Cl)c1ccc(OC(F)F)cc1 ; CC1CCN([C@H]2C[C@H]3CNC[C@H]3C2)CC1 ; CC1CCN([C@@H]2C[C@@H]3CN(S(=O)(=O)c4ccc(OC(F)F)cc4)C[C@@H]3C2)CC1]

(3aR,5r,6aS)-2-((4-(Difluoromethoxy)phenyl)sulfonyl)-5-(4-methylpiperidin-1-yl)octahydrocyclopenta[c]pyrrole was obtained as a TFA salt, as a white solid (71 mg, 54%) from (3aR,5r,6aS)-5-(4-methylpiperidin-1-yl)octahydrocyclopenta[c]pyrrole bis-HCl salt (Example 5, step 2) and 4-(difluoromethoxy)benzenesulfonyl chloride, following a similar procedure to that described in Example 5, step 3, except the HPLC gradient used was from 95% {H₂O}/5% {MeCN}w/0.1% TFA to 45% {H₂O}/55% {MeCN}w/0.1% TFA. LCMS m/z=415.3 [M+H]⁺. t_{R}=1.63 min (TFA). t_{R}=2.58 min (NH₄OH).

### Example 7: (3aR,5s,6aS)-2-((2,4-Dimethylphenyl)sulfonyl)-N-((tetrahydro-2H-pyran-4-yl)methyl)octahydrocyclopenta[c]pyrrol-5-amine

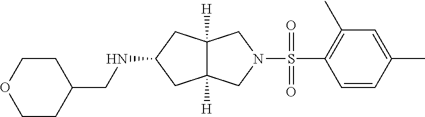 [SMILES: Cc1ccc(S(=O)(=O)N2C[C@H]3C[C@H](NCC4CCOCC4)C[C@H]3C2)c(C)c1]

### 1. Synthesis of tert-butyl (3aR,5s,6aS)-5-(((tetrahydro-2H-pyran-4-yl)methyl)amino)hexahydrocyclopenta[c]pyrrole-2(1H)-carboxylate

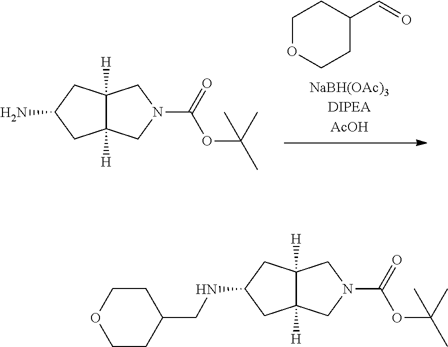 [SMILES: CC(C)(C)OC(=O)N1C[C@H]2C[C@H](N)C[C@H]2C1 ; O=CC1CCOCC1 ; CC(C)(C)OC(=O)N1C[C@H]2C[C@H](NCC3CCOCC3)C[C@H]2C1]

To a mixture of tert-butyl (3aR,5s,6aS)-5-aminohexahydrocyclopenta[c]pyrrole-2(1H)-carboxylate (300 mg, 1.1 mmol) as HCl salt and tetrahydro-2H-pyran-4-carbaldehyde (143 mg, 1.3 mmol) in DCM (6 mL) was added DIPEA (514 μL, 2.9 mmol). After stirring for 15 min, acetic acid (169 μL, 2.9 mmol) was added. After stirring for 15 min, sodium triacetoxyborohydride (968 mg, 4.6 mmol) was added and the reaction was stirred at rt for 1 day. Saturated NaHCO₃ solution and saturated sodium chloride solution were carefully added and the mixture was extracted with EtOAc. Solvents were evaporated to dryness and the residue was purified by SCX (eluting with 2N NH₃ in MeOH) to give tert-butyl (3aR,5s,6aS)-5-(((tetrahydro-2H-pyran-4-yl)methyl)amino)hexahydrocyclopenta[c]pyrrole-2(1H)-carboxylate (359 mg, 97%) that was not purified further. LCMS m/z=325.2 [M+H]⁺.

### 2. Synthesis of tert-butyl (3aR,5s,6aS)-5-(((benzyloxy)carbonyl)((tetrahydro-2H-pyran-4-yl)methyl)amino)hexahydrocyclopenta[c]pyrrole-2(H)-carboxylate

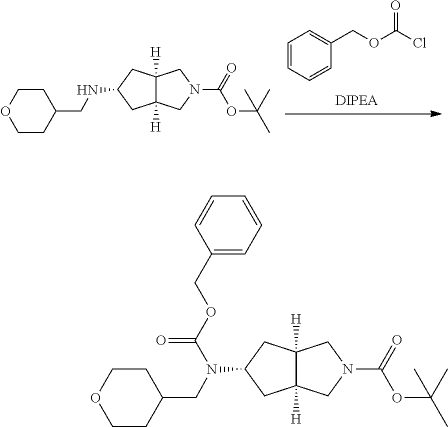

tert-Butyl (3aR,5s,6aS)-5-(((benzyloxy)carbonyl)((tetrahydro-2H-pyran-4-yl)methyl)amino)hexahydrocyclopenta[c]pyrrole-2(1H)-carboxylate was obtained (212 mg, 83%) from tert-butyl (3aR,5s,6aS)-5-(((tetrahydro-2H-pyran-4-yl)methyl)amino)hexahydrocyclopenta[c]pyrrole-2(1H)-carboxylate and benzyl chloroformate following the procedure described in Example 1, step 2. LCMS m/z=481.3 [M+Na]⁺.

### 3. Synthesis of benzyl ((3aR,5s,6aS)-octahydrocyclopenta[c]pyrrol-5-yl)((tetrahydro-2H-pyran-4-yl)methyl)carbamate

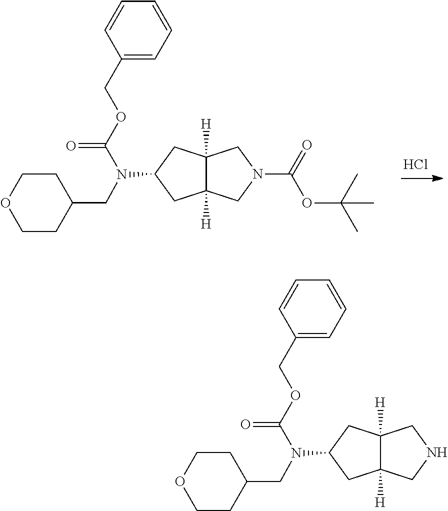 [SMILES: CC(C)(C)OC(=O)N1C[C@H]2C[C@H](N(CC3CCOCC3)C(=O)OCc3ccccc3)C[C@H]2C1 ; Cl ; O=C(OCc1ccccc1)N(CC1CCOCC1)[C@@H]1C[C@H]2CNC[C@H]2C1]

Benzyl ((3aR,5s,6aS)-octahydrocyclopenta[c]pyrrol-5-yl)((tetrahydro-2H-pyran-4-yl)methyl)carbamate was obtained as an HCl salt, from tert-butyl (3aR,5s,6aS)-5-(((benzyloxy)carbonyl)((tetrahydro-2H-pyran-4-yl)methyl)amino)hexahydrocyclopenta[c]pyrrole-2(1H)-carboxylate following the procedure described in Example 1, step 3. LCMS m/z=359.2 [M+H]⁺.

### 4. Synthesis of benzyl ((3aR,5s,6aS)-2-((2,4-dimethylphenyl)sulfonyl)octahydrocyclopenta[c]pyrrol-5-yl)((tetrahydro-2H-pyran-4-yl)methyl)carbamate

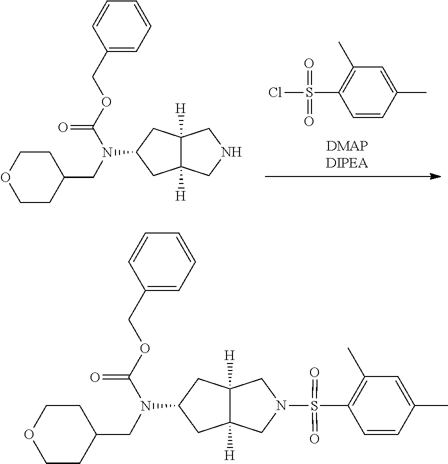

Benzyl ((3aR,5s,6aS)-2-((2,4-dimethylphenyl)sulfonyl)octahydrocyclopenta[c]pyrrol-5-yl)((tetrahydro-2H-pyran-4-yl)methyl)carbamate was obtained (45 mg, 73%) from benzyl ((3aR,5s,6aS)-octahydrocyclopenta[c]pyrrol-5-yl)((tetrahydro-2H-pyran-4-yl)methyl)carbamate and 2,4-dimethylbenzenesulfonyl chloride following the procedure described in Example 1, step 4. LCMS m/z=527.2 [M+H]⁺.

### 5. Synthesis of (3aR,5s,6aS)-2-((2,4-dimethylphenyl)sulfonyl)-N-((tetrahydro-2H-pyran-4-yl)methyl)octahydrocyclopenta[c]pyrrol-5-amine

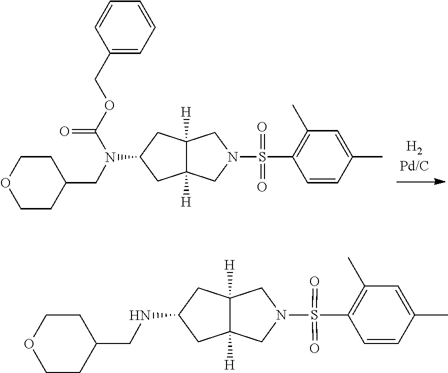 [SMILES: Cc1ccc(S(=O)(=O)N2C[C@H]3C[C@H](N(CC4CCOCC4)C(=O)OCc4ccccc4)C[C@H]3C2)c(C)c1 ; Cc1ccc(S(=O)(=O)N2C[C@H]3C[C@H](NCC4CCOCC4)C[C@H]3C2)c(C)c1]

To a solution of benzyl ((3aR,5s,6aS)-2-((2,4-dimethylphenyl)sulfonyl)octahydrocyclopenta[c]pyrrol-5-yl)((tetrahydro-2H-pyran-4-yl)methyl)carbamate (45 mg, 0.08 mmol) in MeOH (5 mL) was added 10% palladium on carbon (9 mg, 0.008 mmol). The reaction vessel was evacuated and refilled with H₂ (three times), then hydrogenated at balloon pressure of H₂ for 4 h. The reaction was filtered through Celite® and evaporated. The residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 25% (A)/75% (B) over 7.5 min (flow rate: 50 mL/min) to give (3aR,5s,6aS)-2-((2,4-dimethylphenyl)sulfonyl)-N-((tetrahydro-2H-pyran-4-yl)methyl)octahydrocyclopenta[c]pyrrol-5-amine (16 mg, 47%) as orange oil.

LCMS m/z=393.3 [M+H]⁺. t_{R}=1.43 min (TFA). t_{R}=1.99 min (NH₄OH).

### Example 8: (3aR,5s,6aS)-2-((4-(Difluoromethoxy)phenyl)sulfonyl)-N-((tetrahydro-2H-pyran-4-yl)methyl)octahydrocyclopenta[c]pyrrol-5-amine

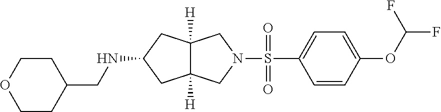 [SMILES: O=S(=O)(c1ccc(OC(F)F)cc1)N1C[C@H]2C[C@H](NCC3CCOCC3)C[C@H]2C1]

### 1. Synthesis of benzyl ((3aR,5s,6aS)-2-((4-(difluoromethoxy)phenyl)sulfonyl)octahydrocyclopenta[c]pyrrol-5-yl)((tetrahydro-2H-pyran-4-yl)methyl)carbamate

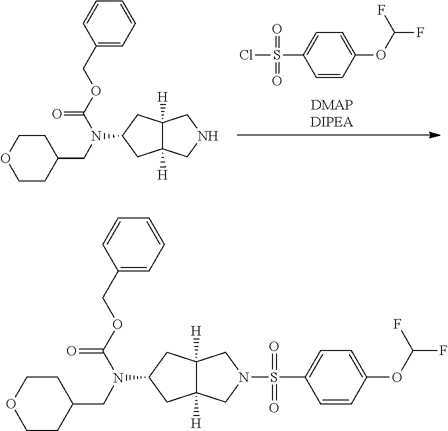 [SMILES: O=C(OCc1ccccc1)N(CC1CCOCC1)[C@@H]1C[C@H]2CNC[C@H]2C1 ; O=S(=O)(Cl)c1ccc(OC(F)F)cc1 ; O=C(OCc1ccccc1)N(CC1CCOCC1)[C@H]1C[C@@H]2CN(S(=O)(=O)c3ccc(OC(F)F)cc3)C[C@@H]2C1]

Benzyl ((3aR,5s,6aS)-2-((4-(difluoromethoxy)phenyl)sulfonyl)octahydrocyclopenta[c]pyrrol-5-yl)((tetrahydro-2H-pyran-4-yl)methyl)carbamate was obtained (46 mg, 71%) from benzyl ((3aR,5s,6aS)-octahydrocyclopenta[c]pyrrol-5-yl)((tetrahydro-2H-pyran-4-yl)methyl)carbamate (Example 7, step 3) as an HCl salt and 4-(difluoromethoxy)benzenesulfonyl chloride following the procedure described in Example 1, step 4. LCMS m/z=565.3 [M+H]⁺.

### 2. Synthesis of (3aR,5s,6aS)-2-((4-(difluoromethoxy)phenyl)sulfonyl)-N-((tetrahydro-2H-pyran-4-yl)methyl)octahydrocyclopenta[c]pyrrol-5-amine

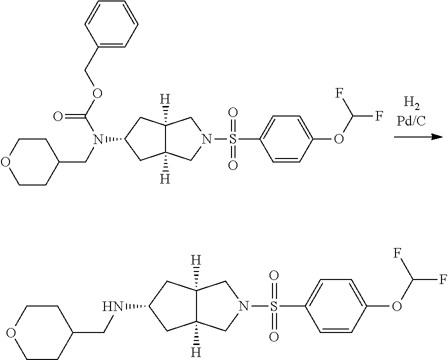 [SMILES: O=C(OCc1ccccc1)N(CC1CCOCC1)[C@H]1C[C@@H]2CN(S(=O)(=O)c3ccc(OC(F)F)cc3)C[C@@H]2C1 ; O=S(=O)(c1ccc(OC(F)F)cc1)N1C[C@H]2C[C@H](NCC3CCOCC3)C[C@H]2C1]

(3aR,5s,6aS)-2-((4-(Difluoromethoxy)phenyl)sulfonyl)-N-((tetrahydro-2H-pyran-4-yl)methyl)octahydrocyclopenta[c]pyrrol-5-amine was obtained as an orange solid (16 mg, 46%) from benzyl ((3aR,5s,6aS)-2-((4-(difluoromethoxy)phenyl)sulfonyl)octahydrocyclopenta[c]pyrrol-5-yl)((tetrahydro-2H-pyran-4-yl)methyl)carbamate following a similar procedure to that described in Example 7, step 5, except the HPLC gradient used was from 95% {H₂O}/5% {MeCN}w/0.2% NH₄OH to 30% {H₂O}/70% {MeCN}w/0.2% NH₄OH. LCMS m/z=431.2 [M+H]⁺. t_{R}=1.37 min (TFA). t_{R}=1.83 min (NH₄OH).

### Example 9: (3aR,5s,6aS)-2-((2-Methoxy-5-methylpyridin-3-yl)sulfonyl)-N-((tetrahydro-2H-pyran-4-yl)methyl)octahydrocyclopenta[c]pyrrol-5-amine

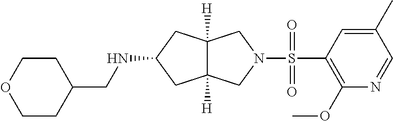 [SMILES: COc1ncc(C)cc1S(=O)(=O)N1C[C@H]2C[C@H](NCC3CCOCC3)C[C@H]2C1]

### 1. Synthesis of benzyl ((3aR,5s,6aS)-2-((2-methoxy-5-methylpyridin-3-yl)sulfonyl)octahydrocyclopenta[c]pyrrol-5-yl)((tetrahydro-2H-pyran-4-yl)methyl)carbamate

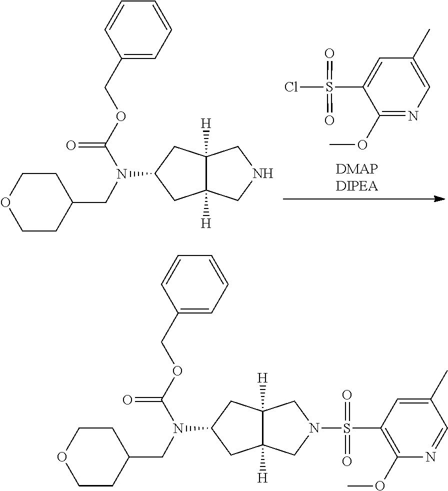

To a mixture of benzyl ((3aR,5s,6aS)-octahydrocyclopenta[c]pyrrol-5-yl)((tetrahydro-2H-pyran-4-yl)methyl)carbamate (40 mg, 0.1 mmol) (Example 7, step 3) as HCl salt in THF was added DIPEA (79 uL, 0.5 mmol) followed by DMAP (1 mg, 0.01 mmol) and then 2-methoxy-5-methylpyridine-3-sulfonyl chloride (33 mg, 0.2 mmol). After stirring at rt for 1 day, the reaction was quenched by careful addition of saturated NaHCO₃ solution. The mixture was extracted with EtOAc and the solvents were evaporated to give crude benzyl ((3aR,5s,6aS)-2-((2-methoxy-5-methylpyridin-3-yl)sulfonyl)octahydrocyclopenta[c]pyrrol-5-yl)((tetrahydro-2H-pyran-4-yl)methyl)carbamate. LCMS m/z=544.3 [M+H]⁺.

### 2. Synthesis of (3aR,5s,6aS)-2-((2-methoxy-5-methylpyridin-3-yl)sulfonyl)-N-((tetrahydro-2H-pyran-4-yl)methyl)octahydrocyclopenta[c]pyrrol-5-amine

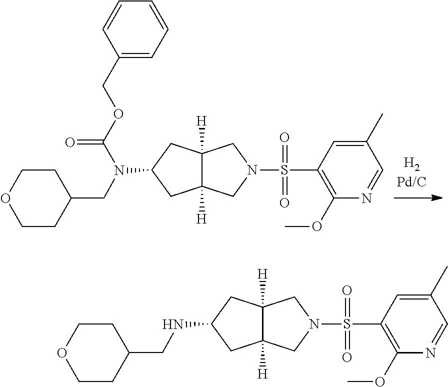 [SMILES: COc1ncc(C)cc1S(=O)(=O)N1C[C@H]2C[C@H](N(CC3CCOCC3)C(=O)OCc3ccccc3)C[C@H]2C1 ; COc1ncc(C)cc1S(=O)(=O)N1C[C@H]2C[C@H](NCC3CCOCC3)C[C@H]2C1]

(3aR,5s,6aS)-2-((2-Methoxy-5-methylpyridin-3-yl)sulfonyl)-N-((tetrahydro-2H-pyran-4-yl)methyl)octahydrocyclopenta[c]pyrrol-5-amine was obtained as an orange oil (17 mg, 40%) from benzyl ((3aR,5s,6aS)-2-((2-methoxy-5-methylpyridin-3-yl)sulfonyl)octahydrocyclopenta[c]pyrrol-5-yl)((tetrahydro-2H-pyran-4-yl)methyl)carbamate following a similar procedure to that described in Example 7, step 5, except the HPLC gradient used was from 95% {H₂O}/5% {MeCN}w/0.2% NH₄OH to 35% {H₂O}/65% {MeCN}w/0.2% NH₄OH. LCMS m/z=410.4 [M+H]⁺. t_{R}=1.24 min (TFA). t_{R}=1.75 min (NH₄OH).

### Example 10: (3aR,5s,6aS)-2-((4,6-Dimethylpyridin-3-yl)sulfonyl)-N-((tetrahydro-2H-pyran-4-yl)methyl)octahydrocyclopenta[c]pyrrol-5-amine

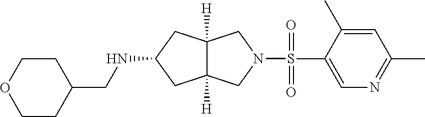 [SMILES: Cc1cc(C)c(S(=O)(=O)N2C[C@H]3C[C@H](NCC4CCOCC4)C[C@H]3C2)cn1]

### 1. Synthesis of benzyl ((3aR,5s,6aS)-2-((4,6-dimethylpyridin-3-yl)sulfonyl)octahydrocyclopenta[c]pyrrol-5-yl)((tetrahydro-2H-pyran-4-yl)methyl)carbamate

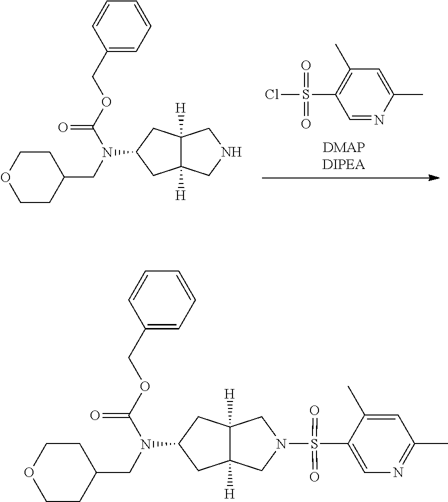 [SMILES: O=C(OCc1ccccc1)N(CC1CCOCC1)[C@@H]1C[C@H]2CNC[C@H]2C1 ; Cc1cc(C)c(S(=O)(=O)Cl)cn1 ; Cc1cc(C)c(S(=O)(=O)N2C[C@H]3C[C@H](N(CC4CCOCC4)C(=O)OCc4ccccc4)C[C@H]3C2)cn1]

Benzyl ((3aR,5s,6aS)-2-((4,6-dimethylpyridin-3-yl)sulfonyl)octahydrocyclopenta[c]pyrrol-5-yl)((tetrahydro-2H-pyran-4-yl)methyl)carbamate was obtained crude, from benzyl ((3aR,5s,6aS)-octahydrocyclopenta[c]pyrrol-5-yl)((tetrahydro-2H-pyran-4-yl)methyl)carbamate (Example 7, step 3) as HCl salt and 4,6-dimethylpyridine-3-sulfonyl chloride following the procedure described in Example 9, step 1. LCMS m/z=528.3 [M+H]⁺.

### 2. Synthesis of (3aR,5s,6aS)-2-((4,6-dimethylpyridin-3-yl)sulfonyl)-N-((tetrahydro-2H-pyran-4-yl)methyl)octahydrocyclopenta[c]pyrrol-5-amine

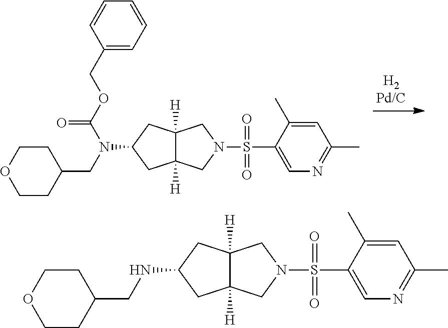 [SMILES: Cc1cc(C)c(S(=O)(=O)N2C[C@H]3C[C@H](N(CC4CCOCC4)C(=O)OCc4ccccc4)C[C@H]3C2)cn1 ; Cc1cc(C)c(S(=O)(=O)N2C[C@H]3C[C@H](NCC4CCOCC4)C[C@H]3C2)cn1]

To a solution of benzyl ((3aR,5s,6aS)-2-((4,6-dimethylpyridin-3-yl)sulfonyl)octahydrocyclopenta[c]pyrrol-5-yl)((tetrahydro-2H-pyran-4-yl)methyl)carbamate in MeOH (3 mL) was added 10% palladium on carbon (11 mg, 0.01 mmol). The reaction vessel was evacuated and refilled with H₂ (three times), then hydrogenated at balloon pressure of H₂ for 2.5 h. The reaction was filtered through Celite® and evaporated. The residue was purified by prep-HPLC (Waters SunFire Prep C18 5 μm OBD 30×100 mm; Method: (A) 95% {H₂O}//(B) 5% {MeCN}w/0.1% TFA (initial conditions hold for 0.5 min) then a linear gradient to 60% (A)/40% (B) over 7.5 min (flow rate: 50 mL/min) to give (3aR,5s,6aS)-2-((4,6-dimethylpyridin-3-yl)sulfonyl)-N-((tetrahydro-2H-pyran-4-yl)methyl)octahydrocyclopenta[c]pyrrol-5-amine (45 mg, 88%) as clear oil (TFA salt). LCMS m/z=394.4 [M+H]⁺. t_{R}=0.94 min (TFA). t_{R}=1.62 min (NH₄OH).

### Example 11: (3aR,5s,6aS)-2-((1,3-Dimethyl-1H-pyrazol-5-yl)sulfonyl)-N-((tetrahydro-2H-pyran-4-yl)methyl)octahydrocyclopenta[c]pyrrol-5-amine

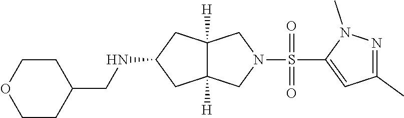 [SMILES: Cc1cc(S(=O)(=O)N2C[C@H]3C[C@H](NCC4CCOCC4)C[C@H]3C2)n(C)n1]

### 1. Synthesis of benzyl ((3aR,5s,6aS)-2-((4,6-dimethylpyridin-3-yl)sulfonyl)octahydrocyclopenta[c]pyrrol-5-yl)((tetrahydro-2H-pyran-4-yl)methyl)carbamate

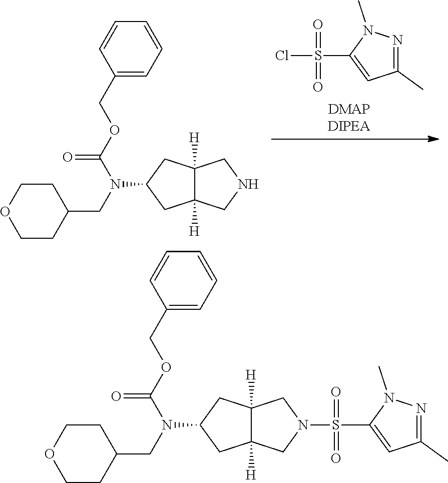

Benzyl ((3aR,5s,6aS)-2-((4,6-dimethylpyridin-3-yl)sulfonyl)octahydrocyclopenta[c]pyrrol-5-yl)((tetrahydro-2H-pyran-4-yl)methyl)carbamate was obtained crude, from benzyl ((3aR,5s,6aS)-octahydrocyclopenta[c]pyrrol-5-yl)((tetrahydro-2H-pyran-4-yl)methyl)carbamate (Example 7, step 3) as HCl salt and 1,3-dimethyl-1H-pyrazole-5-sulfonyl chloride following the procedure described in Example 9, step 1. LCMS m/z=517.2 [M+H]⁺.

### 2. Synthesis of (3aR,5s,6aS)-2-((1,3-dimethyl-1H-pyrazol-5-yl)sulfonyl)-N-((tetrahydro-2H-pyran-4-yl)methyl)octahydrocyclopenta[c]pyrrol-5-amine

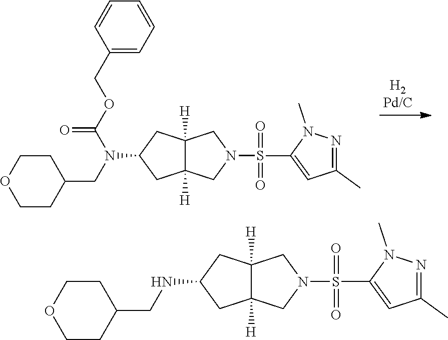

To a solution of benzyl ((3aR,5s,6aS)-2-((1,3-dimethyl-1H-pyrazol-5-yl)sulfonyl)octahydrocyclopenta[c]pyrrol-5-yl)((tetrahydro-2H-pyran-4-yl)methyl)carbamate in MeOH (3 mL) was added 10% palladium on carbon (11 mg, 0.01 mmol). The reaction vessel was evacuated and refilled with H₂ (three times), then hydrogenated at balloon pressure of H₂ for 2.5 h. Additional 10% palladium on carbon (11 mg, 0.01 mmol) was added and the reaction vessel was evacuated and refilled with H₂ (three times), then hydrogenated at balloon pressure of H₂ for an additional 2 h. The reaction was filtered through Celite® and evaporated. The residue was purified by prep-HPLC (Waters SunFire Prep C18 5 μm OBD 30×100 mm; Method: (A) 95% {H₂O}//(B) 5% {MeCN}w/0.1% TFA (initial conditions hold for 0.5 min) then a linear gradient to 50% (A)/50% (B) over 7.5 min (flow rate: 50 mL/min) to give (3aR,5s,6aS)-2-((1,3-dimethyl-1H-pyrazol-5-yl)sulfonyl)-N-((tetrahydro-2H-pyran-4-yl)methyl)octahydrocyclopenta[c]pyrrol-5-amine (20 mg, 40%) as clear oil (TFA salt). LCMS m/z=383.3 [M+H]⁺. t_{R}=1.13 min (TFA). t_{R}=1.63 min (NH₄OH).

### Example 12: (3aR,5s,6aS)-2-((3-Cyclopropyl-1-methyl-1H-pyrazol-5-yl)sulfonyl)-N-((tetrahydro-2H-pyran-4-yl)methyl)octahydrocyclopenta[c]pyrrol-5-amine

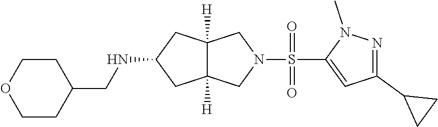 [SMILES: Cn1nc(C2CC2)cc1S(=O)(=O)N1C[C@H]2C[C@H](NCC3CCOCC3)C[C@H]2C1]

### 1. Synthesis of benzyl ((3aR,5s,6aS)-2-((3-cyclopropyl-1-methyl-1H-pyrazol-5-yl)sulfonyl)octahydrocyclopenta[c]pyrrol-5-yl)((tetrahydro-2H-pyran-4-yl)methyl)carbamate

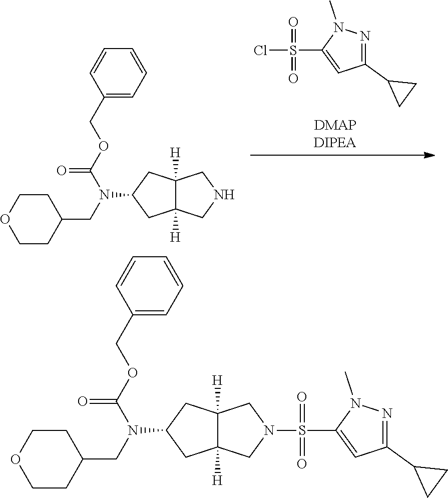

Benzyl ((3aR,5s,6aS)-2-((3-cyclopropyl-1-methyl-1H-pyrazol-5-yl)sulfonyl)octahydrocyclopenta[c]pyrrol-5-yl)((tetrahydro-2H-pyran-4-yl)methyl)carbamate was obtained crude, from benzyl ((3aR,5s,6aS)-octahydrocyclopenta[c]pyrrol-5-yl)((tetrahydro-2H-pyran-4-yl)methyl)carbamate (Example 7, step 3) as HCl salt and 3-cyclopropyl-1-methyl-1H-pyrazole-5-sulfonyl chloride following the procedure described in Example 9, step 1. LCMS m/z=543.3 [M+H]⁺.

### 2. Synthesis of (3aR,5s,6aS)-2-((3-cyclopropyl-1-methyl-1H-pyrazol-5-yl)sulfonyl)-N-((tetrahydro-2H-pyran-4-yl)methyl)octahydrocyclopenta[c]pyrrol-5-amine

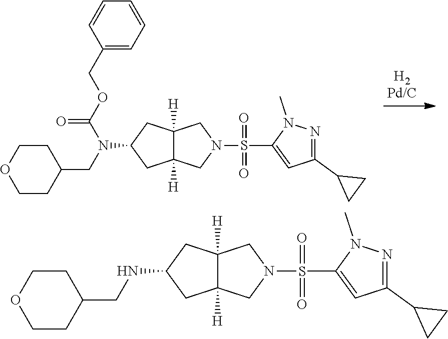

To a solution of benzyl ((3aR,5s,6aS)-2-((3-cyclopropyl-1-methyl-1H-pyrazol-5-yl)sulfonyl)octahydrocyclopenta[c]pyrrol-5-yl)((tetrahydro-2H-pyran-4-yl)methyl)carbamate in MeOH (3 mL) was added 10% palladium on carbon (11 mg, 0.01 mmol). The reaction vessel was evacuated and refilled with H₂ (three times), then hydrogenated at balloon pressure of H₂ for 2.5 h. Additional 10% palladium on carbon (11 mg, 0.01 mmol) was added and the reaction vessel was evacuated and refilled with H₂ (three times), then hydrogenated at balloon pressure of H₂ for an additional 2 h. The reaction was filtered through Celite® and evaporated. The residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 30% (A)/70% (B) over 7.5 min (flow rate: 50 mL/min) to give (3aR,5s,6aS)-2-((3-cyclopropyl-1-methyl-1H-pyrazol-5-yl)sulfonyl)-N-((tetrahydro-2H-pyran-4-yl)methyl)octahydrocyclopenta[c]pyrrol-5-amine (13 mg, 31%) as tan solid. LCMS m/z=409.4 [M+H]⁺. t_{R}=1.33 min (TFA). t_{R}=1.84 min (NH₄OH).

### Example 13: 4-((3aR,5r,6aS)-2-((4-(Difluoromethoxy)phenyl)sulfonyl)octahydrocyclopenta[c]pyrrol-5-yl)morpholine

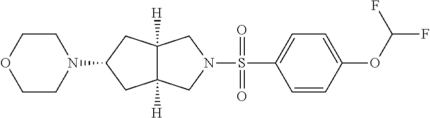 [SMILES: O=S(=O)(c1ccc(OC(F)F)cc1)N1C[C@H]2C[C@H](N3CCOCC3)C[C@H]2C1]

### 1. Synthesis of tert-butyl (3aR,5s,6aS)-5-(((benzyloxy)carbonyl)amino)hexahydrocyclopenta[c]pyrrole-2(1H)-carboxylate

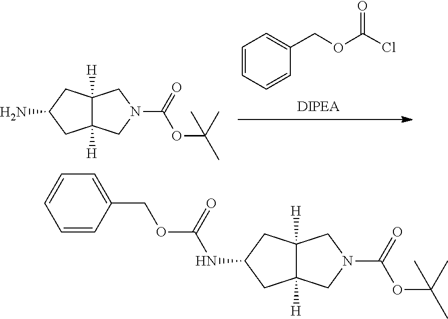

To a solution of tert-butyl (3aR,5s,6aS)-5-aminohexahydrocyclopenta[c]pyrrole-2(1H)-carboxylate (400 mg, 1.8 mmol) in DCM (10 mL) was added DIPEA (1.1 mL, 6.2 mmol) followed by benzyl chloroformate (884 μL, 2.7 mmol) as 3 M solution in toluene. After stirring at rt for 30 min, the reaction was quenched by careful addition of saturated NaHCO₃ solution. The mixture was extracted with DCM, dried over magnesium sulfate and filtered. The organics were evaporated and the residue was purified by silica gel chromatography (0-100% 3:1 EtOAc/EtOH with 2% NH₄OH in heptanes) to give tert-butyl (3aR,5s,6aS)-5-(((benzyloxy)carbonyl)amino)hexahydrocyclopenta[c]pyrrole-2(1H)-carboxylate (529 mg, 83%). LCMS m/z=383.2 [M+Na]⁺.

### 2. Synthesis of benzyl ((3aR,5s,6aS)-octahydrocyclopenta[c]pyrrol-5-yl)carbamate

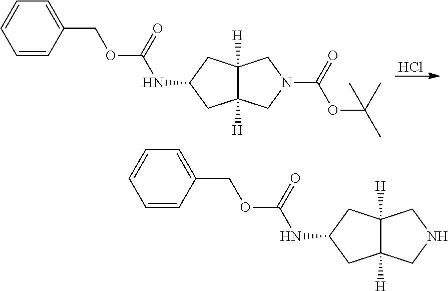 [SMILES: CC(C)(C)OC(=O)N1C[C@H]2C[C@H](NC(=O)OCc3ccccc3)C[C@H]2C1 ; Cl ; O=C(N[C@@H]1C[C@H]2CNC[C@H]2C1)OCc1ccccc1]

To a solution of tert-butyl (3aR,5s,6aS)-5-(((benzyloxy)carbonyl)amino)hexahydrocyclopenta[c]pyrrole-2(1H)-carboxylate (200 mg, 0.6 mmol) in DCM (4 mL) was added HCl (1.4 mL, 5.6 mmol) as 4 M dioxane solution. After stirring at rt for 1 h, the reaction was evaporated to dryness to give benzyl ((3aR,5s,6aS)-octahydrocyclopenta[c]pyrrol-5-yl)carbamate as HCl salt that was used without further purification. LCMS m/z=261.1 [M+H]⁺.

### 3. Synthesis of benzyl ((3aR,5s,6aS)-2-((4-(difluoromethoxy)phenyl)sulfonyl)octahydrocyclopenta[c]pyrrol-5-yl)((tetrahydro-2H-pyran-4-yl)methyl)carbamate

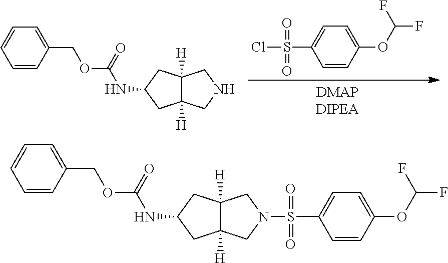 [SMILES: O=C(N[C@@H]1C[C@H]2CNC[C@H]2C1)OCc1ccccc1 ; O=S(=O)(Cl)c1ccc(OC(F)F)cc1 ; O=C(N[C@H]1C[C@@H]2CN(S(=O)(=O)c3ccc(OC(F)F)cc3)C[C@@H]2C1)OCc1ccccc1]

To a mixture of benzyl ((3aR,5s,6aS)-octahydrocyclopenta[c]pyrrol-5-yl)carbamate as HCl salt in THF (4 mL) was added DIPEA (483 μL, 2.8 mmol) followed by DMAP (7 mg, 0.06 mmol) and then 4-(difluoromethoxy)benzenesulfonyl chloride (133 μL, 0.8 mmol). After stirring at rt for 1.25 h, the reaction was quenched by careful addition of saturated NaHCO₃ solution. The mixture was extracted with EtOAc, washed with saturated sodium chloride, dried over sodium sulfate, and filtered. The solvents were evaporated and the residue was purified by silica gel chromatography (0-100% 3:1 EtOAc/EtOH with 2% NH₄OH in heptanes) to give benzyl ((3aR,5s,6aS)-2-((4-(difluoromethoxy)phenyl)sulfonyl)octahydrocyclopenta[c]pyrrol-5-yl)carbamate (230 mg, 89%). LCMS m/z=467.1 [M+H]⁺.

### 4. Synthesis of (3aR,5s,6aS)-2-((4-(difluoromethoxy)phenyl)sulfonyl)octahydrocyclopenta[c]pyrrol-5-amine

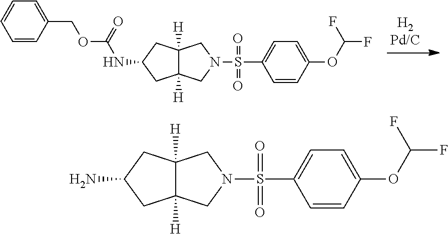

To a mixture of benzyl ((3aR,5s,6aS)-2-((4-(difluoromethoxy)phenyl)sulfonyl)octahydrocyclopenta[c]pyrrol-5-yl)carbamate (230 mg, 0.5 mmol) in EtOAc (1 mL) was added 10% palladium on carbon (52 mg, 0.05 mmol). The reaction vessel was evacuated and refilled with H₂ (three times), then hydrogenated at balloon pressure of H₂ for 1 day. The reaction was filtered through Celite® and evaporated. The residue was taken up in MeOH (10 mL) and to this was added 10% palladium on carbon (52 mg, 0.05 mmol). The reaction vessel was evacuated and refilled with H₂ (three times), then hydrogenated at balloon pressure of H₂ for 2 h. The reaction was filtered through Celite® and evaporated to give crude (3aR,5s,6aS)-2-((4-(difluoromethoxy)phenyl)sulfonyl)octahydrocyclopenta[c]pyrrol-5-amine (164 mg, assumed 100%). LCMS m/z=333.1 [M+H]⁺.

### 5. Synthesis of 4-((3aR,5r,6aS)-2-((4-(difluoromethoxy)phenyl)sulfonyl)octahydrocyclopenta[c]pyrrol-5-yl)morpholine

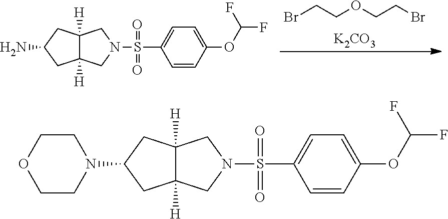

To a mixture of (3aR,5s,6aS)-2-((4-(difluoromethoxy)phenyl)sulfonyl)octahydrocyclopenta[c]pyrrol-5-amine (41 mg, 0.1 mmol) and 1-bromo-2-(2-bromoethoxy)ethane (23 μL, 0.2 mmol) in MeCN (1.5 mL) was added K₂CO₃ (60 mg, 0.4 mmol). The reaction was heated at 90° C. for 1 day, then cooled and filtered. The residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 35% (A)/65% (B) over 7.5 min (flow rate: 50 mL/min) to give 4-((3aR,5r,6aS)-2-((4-(difluoromethoxy)phenyl)sulfonyl)octahydrocyclopenta[c]pyrrol-5-yl)morpholine (30 mg, 61%) as white solid. LCMS m/z=403.2 [M+H]⁺. t_{R}=1.29 min (TFA). t_{R}^{=1.66} min (NH₄OH).

### Example 14: (3aR,5s,6aS)-2-((4-(Difluoromethoxy)phenyl)sulfonyl)-N-(oxetan-3-yl)octahydrocyclopenta[c]pyrrol-5-amine

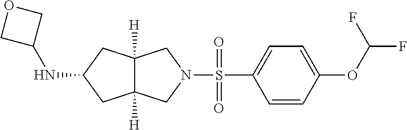 [SMILES: O=S(=O)(c1ccc(OC(F)F)cc1)N1C[C@H]2C[C@H](NC3COC3)C[C@H]2C1]

### 1. Synthesis of (3aR,5s,6aS)-2-((4-(difluoromethoxy)phenyl)sulfonyl)-N-(oxetan-3-yl)octahydrocyclopenta[c]pyrrol-5-amine

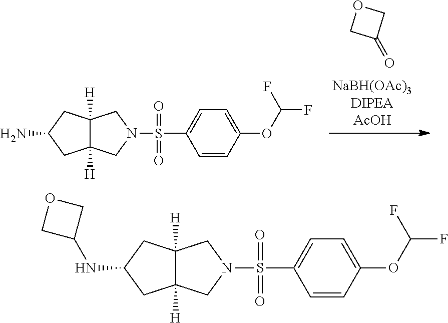

To a mixture of (3aR,5s,6aS)-2-((4-(difluoromethoxy)phenyl)sulfonyl)octahydrocyclopenta[c]pyrrol-5-amine (41 mg, 0.1 mmol) (Example 13, step 4) and oxetan-3-one (10 μL, 0.2 mmol) in DCM (2 mL) was added DIPEA (43 μL, 0.2 mmol) with stirring. After 15 min, acetic acid (21 μL, 0.4 mmol) was added with stirring. After an additional 15 min, sodium triacetoxyborohydride (104 mg, 0.5 mmol) was added with stirring. The reaction was stirred for 1 day at rt, then was quenched by slow addition of saturated NaHCO₃ solution. The mixture was extracted with EtOAc and the solvents were evaporated. The residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 40% (A)/60% (B) over 7.5 min (flow rate: 50 mL/min) to give (3aR,5s,6aS)-2-((4-(difluoromethoxy)phenyl)sulfonyl)-N-(oxetan-3-yl)octahydrocyclopenta[c]pyrrol-5-amine (19 mg, 40%) as white solid. LCMS m/z=389.2 [M+H]⁺. t_{R}=1.26 min (TFA). t_{R}=1.53 min (NH₄OH).

### Example 15: (3aR,5s,6aS)-2-((4-(Difluoromethoxy)phenyl)sulfonyl)-N-(tetrahydrofuran-3-yl)octahydrocyclopenta[c]pyrrol-5-amine

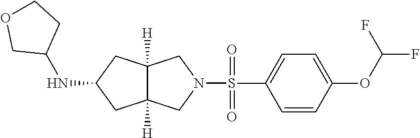 [SMILES: O=S(=O)(c1ccc(OC(F)F)cc1)N1C[C@H]2C[C@H](NC3CCOC3)C[C@H]2C1]

### 1. Synthesis of (3aR,5s,6aS)-2-((4-(difluoromethoxy)phenyl)sulfonyl)-N-(tetrahydrofuran-3-yl)octahydrocyclopenta[c]pyrrol-5-amine

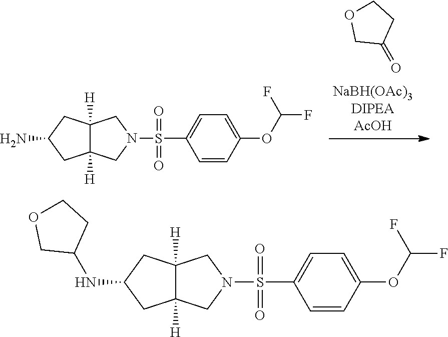

(3aR,5s,6aS)-2-((4-(Difluoromethoxy)phenyl)sulfonyl)-N-(tetrahydrofuran-3-yl)octahydrocyclopenta[c]pyrrol-5-amine was obtained as a white solid (23 mg, 47%) from (3aR,5s,6aS)-2-((4-(difluoromethoxy)phenyl)sulfonyl)octahydrocyclopenta[c]pyrrol-5-amine (Example 13, step 4) and dihydrofuran-3(2H)-one, following a similar procedure to that described in Example 14, except the HPLC gradient used was from 95% {H₂O}/5% {MeCN}w/0.2% NH₄OH to 35% {H₂O}/65% {MeCN}w/0.2% NH₄OH. LCMS m/z=403.2 [M+H]⁺. t_{R}=1.29 min (TFA). t_{R}=1.62 min (NH₄OH).

### Example 16: (3aR,5s,6aS)-2-((4-(Difluoromethoxy)phenyl)sulfonyl)-N-(tetrahydro-2H-pyran-4-yl)octahydrocyclopenta[c]pyrrol-5-amine

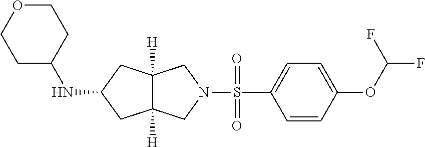 [SMILES: O=S(=O)(c1ccc(OC(F)F)cc1)N1C[C@H]2C[C@H](NC3CCOCC3)C[C@H]2C1]

### 1. Synthesis of (3aR,5s,6aS)-2-((4-(difluoromethoxy)phenyl)sulfonyl)-N-(tetrahydro-2H-pyran-4-yl)octahydrocyclopenta[c]pyrrol-5-amine

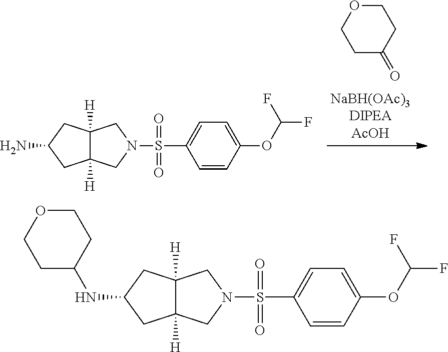

(3aR,5s,6aS)-2-((4-(Difluoromethoxy)phenyl)sulfonyl)-N-(tetrahydro-2H-pyran-4-yl)octahydrocyclopenta[c]pyrrol-5-amine was obtained as a white solid (23 mg, 45%) from (3aR,5s,6aS)-2-((4-(difluoromethoxy)phenyl)sulfonyl)octahydrocyclopenta[c]pyrrol-5-amine (Example 13, step 4) and tetrahydro-4H-pyran-4-one, following the procedure described in Example 15. LCMS m/z=417.2 [M+H]⁺. t_{R}=1.33 min (TFA). t_{R}=1.69 min (NH₄OH).

### Example 17: (3aR,5s,6aS)-2-((4-(Difluoromethoxy)phenyl)sulfonyl)-N-methyl-N-((tetrahydro-2H-pyran-4-yl)methyl)octahydrocyclopenta[c]pyrrol-5-amine

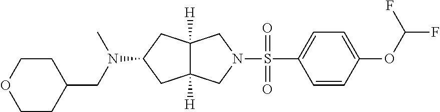 [SMILES: CN(CC1CCOCC1)[C@H]1C[C@@H]2CN(S(=O)(=O)c3ccc(OC(F)F)cc3)C[C@@H]2C1]

### 1. Synthesis of tert-butyl (3aR,5s,6aS)-5-(methyl((tetrahydro-2H-pyran-4-yl)methyl)amino)hexahydrocyclopenta[c]pyrrole-2(1H)-carboxylate

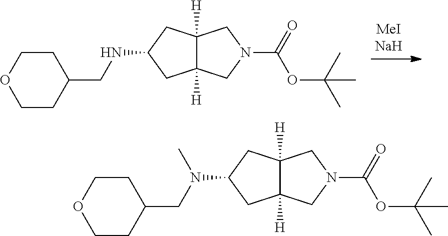 [SMILES: CC(C)(C)OC(=O)N1C[C@H]2C[C@H](NCC3CCOCC3)C[C@H]2C1 ; CN(CC1CCOCC1)[C@H]1C[C@@H]2CN(C(=O)OC(C)(C)C)C[C@@H]2C1]

To a solution of tert-butyl (3aR,5s,6aS)-5-(((tetrahydro-2H-pyran-4-yl)methyl)amino)hexahydrocyclopenta[c]pyrrole-2(1H)-carboxylate (78 mg, 0.2 mmol) (Example 7, step 1) in DMF (2 mL) was added 60% NaH (19 mg, 0.5 mmol) followed by iodomethane (16 μL, 0.3 mmol). The reaction was stirred for 2 h at rt then was quenched by addition of saturated NaHCO₃ solution. The mixture was extracted with EtOAc, dried over sodium sulfate, filtered and evaporated. The residue was purified by SCX (eluting with 2N NH₃ in MeOH) to give crude tert-butyl (3aR,5s,6aS)-5-(methyl((tetrahydro-2H-pyran-4-yl)methyl)amino)hexahydrocyclopenta[c]pyrrole-2(1H)-carboxylate (73 mg, 91%). LCMS m/z=339.2 [M+H]⁺.

### 2. Synthesis of (3aR,5s,6aS)-N-methyl-N-((tetrahydro-2H-pyran-4-yl)methyl)octahydrocyclopenta[c]pyrrol-5-amine

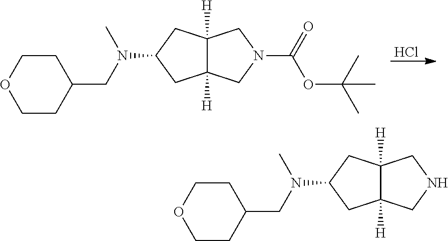

To a solution of tert-butyl (3aR,5s,6aS)-5-(methyl((tetrahydro-2H-pyran-4-yl)methyl)amino)hexahydrocyclopenta[c]pyrrole-2(1H)-carboxylate (73 mg, crude) in DCM (2 mL) was added HCl (543 μL, 2.2 mmol) as 4 M solution in dioxane. The reaction was stirred for 1 day at rt. The reaction was evaporated to dryness to give crude (3aR,5s,6aS)-N-methyl-N-((tetrahydro-2H-pyran-4-yl)methyl)octahydrocyclopenta[c]pyrrol-5-amine as presumed bis-HCl salt. LCMS m/z=239.2 [M+H]⁺.

### 3. Synthesis of (3aR,5s,6aS)-2-((4-(difluoromethoxy)phenyl)sulfonyl)-N-methyl-N-((tetrahydro-2H-pyran-4-yl)methyl)octahydrocyclopenta[c]pyrrol-5-amine

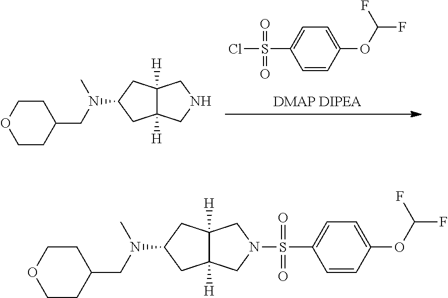 [SMILES: CN(CC1CCOCC1)[C@@H]1C[C@H]2CNC[C@H]2C1 ; O=S(=O)(Cl)c1ccc(OC(F)F)cc1 ; CN(CC1CCOCC1)[C@H]1C[C@@H]2CN(S(=O)(=O)c3ccc(OC(F)F)cc3)C[C@@H]2C1]

To a mixture of (3aR,5s,6aS)-N-methyl-N-((tetrahydro-2H-pyran-4-yl)methyl)octahydrocyclopenta[c]pyrrol-5-amine (assumed 34 mg, 0.1 mmol) as bis-HCl salt\in THF (1 mL) was added DIPEA (110 μL, 0.6 mmol) followed by DMAP (1 mg, 0.01 mmol) and then 4-(difluoromethoxy)benzenesulfonyl chloride (40 μL, 0.3 mmol). The reaction was stirred at rt for 3.5 h, then the reaction was quenched by careful addition of saturated NaHCO₃ solution. The mixture was extracted with EtOAc. The solvents were evaporated and the residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 25% (A)/75% (B) over 7.5 min (flow rate: 50 mL/min) to give (3aR,5s,6aS)-2-((4-(difluoromethoxy)phenyl)sulfonyl)-N-methyl-N-((tetrahydro-2H-pyran-4-yl)methyl)octahydrocyclopenta[c]pyrrol-5-amine (14 mg, 28%) as white solid. LCMS m/z=445.3 [M+H]⁺. t_{R}=1.45 min (TFA). t_{R}=2.18 min (NH₄OH).

### Example 18: (3aR,5s,6aS)-2-((3-Cyclopropyl-1-methyl-1H-pyrazol-5-yl)sulfonyl)-N-methyl-N-((tetrahydro-2H-pyran-4-yl)methyl)octahydrocyclopenta[c]pyrrol-5-amine

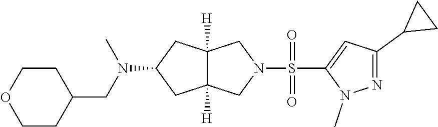 [SMILES: CN(CC1CCOCC1)[C@H]1C[C@@H]2CN(S(=O)(=O)c3cc(C4CC4)nn3C)C[C@@H]2C1]

### 1. Synthesis of (3aR,5s,6aS)-2-((3-cyclopropyl-1-methyl-1H-pyrazol-5-yl)sulfonyl)-N-methyl-N-((tetrahydro-2H-pyran-4-yl)methyl)octahydrocyclopenta[c]pyrrol-5-amine

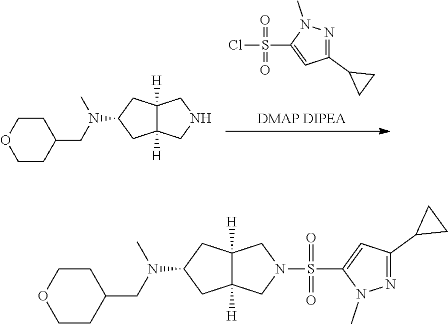 [SMILES: CN(CC1CCOCC1)[C@@H]1C[C@H]2CNC[C@H]2C1 ; Cn1nc(C2CC2)cc1S(=O)(=O)Cl ; CN(CC1CCOCC1)[C@H]1C[C@@H]2CN(S(=O)(=O)c3cc(C4CC4)nn3C)C[C@@H]2C1]

To a mixture of (3aR,5s,6aS)-N-methyl-N-((tetrahydro-2H-pyran-4-yl)methyl)octahydrocyclopenta[c]pyrrol-5-amine (assumed 34 mg, 0.1 mmol), (Example 17, step 2) as bis-HCl salt in THF (1 mL) was added DIPEA (110 μL, 0.6 mmol) followed by DMAP (1 mg, 0.01 mmol) and then 3-cyclopropyl-1-methyl-1H-pyrazole-5-sulfonyl chloride (55 mg, 0.3 mmol). The reaction was stirred at rt for 3.5 h, then was quenched by careful addition of saturated NaHCO₃ solution. The mixture was extracted with EtOAc. The solvents were evaporated and the residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 30% (A)/70% (B) over 7.5 min (flow rate: 50 mL/min) to give (3aR,5s,6aS)-2-((3-cyclopropyl-1-methyl-1H-pyrazol-5-yl)sulfonyl)-N-methyl-N-((tetrahydro-2H-pyran-4-yl)methyl)octahydrocyclopenta[c]pyrrol-5-amine (12 mg, 23%) as white solid. LCMS m/z=423.4 [M+H]⁺. t_{R}=1.37 min (TFA). t_{R}=2.13 min (NH₄OH).

### Example 19: (3aR,5s,6aS)-2-((4-(Difluoromethoxy)phenyl)sulfonyl)-N-(2-oxaspiro[3.3]heptan-6-yl)octahydrocyclopenta[c]pyrrol-5-amine

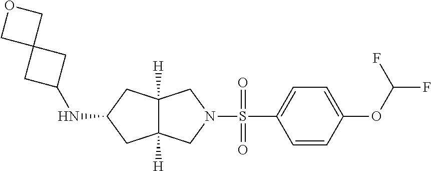 [SMILES: O=S(=O)(c1ccc(OC(F)F)cc1)N1C[C@H]2C[C@H](NC3CC4(COC4)C3)C[C@H]2C1]

### 1. Synthesis of (3aR,5s,6aS)-2-((4-(difluoromethoxy)phenyl)sulfonyl)-N-(2-oxaspiro[3.3]heptan-6-yl)octahydrocyclopenta[c]pyrrol-5-amine

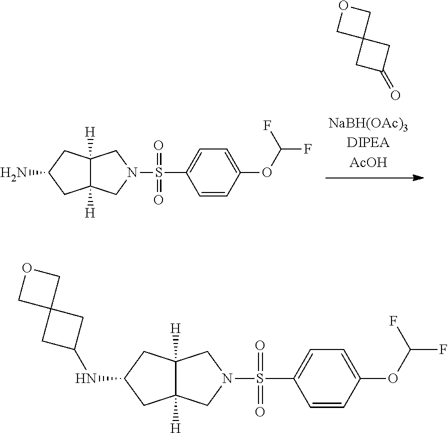 [SMILES: O=C1CC2(COC2)C1 ; N[C@H]1C[C@@H]2CN(S(=O)(=O)c3ccc(OC(F)F)cc3)C[C@@H]2C1 ; O=S(=O)(c1ccc(OC(F)F)cc1)N1C[C@H]2C[C@H](NC3CC4(COC4)C3)C[C@H]2C1]

(3aR,5s,6aS)-2-((4-(Difluoromethoxy)phenyl)sulfonyl)-N-(2-oxaspiro[3.3]heptan-6-yl)octahydrocyclopenta[c]pyrrol-5-amine was obtained as a yellow oil (17 mg, 45%) from (3aR,5s,6aS)-2-((4-(difluoromethoxy)phenyl)sulfonyl)octahydrocyclopenta[c]pyrrol-5-amine (Example 13, step 4) and 2-oxaspiro[3.3]heptan-6-one following the procedure described in Example 15. LCMS m/z=429.2 [M+H]⁺. t_{R}=1.40 min (TFA). t_{R}=1.83 min (NH₄OH).

### Example 20: (3aR,5s,6aS)-2-((3-Cyclopropyl-1-methyl-1H-pyrazol-5-yl)sulfonyl)-N-(2-oxaspiro[3.3]heptan-6-yl)octahydrocyclopenta[c]pyrrol-5-amine

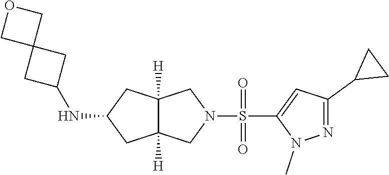 [SMILES: Cn1nc(C2CC2)cc1S(=O)(=O)N1C[C@H]2C[C@H](NC3CC4(COC4)C3)C[C@H]2C1]

### 1. Synthesis of benzyl ((3aR,5s,6aS)-2-((3-cyclopropyl-1-methyl-1H-pyrazol-5-yl)sulfonyl)octahydrocyclopenta[c]pyrrol-5-yl)carbamate

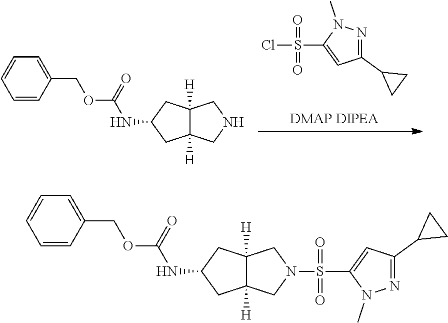 [SMILES: O=C(N[C@@H]1C[C@H]2CNC[C@H]2C1)OCc1ccccc1 ; Cn1nc(C2CC2)cc1S(=O)(=O)Cl ; Cn1nc(C2CC2)cc1S(=O)(=O)N1C[C@H]2C[C@H](NC(=O)OCc3ccccc3)C[C@H]2C1]

Benzyl ((3aR,5s,6aS)-2-((3-cyclopropyl-1-methyl-1H-pyrazol-5-yl)sulfonyl)octahydrocyclopenta[c]pyrrol-5-yl)carbamate was obtained (141 mg, 89%) from benzyl ((3aR,5s,6aS)-octahydrocyclopenta[c]pyrrol-5-yl)carbamate (Example 13, step 2) as HCl salt and 3-cyclopropyl-1-methyl-1H-pyrazole-5-sulfonyl chloride following the procedure described in Example 13, step 3. LCMS m/z=445.2 [M+H]⁺.

### 2. Synthesis of (3aR,5s,6aS)-2-((3-cyclopropyl-1-methyl-1H-pyrazol-5-yl)sulfonyl)octahydrocyclopenta[c]pyrrol-5-amine

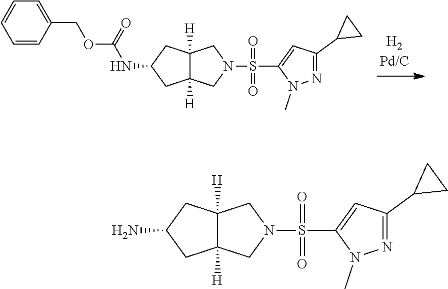 [SMILES: Cn1nc(C2CC2)cc1S(=O)(=O)N1C[C@H]2C[C@H](NC(=O)OCc3ccccc3)C[C@H]2C1 ; Cn1nc(C2CC2)cc1S(=O)(=O)N1C[C@H]2C[C@H](N)C[C@H]2C1]

To a mixture of benzyl ((3aR,5s,6aS)-2-((3-cyclopropyl-1-methyl-1H-pyrazol-5-yl)sulfonyl)octahydrocyclopenta[c]pyrrol-5-yl)carbamate (141 mg, 0.3 mmol) in EtOAc (10 mL) was added 10% palladium on carbon (34 mg, 0.03 mmol). The reaction vessel was evacuated and refilled with H₂ (three times), then hydrogenated at balloon pressure of H₂ for 1 day. The reaction was filtered through Celite® and evaporated to give crude (3aR,5s,6aS)-2-((3-cyclopropyl-1-methyl-1H-pyrazol-5-yl)sulfonyl)octahydrocyclopenta[c]pyrrol-5-amine (88 mg, 90%). LCMS m/z=311.1 [M+H]⁺.

### 3. Synthesis of (3aR,5s,6aS)-2-((3-cyclopropyl-1-methyl-1H-pyrazol-5-yl)sulfonyl)-N-(2-oxaspiro[3.3]heptan-6-yl)octahydrocyclopenta[c]pyrrol-5-amine

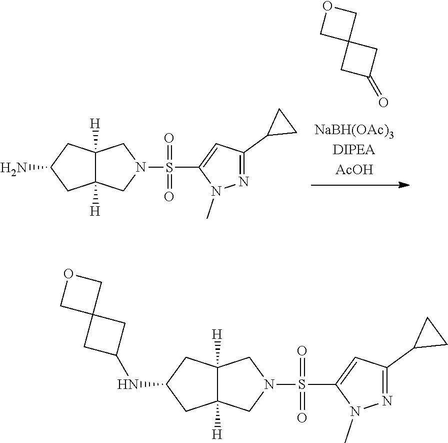 [SMILES: O=C1CC2(COC2)C1 ; Cn1nc(C2CC2)cc1S(=O)(=O)N1C[C@H]2C[C@H](N)C[C@H]2C1 ; Cn1nc(C2CC2)cc1S(=O)(=O)N1C[C@H]2C[C@H](NC3CC4(COC4)C3)C[C@H]2C1]

To a mixture of (3aR,5s,6aS)-2-((3-cyclopropyl-1-methyl-1H-pyrazol-5-yl)sulfonyl)octahydrocyclopenta[c]pyrrol-5-amine (22 mg, 0.07 mmol) and 2-oxaspiro[3.3]heptan-6-one (9 mg, 0.08 mmol) in DCM (1 mL) was added DIPEA (32 μL, 0.2 mmol) with stirring. After 15 min, acetic acid (10 μL, 0.2 mmol) was added with stirring. After an additional 15 min, sodium triacetoxyborohydride (60 mg, 0.3 mmol) was added with stirring. The reaction was stirred for 2.5 h at rt, then was quenched by slow addition of saturated NaHCO₃ solution. The mixture was extracted with EtOAc and the solvents were evaporated. The residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 35% (A)/65% (B) over 7.5 min (flow rate: 50 mL/min) to give (3aR,5s,6aS)-2-((3-cyclopropyl-1-methyl-1H-pyrazol-5-yl)sulfonyl)-N-(2-oxaspiro[3.3]heptan-6-yl)octahydrocyclopenta[c]pyrrol-5-amine (12 mg, 43%) as white solid.

LCMS m/z=407.3 [M+H]⁺. t_{R}=1.45 min (TFA). t_{R}=1.71 min (NH₄OH).

### Example 21: (3aR,5s,6aS)-2-((3-Cyclopropyl-1-methyl-1H-pyrazol-5-yl)sulfonyl)-N-((3-methyloxetan-3-yl)methyl)octahydrocyclopenta[c]pyrrol-5-amine

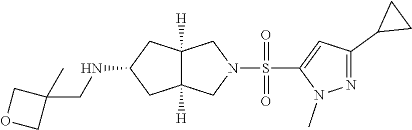 [SMILES: Cn1nc(C2CC2)cc1S(=O)(=O)N1C[C@H]2C[C@H](NCC3(C)COC3)C[C@H]2C1]

### 1. Synthesis of (3aR,5s,6aS)-2-((3-cyclopropyl-1-methyl-1H-pyrazol-5-yl)sulfonyl)-N-((3-methyloxetan-3-yl)methyl)octahydrocyclopenta[c]pyrrol-5-amine

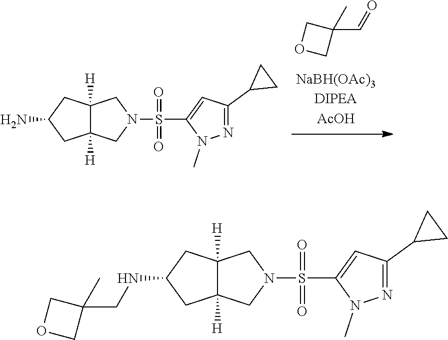

(3aR,5s,6aS)-2-((3-Cyclopropyl-1-methyl-1H-pyrazol-5-yl)sulfonyl)-N-((3-methyloxetan-3-yl)methyl)octahydrocyclopenta[c]pyrrol-5-amine was obtained as a white solid (13 mg, 48%) from (3aR,5s,6aS)-2-((3-cyclopropyl-1-methyl-1H-pyrazol-5-yl)sulfonyl)octahydrocyclopenta[c]pyrrol-5-amine (Example 20, step 2) and 3-methyloxetane-3-carbaldehyde following the procedure described in Example 20, step 3. LCMS m/z=395.3 [M+H]⁺. t_{R}=1.44 min (TFA). t_{R}=1.78 min (NH₄OH).

### Example 22: (3aR,5s,6aS)-2-((3-Cyclopropyl-1-methyl-1H-pyrazol-5-yl)sulfonyl)-N-(tetrahydro-2H-pyran-4-yl)octahydrocyclopenta[c]pyrrol-5-amine

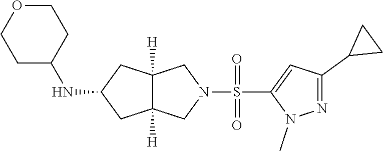 [SMILES: Cn1nc(C2CC2)cc1S(=O)(=O)N1C[C@H]2C[C@H](NC3CCOCC3)C[C@H]2C1]

### 1. Synthesis of benzyl ((3aR,5s,6aS)-2-((3-cyclopropyl-1-methyl-1H-pyrazol-5-yl)sulfonyl)octahydrocyclopenta[c]pyrrol-5-yl)carbamate

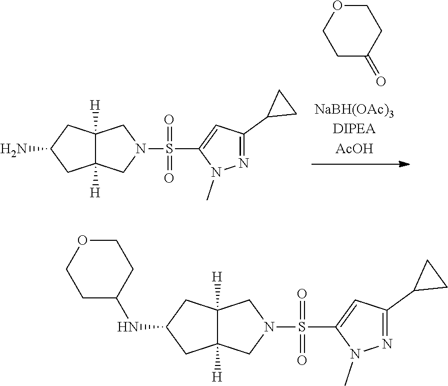 [SMILES: O=C1CCOCC1 ; Cn1nc(C2CC2)cc1S(=O)(=O)N1C[C@H]2C[C@H](N)C[C@H]2C1 ; Cn1nc(C2CC2)cc1S(=O)(=O)N1C[C@H]2C[C@H](NC3CCOCC3)C[C@H]2C1]

To a mixture of (3aR,5s,6aS)-2-((3-cyclopropyl-1-methyl-1H-pyrazol-5-yl)sulfonyl)octahydrocyclopenta[c]pyrrol-5-amine (22 mg, 0.07 mmol) (Example 20, step 2) and tetrahydro-4H-pyran-4-one (7 μL, 0.08 mmol) in DCM (2 mL) was added DIPEA (32 μL, 0.2 mmol) with stirring. After 15 min, acetic acid (11 μL, 0.2 mmol) was added with stirring. After an additional 15 min, sodium triacetoxyborohydride (60 mg, 0.3 mmol) was added with stirring. The reaction was stirred for 1 h at rt, then was quenched by slow addition of saturated NaHCO₃ solution. The mixture was extracted with EtOAc and the solvents were evaporated. The residue was purified by prep-HPLC (Waters SunFire Prep C18 5 μm OBD 30×100 mm; Method: (A) 95% {H₂O}//(B) 5% {MeCN}w/0.1% TFA (initial conditions hold for 0.5 min) then a linear gradient to 45% (A)/55% (B) over 7.5 min (flow rate: 50 mL/min) to give (3aR,5s,6aS)-2-((3-cyclopropyl-1-methyl-1H-pyrazol-5-yl)sulfonyl)-N-(tetrahydro-2H-pyran-4-yl)octahydrocyclopenta[c]pyrrol-5-amine (11 mg, 30%) as white solid (TFA salt). LCMS m/z=395.3 [M+H]⁺. t_{R}=1.45 min (TFA). t_{R}=1.74 min (NH₄OH).

### Example 23: (3aR,5s,6aS)-N-Methyl-2-((2-methyl-6-(trifluoromethyl)pyridin-3-yl)sulfonyl)-N-((tetrahydro-2H-pyran-4-yl)methyl)octahydrocyclopenta[c]pyrrol-5-amine

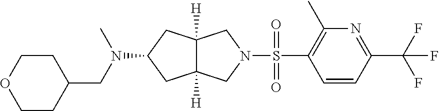 [SMILES: Cc1nc(C(F)(F)F)ccc1S(=O)(=O)N1C[C@H]2C[C@H](N(C)CC3CCOCC3)C[C@H]2C1]

### 1. Synthesis of tert-butyl (3aR,5s,6aS)-5-(((benzyloxy)carbonyl)(methyl)amino)hexahydrocyclopenta[c]pyrrole-2(1H)-carboxylate

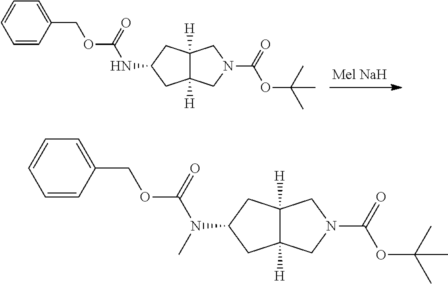 [SMILES: CC(C)(C)OC(=O)N1C[C@H]2C[C@H](NC(=O)OCc3ccccc3)C[C@H]2C1 ; CN(C(=O)OCc1ccccc1)[C@H]1C[C@@H]2CN(C(=O)OC(C)(C)C)C[C@@H]2C1]

To a solution of tert-butyl (3aR,5s,6aS)-5-(((benzyloxy)carbonyl)amino)hexahydrocyclopenta[c]pyrrole-2(1H)-carboxylate (123 mg, 0.3 mmol) (Example 13, step 1) in DMF (3 mL) was added 60% NaH (109 mg, 2.7 mmol). The suspension was stirred for 10 min then iodomethane (53 μL, 0.9 mmol) was added. The reaction was stirred for 1 h at rt, then was quenched by addition of saturated ammonium chloride solution. The mixture was extracted with EtOAc, washed with saturated sodium chloride, dried over sodium sulfate, filtered and evaporated. The residue was purified by silica gel chromatography (0-100% 3:1 EtOAc/EtOH with 2% NH₄OH in heptanes) to give tert-butyl (3aR,5s,6aS)-5-(((benzyloxy)carbonyl)(methyl)amino)hexahydrocyclopenta[c]pyrrole-2(1H)-carboxylate (109 mg, 85%). LCMS m/z=397.3 [M+Na]⁺.

### 2. Synthesis of benzyl methyl((3aR,5s,6aS)-octahydrocyclopenta[c]pyrrol-5-yl)carbamate

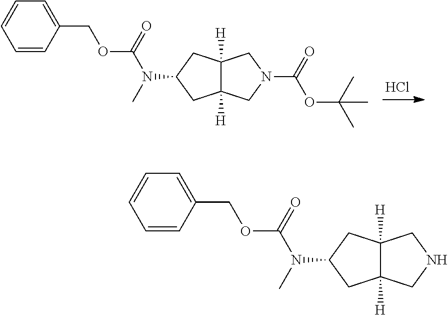 [SMILES: CN(C(=O)OCc1ccccc1)[C@H]1C[C@@H]2CN(C(=O)OC(C)(C)C)C[C@@H]2C1 ; Cl ; CN(C(=O)OCc1ccccc1)[C@@H]1C[C@H]2CNC[C@H]2C1]

To a solution of tert-butyl (3aR,5s,6aS)-5-(((benzyloxy)carbonyl)(methyl)amino)hexahydrocyclopenta[c]pyrrole-2(1H)-carboxylate (109 mg, 0.3 mmol) in DCM (2.5 mL) was added HCl (728 μL, 2.9 mmol) as 4 M solution in dioxane. The reaction was stirred for 2 h at rt. The reaction was evaporated to dryness to give crude benzyl methyl((3aR,5s,6aS)-octahydrocyclopenta[c]pyrrol-5-yl)carbamate as HCl salt.

LCMS m/z=275.2 [M+H]⁺.

### 3. Synthesis of benzyl methyl((3aR,5s,6aS)-2-((2-methyl-6-(trifluoromethyl)pyridin-3-yl)sulfonyl)octahydrocyclopenta[c]pyrrol-5-yl)carbamate

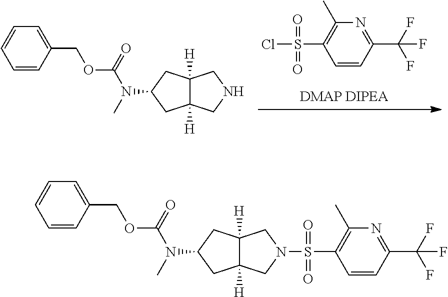 [SMILES: CN(C(=O)OCc1ccccc1)[C@@H]1C[C@H]2CNC[C@H]2C1 ; Cc1nc(C(F)(F)F)ccc1S(=O)(=O)Cl ; Cc1nc(C(F)(F)F)ccc1S(=O)(=O)N1C[C@H]2C[C@H](N(C)C(=O)OCc3ccccc3)C[C@H]2C1]

To a mixture of benzyl methyl((3aR,5s,6aS)-octahydrocyclopenta[c]pyrrol-5-yl)carbamate as HCl salt in DCM (2.5 mL) was added DIPEA (157 μL, 0.9 mmol) followed by 2-methyl-6-(trifluoromethyl)pyridine-3-sulfonyl chloride (101 mg, 0.4 mmol). The reaction was stirred at rt for 1 day, then the reaction was quenched by careful addition of saturated NaHCO₃ solution. The mixture was extracted with DCM and the combined organic extracts dried over magnesium sulfate and filtered. The solvents were evaporated and the residue was purified by silica gel chromatography (0-100% EtOAc in heptanes) to give benzyl methyl((3aR,5s,6aS)-2-((2-methyl-6-(trifluoromethyl)pyridin-3-yl)sulfonyl)octahydrocyclopenta[c]pyrrol-5-yl)carbamate (132 mg, 92%). LCMS m/z=498.2 [M+H]⁺.

### 4. Synthesis of (3aR,5s,6aS)-N-methyl-2-((2-methyl-6-(trifluoromethyl)pyridin-3-yl)sulfonyl)octahydrocyclopenta[c]pyrrol-5-amine

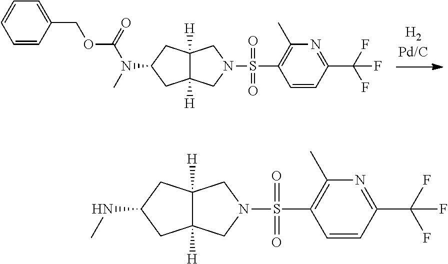 [SMILES: Cc1nc(C(F)(F)F)ccc1S(=O)(=O)N1C[C@H]2C[C@H](N(C)C(=O)OCc3ccccc3)C[C@H]2C1 ; CN[C@H]1C[C@@H]2CN(S(=O)(=O)c3ccc(C(F)(F)F)nc3C)C[C@@H]2C1]

(3aR,5s,6aS)-N-Methyl-2-((2-methyl-6-(trifluoromethyl)pyridin-3-yl)sulfonyl)octahydrocyclopenta[c]pyrrol-5-amine was obtained, 97 mg, quantitative, from benzyl methyl((3aR,5s,6aS)-2-((2-methyl-6-(trifluoromethyl)pyridin-3-yl)sulfonyl)octahydrocyclopenta[c]pyrrol-5-yl)carbamate, following the procedure described in Example 13, step 4. LCMS m/z=364.2 [M+H]⁺.

### 5. Synthesis of (3aR,5s,6aS)-N-methyl-2-((2-methyl-6-(trifluoromethyl)pyridin-3-yl)sulfonyl)-N-((tetrahydro-2H-pyran-4-yl)methyl)octahydrocyclopenta[c]pyrrol-5-amine

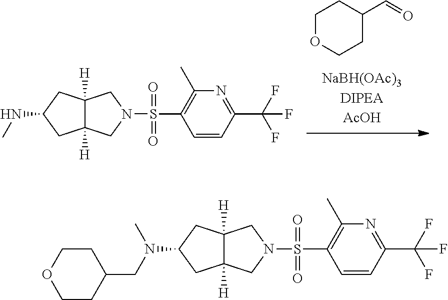 [SMILES: O=CC1CCOCC1 ; CN[C@H]1C[C@@H]2CN(S(=O)(=O)c3ccc(C(F)(F)F)nc3C)C[C@@H]2C1 ; Cc1nc(C(F)(F)F)ccc1S(=O)(=O)N1C[C@H]2C[C@H](N(C)CC3CCOCC3)C[C@H]2C1]

To a mixture of (3aR,5s,6aS)-N-methyl-2-((2-methyl-6-(trifluoromethyl)pyridin-3-yl)sulfonyl)octahydrocyclopenta[c]pyrrol-5-amine (24 mg, 0.07 mmol) and tetrahydro-2H-pyran-4-carbaldehyde (12 mg, 0.1 mmol) in DCM (1 mL) was added DIPEA (35 μL, 0.2 mmol) with stirring. After 15 min, acetic acid (11 μL, 0.2 mmol) was added with stirring. After an additional 15 min, sodium triacetoxyborohydride (56 mg, 0.3 mmol) was added with stirring. The reaction was stirred for 1 day at rt, then was quenched by slow addition of saturated NaHCO₃ solution. The mixture was extracted with EtOAc and the solvents were evaporated. The residue was purified by prep-HPLC (Waters SunFire Prep C18 5 μm OBD 30×100 mm; Method: (A) 95% {H₂O}//(B) 5% {MeCN}w/0.1% TFA (initial conditions hold for 0.5 min) then a linear gradient to 40% (A)/60% (B) over 7.5 min (flow rate: 50 mL/min) to give (3aR,5s,6aS)-N-methyl-2-((2-methyl-6-(trifluoromethyl)pyridin-3-yl)sulfonyl)-N-((tetrahydro-2H-pyran-4-yl)methyl)octahydrocyclopenta[c]pyrrol-5-amine (23 mg, 62%) as yellow oil (TFA salt). LCMS m/z=462.3 [M+H]⁺. t_{R}=1.57 min (TFA). t_{R}=2.24 min (NH₄OH).

### Example 24: (3aR,5s,6aS)-N-Methyl-2-((2-methyl-6-(trifluoromethyl)pyridin-3-yl)sulfonyl)-N-(tetrahydro-2H-pyran-4-yl)octahydrocyclopenta[c]pyrrol-5-amine

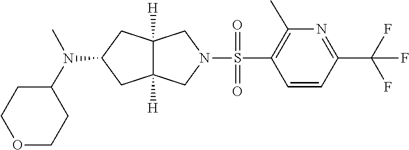 [SMILES: Cc1nc(C(F)(F)F)ccc1S(=O)(=O)N1C[C@H]2C[C@H](N(C)C3CCOCC3)C[C@H]2C1]

### 1. Synthesis of (3aR,5s,6aS)-N-methyl-2-((2-methyl-6-(trifluoromethyl)pyridin-3-yl)sulfonyl)-N-(tetrahydro-2H-pyran-4-yl)octahydrocyclopenta[c]pyrrol-5-amine

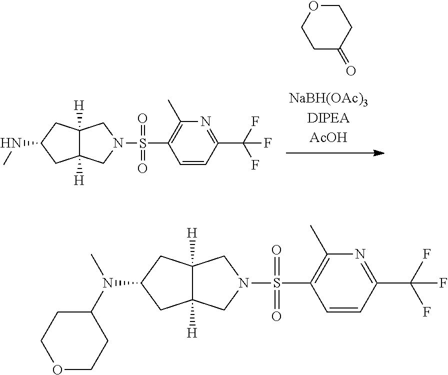 [SMILES: O=C1CCOCC1 ; CN[C@H]1C[C@@H]2CN(S(=O)(=O)c3ccc(C(F)(F)F)nc3C)C[C@@H]2C1 ; Cc1nc(C(F)(F)F)ccc1S(=O)(=O)N1C[C@H]2C[C@H](N(C)C3CCOCC3)C[C@H]2C1]

(3aR,5s,6aS)-N-Methyl-2-((2-methyl-6-(trifluoromethyl)pyridin-3-yl)sulfonyl)-N-(tetrahydro-2H-pyran-4-yl)octahydrocyclopenta[c]pyrrol-5-amine was obtained as a yellow oil, as a TFA salt (6 mg, 16%) from (3aR,5s,6aS)-N-methyl-2-((2-methyl-6-(trifluoromethyl)pyridin-3-yl)sulfonyl)octahydrocyclopenta[c]pyrrol-5-amine (Example 23, step 4), and tetrahydro-4H-pyran-4-one, following the procedure described in Example 23, step 5. LCMS m/z=448.3 [M+H]⁺. t_{R}=1.54 min (TFA). t_{R}=1.94 min (NH₄OH).

### Example 25: (3aR,5s,6aS)-N-Methyl-2-((2-methyl-6-(trifluoromethyl)pyridin-3-yl)sulfonyl)-N-(2-oxaspiro[3.3]heptan-6-yl)octahydrocyclopenta[c]pyrrol-5-amine

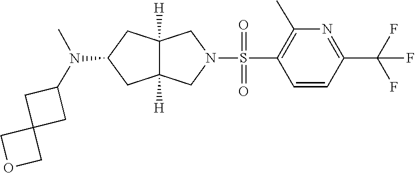 [SMILES: Cc1nc(C(F)(F)F)ccc1S(=O)(=O)N1C[C@H]2C[C@H](N(C)C3CC4(COC4)C3)C[C@H]2C1]

### 1. Synthesis of (3aR,5s,6aS)-N-methyl-2-((2-methyl-6-(trifluoromethyl)pyridin-3-yl)sulfonyl)-N-(2-oxaspiro[3.3]heptan-6-yl)octahydrocyclopenta[c]pyrrol-5-amine

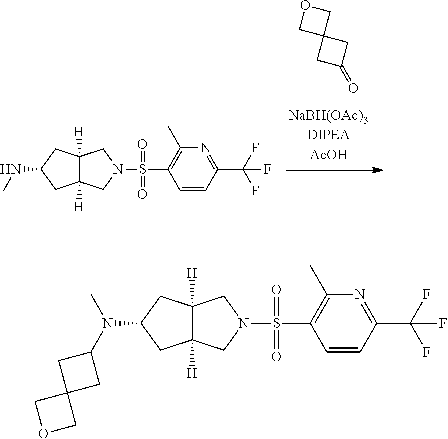 [SMILES: O=C1CC2(COC2)C1 ; CN[C@H]1C[C@@H]2CN(S(=O)(=O)c3ccc(C(F)(F)F)nc3C)C[C@@H]2C1 ; Cc1nc(C(F)(F)F)ccc1S(=O)(=O)N1C[C@H]2C[C@H](N(C)C3CC4(COC4)C3)C[C@H]2C1]

To a mixture of (3aR,5s,6aS)-N-methyl-2-((2-methyl-6-(trifluoromethyl)pyridin-3-yl)sulfonyl)octahydrocyclopenta[c]pyrrol-5-amine (24 mg, 0.07 mmol) (Example 23, step 4) and 2-oxaspiro[3.3]heptan-6-one (12 mg, 0.1 mmol) in DCM (1 mL) was added DIPEA (35 μL, 0.2 mmol) with stirring. After 15 min, acetic acid (11 μL, 0.2 mmol) was added with stirring. After an additional 15 min, sodium triacetoxyborohydride (56 mg, 0.3 mmol) was added with stirring. The reaction was stirred for 1 day at rt, then was quenched by slow addition of saturated NaHCO₃ solution. The mixture was extracted with EtOAc and the solvents were evaporated. The residue was purified by prep-HPLC (Waters XSelect CSH Prep C18 5 μm OBD 30×100 mm; Method: (A) 95% {H₂O}//(B) 5% {MeCN}w/0.2% NH₄OH (initial conditions hold for 0.5 min) then a linear gradient to 25% (A)/75% (B) over 7.5 min (flow rate: 50 mL/min) to give (3aR,5s,6aS)-N-methyl-2-((2-methyl-6-(trifluoromethyl)pyridin-3-yl)sulfonyl)-N-(2-oxaspiro[3.3]heptan-6-yl)octahydrocyclopenta[c]pyrrol-5-amine (13 mg, 41%) as yellow solid. LCMS m/z=460.3 [M+H]⁺. t_{R}=1.52 min (TFA). t_{R}=1.94 min (NH₄OH).

### Example 26: (3aR,5s,6aS)-N-Methyl-2-((2-methyl-6-(trifluoromethyl)pyridin-3-yl)sulfonyl)-N-((3-methyloxetan-3-yl)methyl)octahydrocyclopenta[c]pyrrol-5-amine

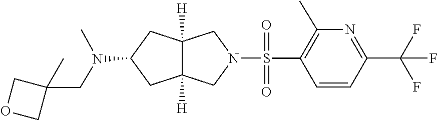 [SMILES: Cc1nc(C(F)(F)F)ccc1S(=O)(=O)N1C[C@H]2C[C@H](N(C)CC3(C)COC3)C[C@H]2C1]

### 1. Synthesis of (3aR,5s,6aS)-N-methyl-2-((2-methyl-6-(trifluoromethyl)pyridin-3-yl)sulfonyl)-N-((3-methyloxetan-3-yl)methyl)octahydrocyclopenta[c]pyrrol-5-amine

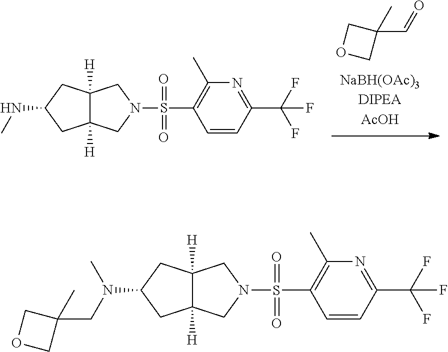

(3aR,5s,6aS)-N-Methyl-2-((2-methyl-6-(trifluoromethyl)pyridin-3-yl)sulfonyl)-N-((3-methyloxetan-3-yl)methyl)octahydrocyclopenta[c]pyrrol-5-amine was obtained as a white solid (15 mg, 51%) from (3aR,5s,6aS)-N-methyl-2-((2-methyl-6-(trifluoromethyl)pyridin-3-yl)sulfonyl)octahydrocyclopenta[c]pyrrol-5-amine (Example 23, step 4) and 3-methyloxetane-3-carbaldehyde, following a similar procedure to that described in Example 25, except the HPLC gradient used was Method: (A) 90% {H₂O}//(B) 10% {MeCN}w/0.2% NH₄OH (initial conditions hold for 0.5 min) then a linear gradient to 20% (A)/80% (B) over 7.5 min (flow rate: 50 mL/min). LCMS m/z=448.3 [M+H]⁺. t_{R}=1.50 min (TFA). t_{R}=2.14 min (NH₄OH).

### Example 27: (3aR,5s,6aS)-2-((2-Methyl-6-(trifluoromethyl)pyridin-3-yl)sulfonyl)-N-((tetrahydro-2H-pyran-4-yl)methyl)octahydrocyclopenta[c]pyrrol-5-amine

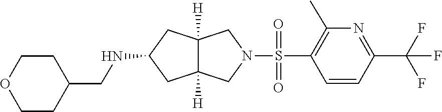 [SMILES: Cc1nc(C(F)(F)F)ccc1S(=O)(=O)N1C[C@H]2C[C@H](NCC3CCOCC3)C[C@H]2C1]

### 1. Synthesis of benzyl ((3aR,5s,6aS)-2-((2-methyl-6-(trifluoromethyl)pyridin-3-yl)sulfonyl)octahydrocyclopenta[c]pyrrol-5-yl)carbamate

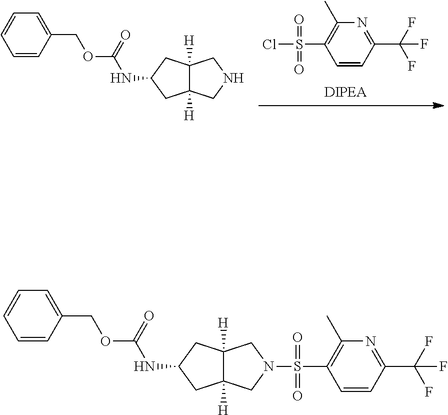 [SMILES: O=C(N[C@@H]1C[C@H]2CNC[C@H]2C1)OCc1ccccc1 ; Cc1nc(C(F)(F)F)ccc1S(=O)(=O)Cl ; Cc1nc(C(F)(F)F)ccc1S(=O)(=O)N1C[C@H]2C[C@H](NC(=O)OCc3ccccc3)C[C@H]2C1]

Benzyl ((3aR,5s,6aS)-2-((2-methyl-6-(trifluoromethyl)pyridin-3-yl)sulfonyl)octahydrocyclopenta[c]pyrrol-5-yl)carbamate was obtained (235 mg, 77%) from benzyl ((3aR,5s,6aS)-octahydrocyclopenta[c]pyrrol-5-yl)carbamate (Example 13, step 2) as HCl salt and 2-methyl-6-(trifluoromethyl)pyridine-3-sulfonyl chloride, following the method described in Example 23, step 3. LCMS m/z=484.1 [M+H]⁺.

### 2. Synthesis of (3aR,5s,6aS)-2-((2-methyl-6-(trifluoromethyl)pyridin-3-yl)sulfonyl)octahydrocyclopenta[c]pyrrol-5-amine

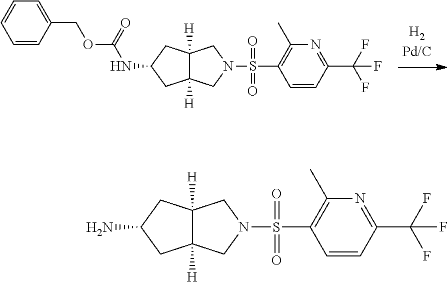 [SMILES: Cc1nc(C(F)(F)F)ccc1S(=O)(=O)N1C[C@H]2C[C@H](NC(=O)OCc3ccccc3)C[C@H]2C1 ; Cc1nc(C(F)(F)F)ccc1S(=O)(=O)N1C[C@H]2C[C@H](N)C[C@H]2C1]

To a mixture of benzyl ((3aR,5s,6aS)-2-((2-methyl-6-(trifluoromethyl)pyridin-3-yl)sulfonyl)octahydrocyclopenta[c]pyrrol-5-yl)carbamate (235 mg, 0.5 mmol) in EtOAc (10 mL) was added 10% palladium on carbon (52 mg, 0.05 mmol). The reaction vessel was evacuated and refilled with H₂ (three times), then hydrogenated at balloon pressure of H₂ for 1 day. The reaction was filtered through Celite®. To the solution was added 10% palladium on carbon (52 mg, 0.05 mmol). The reaction vessel was evacuated and refilled with H₂ (three times), then hydrogenated at balloon pressure of H₂ for 2 h. The reaction was filtered through Celite®, which was then washed with EtOAc. The solvents were evaporated to give crude (3aR,5s,6aS)-2-((2-methyl-6-(trifluoromethyl)pyridin-3-yl)sulfonyl)octahydrocyclopenta[c]pyrrol-5-amine (149 mg, 88%). LCMS m/z=350.1 [M+H]⁺.

### 3. Synthesis of (3aR,5s,6aS)-2-((2-methyl-6-(trifluoromethyl)pyridin-3-yl)sulfonyl)-N-((tetrahydro-2H-pyran-4-yl)methyl)octahydrocyclopenta[c]pyrrol-5-amine

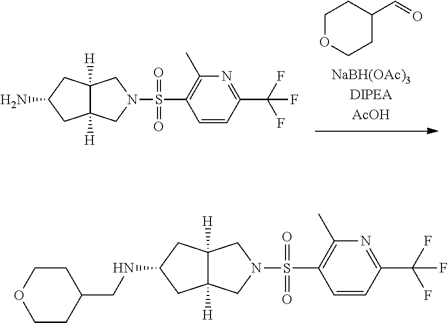 [SMILES: Cc1nc(C(F)(F)F)ccc1S(=O)(=O)N1C[C@H]2C[C@H](N)C[C@H]2C1 ; O=CC1CCOCC1 ; Cc1nc(C(F)(F)F)ccc1S(=O)(=O)N1C[C@H]2C[C@H](NCC3CCOCC3)C[C@H]2C1]

(3aR,5s,6aS)-2-((2-Methyl-6-(trifluoromethyl)pyridin-3-yl)sulfonyl)-N-((tetrahydro-2H-pyran-4-yl)methyl)octahydrocyclopenta[c]pyrrol-5-amine was obtained as an off-white solid (10 mg, 58%) from (3aR,5s,6aS)-2-((2-methyl-6-(trifluoromethyl)pyridin-3-yl)sulfonyl)octahydrocyclopenta[c]pyrrol-5-amine and tetrahydro-2H-pyran-4-carbaldehyde, following the procedure described in Example 25. LCMS m/z=448.1 [M+H]⁺. t_{R}=1.70 min (TFA). t_{R}^{=2.21} min (NH₄OH).

### Example 28: (3aR,5s,6aS)-2-((2-Methyl-6-(trifluoromethyl)pyridin-3-yl)sulfonyl)-N-(tetrahydro-2H-pyran-4-yl)octahydrocyclopenta[c]pyrrol-5-amine

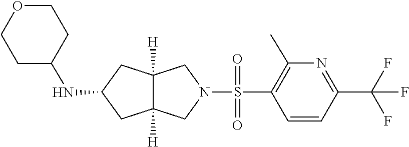 [SMILES: Cc1nc(C(F)(F)F)ccc1S(=O)(=O)N1C[C@H]2C[C@H](NC3CCOCC3)C[C@H]2C1]

### 1. Synthesis of (3aR,5s,6aS)-2-((2-methyl-6-(trifluoromethyl)pyridin-3-yl)sulfonyl)-N-(tetrahydro-2H-pyran-4-yl)octahydrocyclopenta[c]pyrrol-5-amine

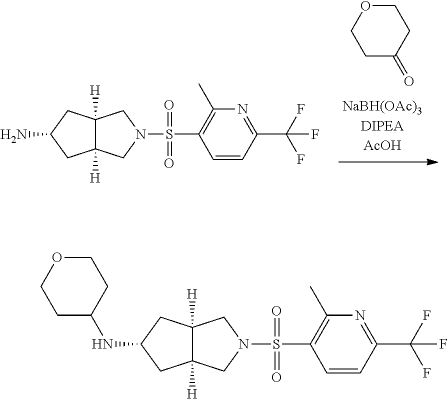 [SMILES: O=C1CCOCC1 ; Cc1nc(C(F)(F)F)ccc1S(=O)(=O)N1C[C@H]2C[C@H](N)C[C@H]2C1 ; Cc1nc(C(F)(F)F)ccc1S(=O)(=O)N1C[C@H]2C[C@H](NC3CCOCC3)C[C@H]2C1]

(3aR,5s,6aS)-2-((2-Methyl-6-(trifluoromethyl)pyridin-3-yl)sulfonyl)-N-(tetrahydro-2H-pyran-4-yl)octahydrocyclopenta[c]pyrrol-5-amine was obtained as a white solid (11 mg, 67%) from (3aR,5s,6aS)-2-((2-methyl-6-(trifluoromethyl)pyridin-3-yl)sulfonyl)octahydrocyclopenta[c]pyrrol-5-amine (Example 27, step 2) and tetrahydro-4H-pyran-4-one, following a similar procedure to that described in Example 25, except the HPLC gradient used was from 95% {H₂O}/5% {MeCN}w/0.2% NH₄OH to 30% {H₂O}/70% {MeCN}w/0.2% NH₄OH. LCMS m/z=434.1 [M+H]⁺. t_{R}=1.66 min (TFA). t_{R}=2.04 min (NH₄OH).

### Example 29: (3aR,5s,6aS)-2-((2-Methyl-6-(trifluoromethyl)pyridin-3-yl)sulfonyl)-N-((3-methyloxetan-3-yl)methyl)octahydrocyclopenta[c]pyrrol-5-amine

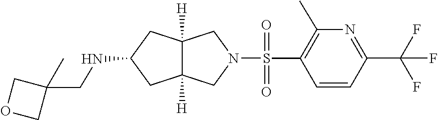 [SMILES: Cc1nc(C(F)(F)F)ccc1S(=O)(=O)N1C[C@H]2C[C@H](NCC3(C)COC3)C[C@H]2C1]

### 1. Synthesis of (3aR,5s,6aS)-2-((2-methyl-6-(trifluoromethyl)pyridin-3-yl)sulfonyl)-N-((3-methyloxetan-3-yl)methyl)octahydrocyclopenta[c]pyrrol-5-amine

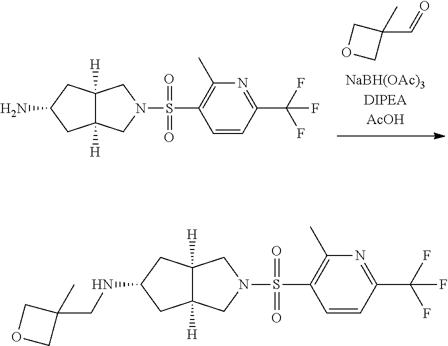

(3aR,5s,6aS)-2-((2-Methyl-6-(trifluoromethyl)pyridin-3-yl)sulfonyl)-N-((3-methyloxetan-3-yl)methyl)octahydrocyclopenta[c]pyrrol-5-amine was obtained as a white solid (18 mg, 48%) from (3aR,5s,6aS)-2-((2-methyl-6-(trifluoromethyl)pyridin-3-yl)sulfonyl)octahydrocyclopenta[c]pyrrol-5-amine (Example 27, step 2) and 3-methyloxetane-3-carbaldehyde following the method described in Example 25. LCMS m/z=434.3 [M+H]⁺. t_{R}=1.50 min (TFA). t_{R}=1.93 min (NH₄OH).

### Example 30: (3aR,5s,6aS)-2-((2-Methyl-6-(trifluoromethyl)pyridin-3-yl)sulfonyl)-N-(2-oxaspiro[3.3]heptan-6-yl)octahydrocyclopenta[c]pyrrol-5-amine

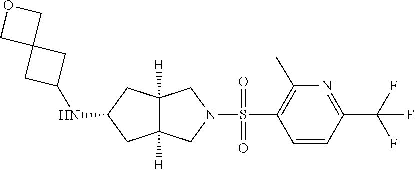 [SMILES: Cc1nc(C(F)(F)F)ccc1S(=O)(=O)N1C[C@H]2C[C@H](NC3CC4(COC4)C3)C[C@H]2C1]

### 1. Synthesis of (3aR,5s,6aS)-2-((2-methyl-6-(trifluoromethyl)pyridin-3-yl)sulfonyl)-N-(2-oxaspiro[3.3]heptan-6-yl)octahydrocyclopenta[c]pyrrol-5-amine

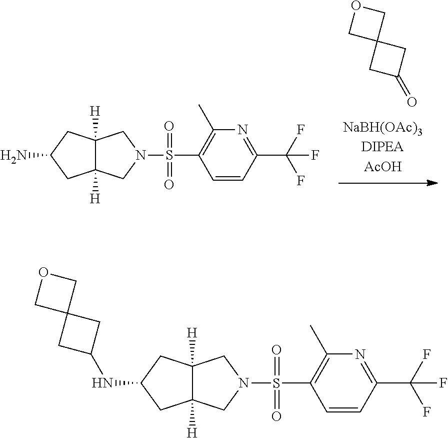 [SMILES: O=C1CC2(COC2)C1 ; Cc1nc(C(F)(F)F)ccc1S(=O)(=O)N1C[C@H]2C[C@H](N)C[C@H]2C1 ; Cc1nc(C(F)(F)F)ccc1S(=O)(=O)N1C[C@H]2C[C@H](NC3CC4(COC4)C3)C[C@H]2C1]

(3aR,5s,6aS)-2-((2-Methyl-6-(trifluoromethyl)pyridin-3-yl)sulfonyl)-N-(2-oxaspiro[3.3]heptan-6-yl)octahydrocyclopenta[c]pyrrol-5-amine was obtained as a yellow oil (23 mg, 61%) from (3aR,5s,6aS)-2-((2-methyl-6-(trifluoromethyl)pyridin-3-yl)sulfonyl)octahydrocyclopenta[c]pyrrol-5-amine (Example 27, step 2) and 2-oxaspiro[3.3]heptan-6-one following a similar procedure to that described in Example 25, except the HPLC gradient used was from 95% {H₂O}/5% {MeCN}w/0.2% NH₄OH to 30% {H₂O}/70% {MeCN}w/0.2% NH₄OH. LCMS m/z=446.3 [M+H]⁺. t_{R}=1.49 min (TFA). t_{R}=1.78 min (NH₄OH).

### Example 31: (3aR,6aS)-2-((1,3-dimethyl-1H-pyrazol-5-yl)sulfonyl)-5-(tetrahydro-2H-pyran-4-yl)octahydropyrrolo[3,4-c]pyrrole

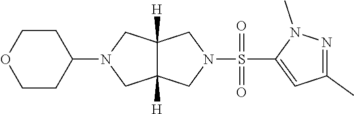 [SMILES: Cc1cc(S(=O)(=O)N2C[C@H]3CN(C4CCOCC4)C[C@H]3C2)n(C)n1]

### 1. Synthesis of tert-butyl (3aR,6aS)-5-((1,3-dimethyl-1H-pyrazol-5-yl)sulfonyl)hexahydropyrrolo[3,4-c]pyrrole-2(1H)-carboxylate

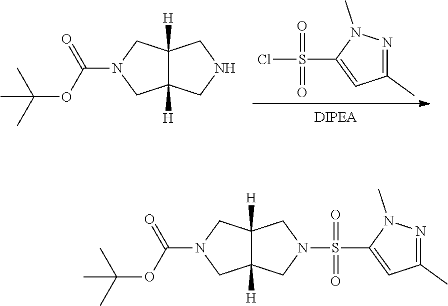 [SMILES: CC(C)(C)OC(=O)N1C[C@H]2CNC[C@H]2C1 ; Cc1cc(S(=O)(=O)Cl)n(C)n1 ; Cc1cc(S(=O)(=O)N2C[C@H]3CN(C(=O)OC(C)(C)C)C[C@H]3C2)n(C)n1]

To a mixture of tert-butyl (3aR,6aS)-hexahydropyrrolo[3,4-c]pyrrole-2(1H)-carboxylate (156 mg, 0.73 mmol) and 2,5-dimethylpyrazole-3-sulfonyl chloride (136 mg, 0.70 mmol) in DCM (4 mL) was added DIPEA (0.24 μL, 1.40 mmol). The reaction mixture was stirred at rt overnight. The mixture was washed with saturated NaHCO₃, water, and concentrated. The crude material was purified by silica gel chromatography (20-100% EtOAc in heptane) to afford tert-butyl (3aR,6aS)-5-((1,3-dimethyl-1H-pyrazol-5-yl)sulfonyl)hexahydropyrrolo[3,4-c]pyrrole-2(1H)-carboxylate (225 mg, 87%) as a white solid. LCMS m/z=393.2 [M+Na]⁺. ¹H-NMR (400 MHz, MeOH-d₄): δ (ppm) 6.64-6.56 (m, 1H), 4.02 (s, 3H), 3.61-3.41 (m, 4H), 3.18 (br dd, J=10.2, 3.6 Hz, 4H), 3.00-2.85 (m, 2H), 2.25 (s, 3H), 1.45 (s, 9H).

### 2. Synthesis of (3aR,6aS)-2-((1,3-dimethyl-1H-pyrazol-5-yl)sulfonyl)octahydropyrrolo[3,4-c]pyrrole

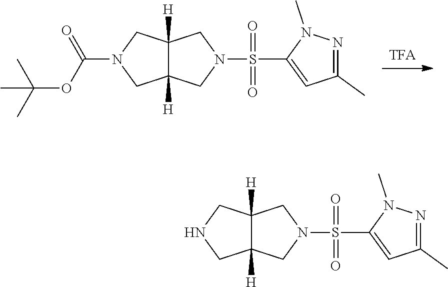 [SMILES: Cc1cc(S(=O)(=O)N2C[C@H]3CN(C(=O)OC(C)(C)C)C[C@H]3C2)n(C)n1 ; Cc1cc(S(=O)(=O)N2C[C@H]3CNC[C@H]3C2)n(C)n1]

To a reaction via containing tert-butyl 3aR,6aS)-5-((1,3-dimethyl-1H-pyrazol-5-yl)sulfonyl)hexahydropyrrolo[3,4-c]pyrrole-2(1H)-carboxylate (215 mg, 0.58 mmol) in 1,1,1,3,3,3-hexafluoropropan-2-ol (3 mL) was added trifluoroacetic acid (89 μL, 1.16 mmol) dropwise at rt and the reaction was stirred at rt overnight. The reaction was concentrated under reduced pressure and co-evaporated with MeCN (×3) to give (3aR,6aS)-2-((1,3-dimethyl-1H-pyrazol-5-yl)sulfonyl)octahydropyrrolo[3,4-c]pyrrole, (TFA salt) as white solid (296 mg, 100%). LCMS m/z=271.1 [M+H]⁺.

### 3. Synthesis of (3aR,6aS)-2-((1,3-dimethyl-1H-pyrazol-5-yl)sulfonyl)-5-(tetrahydro-2H-pyran-4-yl)octahydropyrrolo[3,4-c]pyrrole

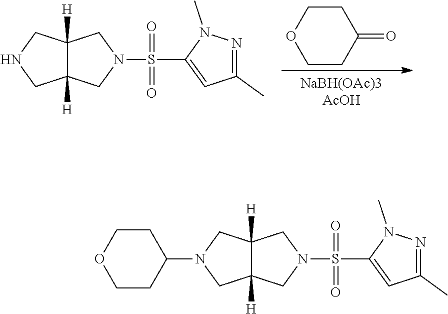 [SMILES: Cc1cc(S(=O)(=O)N2C[C@H]3CNC[C@H]3C2)n(C)n1 ; O=C1CCOCC1 ; Cc1cc(S(=O)(=O)N2C[C@H]3CN(C4CCOCC4)C[C@H]3C2)n(C)n1]

To a mixture of tetrahydro-4H-pyran-4-one (14 mg, 0.14 mmol) and (3aR,6aS)-2-((1,3-dimethyl-1H-pyrazol-5-yl)sulfonyl)octahydropyrrolo[3,4-c]pyrrole, as TFA salt (47 mg, 0.093 mmol) in DCM (2 mL) was added DIPEA (48 μL, 0.28 mmol). The mixture was stirred at rt for 5 min, acetic acid (16 μL, 0.28 mmol) added, and the solution stirred for another 5 min. Sodium triacetoxyborohydride (78 mg, 0.37 mmol) was added in one portion and the reaction was stirred at rt overnight. The reaction was quenched with saturated NaHCO₃, diluted with DCM, and stirred for 5 min. The aqueous layer was removed, the organic phase was washed with water and concentrated. The residue was purified by silica gel column (EtOAc/EtOH 3/1) to give (3aR,6aS)-2-((1,3-dimethyl-1H-pyrazol-5-yl)sulfonyl)-5-(tetrahydro-2H-pyran-4-yl)octahydropyrrolo[3,4-c]pyrrole (21 mg, 64%) as a white solid. LCMS m/z=355.2 [M+H]⁺. ¹H-NMR (400 MHz, MeOH-d₄): δ (ppm) 6.59 (s, 1H), 4.02 (s, 3H), 3.95-3.87 (m, 2H), 3.40 (td, J=11.8, 2.3 Hz, 2H), 3.27-3.19 (m, 2H), 3.18-3.11 (m, 2H), 2.93 (dd, J=9.5, 7.5 Hz, 2H), 2.87-2.75 (m, 2H), 2.34-2.20 (m, 6H), 1.87-1.74 (m, 2H), 1.58-1.41 (m, 2H).

### Example 32: 2-(Mesitylsulfonyl)-5-(tetrahydro-2H-pyran-4-yl)octahydropyrrolo[3,4-c]pyrrole

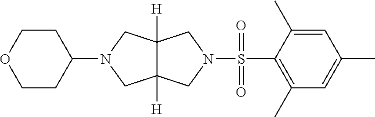 [SMILES: Cc1cc(C)c(S(=O)(=O)N2CC3CN(C4CCOCC4)CC3C2)c(C)c1]

2-(Mesitylsulfonyl)-5-(tetrahydro-2H-pyran-4-yl)octahydropyrrolo[3,4-c]pyrrole was obtained (20 mg, 37%) from tert-butyl-hexahydropyrrolo[3,4-c]pyrrole-2(1H)-carboxylate, 2,4,6-trimethylbenzenesulfonyl chloride and tetrahydro-4H-pyran-4-one, following a similar reaction sequence to those described in Example 31, steps 1-3. LCMS m/z=379.2 [M+H]⁺. ¹H-NMR (400 MHz, MeOH-d₄): δ (ppm) 7.04-6.90 (m, 2H), 3.92-3.74 (m, 2H), 3.29 (td, J=11.9, 2.1 Hz, 2H), 3.15-3.07 (m, 2H), 3.00-2.91 (m, 4H), 2.80-2.67 (m, 2H), 2.53 (s, 6H), 2.21 (s, 3H), 2.19-2.12 (m, 1H), 2.09 (dd, J=9.4, 5.6 Hz, 2H), 1.77-1.66 (m, 2H), 1.39 (qd, J=12.0, 4.5 Hz, 2H).

### Example 33: 3-Fluoro-5-(((3aR,6aS)-5-(tetrahydro-2H-pyran-4-yl)hexahydropyrrolo[3,4-c]pyrrol-2(1H)-yl)sulfonyl)benzonitrile

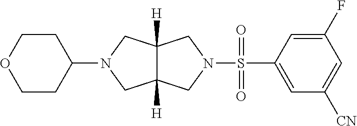 [SMILES: N#Cc1cc(F)cc(S(=O)(=O)N2C[C@H]3CN(C4CCOCC4)C[C@H]3C2)c1]

### 1. Synthesis of tert-butyl (3aR,6aS)-5-((3-cyano-5-fluorophenyl)sulfonyl)hexahydropyrrolo[3,4-c]pyrrole-2(1H)-carboxylate

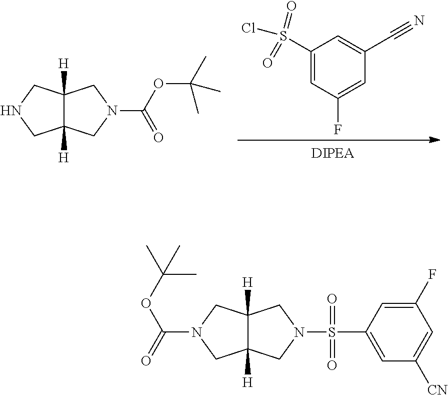 [SMILES: CC(C)(C)OC(=O)N1C[C@H]2CNC[C@H]2C1 ; N#Cc1cc(F)cc(S(=O)(=O)Cl)c1 ; CC(C)(C)OC(=O)N1C[C@@H]2CN(S(=O)(=O)c3cc(F)cc(C#N)c3)C[C@@H]2C1]

To a vial containing tert-butyl (3aR,6aS)-2,3,3a,4,6,6a-hexahydro-1H-pyrrolo[3,4-c]pyrrole-5-carboxylate (431 mg, 2.0 mmol) in anhydrous DCM (10 mL) was added DIPEA (1.3 mL, 7.5 mmol) dropwise at <5° C. After 5 mins, 3-cyano-5-fluorobenzenesulfonyl chloride (540 mg, 2.5 mmol) was added carefully to the cold solution. Upon complete addition, the reaction was warmed to 23° C. and stirred for 30 mins. The reaction was quenched by slow addition of aqueous 1 M sodium hydroxide solution. The mixture was stirred at 23° C. for 20 mins, then the biphasic mixture was extracted three times with DCM. The combined organic extracts were dried over anhydrous magnesium sulfate. After filtration and concentration under reduced pressure, the residue was loaded onto a silica gel column and purified with (10-75% EtOAc in heptane) to give tert-butyl (3aR,6aS)-5-((3-cyano-5-fluorophenyl)sulfonyl)hexahydropyrrolo[3,4-c]pyrrole-2(1H)-carboxylate (740 mg, 92%) as a white solid. ¹H-NMR (400 MHz, DMSO-d₆) δ (ppm) 8.32-8.28 (m, 1H), 8.16 (t, J=1.4 Hz, 1H), 8.06-8.02 (m, 1H), 3.47-3.36 (m, 3H), 3.16-2.68 (m, 7H), 1.35 (s, 9H).

### 2. Synthesis of 3-fluoro-5-(((3aR,6aS)-hexahydropyrrolo[3,4-c]pyrrol-2(1H)-yl)sulfonyl)benzonitrile

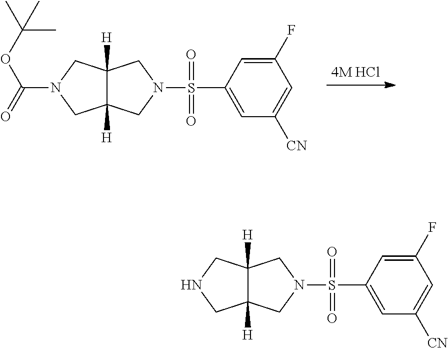 [SMILES: CC(C)(C)OC(=O)N1C[C@@H]2CN(S(=O)(=O)c3cc(F)cc(C#N)c3)C[C@@H]2C1 ; N#Cc1cc(F)cc(S(=O)(=O)N2C[C@H]3CNC[C@H]3C2)c1]

To a vial containing tert-butyl (3aR,6aS)-5-((3-cyano-5-fluorophenyl)sulfonyl)hexahydropyrrolo[3,4-c]pyrrole-2(1H)-carboxylate (292 mg, 0.7 mmol) in anhydrous MeOH (4 mL) was added 4 M HCl in dioxane (0.55 mL) dropwise at 23° C. Upon complete addition the reaction was stirred at rt for 21 h. The mixture was filtered to afford 3-fluoro-5-(((3aR,6aS)-hexahydropyrrolo[3,4-c]pyrrol-2(1H)-yl)sulfonyl)benzonitrile (HCl salt) (180 mg) as a white solid, that was used without purification.

LCMS m/z=296.1 [M+H]⁺.

### 3. Synthesis of 3-fluoro-5-(((3aR,6aS)-5-(tetrahydro-2H-pyran-4-yl)hexahydropyrrolo[3,4-c]pyrrol-2(1H)-yl)sulfonyl)benzonitrile

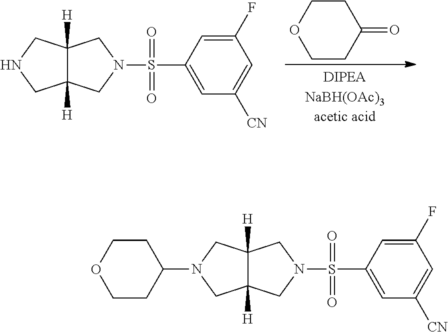 [SMILES: N#Cc1cc(F)cc(S(=O)(=O)N2C[C@H]3CNC[C@H]3C2)c1 ; O=C1CCOCC1 ; N#Cc1cc(F)cc(S(=O)(=O)N2C[C@H]3CN(C4CCOCC4)C[C@H]3C2)c1]

A vial containing 3-fluoro-5-(((3aR,6aS)-hexahydropyrrolo[3,4-c]pyrrol-2(1H)-yl)sulfonyl)benzonitrile (102 mg, 0.3 mmol) as HCl salt in anhydrous MeOH (3 mL) was cooled in an ice water bath, then DIPEA (0.2 mL, 1.1 mmol) was added. After 20 mins, tetrahydro-4H-pyran-4-one (0.06 mL, 0.65 mmol) and acetic acid (0.1 mL, 1.7 mmol) were added to the cooled mixture. After 15 mins, sodium triacetoxyborohydride (326 mg, 1.5 mmol) was added in portions to the cooled solution and the reaction stirred at <5° C. for 2 h. The reaction was quenched by slow addition of aqueous saturated NaHCO₃ solution. The mixture was stirred at 23° C. for 30 mins, then extracted three times with DCM. The combined organic extracts were dried over anhydrous sodium sulfate, filtered and concentrated under reduced pressure. The residue was loaded onto a silica gel column and purified with (40-100% 3:1 EtOAc/EtOH in heptane) to afford 3-fluoro-5-(((3aR,6aS)-5-(tetrahydro-2H-pyran-4-yl)hexahydropyrrolo[3,4-c]pyrrol-2(1H)-yl)sulfonyl)benzonitrile as a white solid (62 mg, 50%). LCMS m/z=380.2 [M+H]⁺. ¹H-NMR (400 MHz, DCM-d₂) δ (ppm) 7.91 (t, J=1.5 Hz, 1H), 7.78-7.75 (m, 1H), 7.63 (ddd, J=1.5, 2.5, 7.8 Hz, 1H), 3.85 (br t, J=3.5 Hz, 1H), 3.83-3.81 (m, 1H), 3.35-3.30 (m, 4H), 2.97-2.94 (m, 2H), 2.77-2.73 (m, 2H), 2.60-2.56 (m, 2H), 2.42-2.39 (m, 2H), 2.19-2.14 (m, 1H), 1.70-1.65 (m, 2H), 1.43-1.36 (m, 2H).

### Example 34: (3aR,6aS)-2-((6-Methoxy-2-methylpyridin-3-yl)sulfonyl)-5-(tetrahydro-2H-pyran-4-yl)octahydropyrrolo[3,4-c]pyrrole

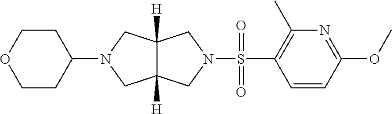 [SMILES: COc1ccc(S(=O)(=O)N2C[C@H]3CN(C4CCOCC4)C[C@H]3C2)c(C)n1]

### 1. Synthesis of tert-butyl (3aR,6aS)-5-(tetrahydro-2H-pyran-4-yl)hexahydropyrrolo[3,4-c]pyrrole-2(1H)-carboxylate

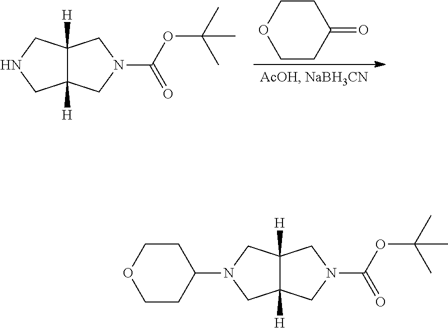

To a solution of tetrahydro-4H-pyran-4-one (637 mg, 6.4 mmol) and tert-butyl (3aR,6aS)-hexahydropyrrolo[3,4-c]pyrrole-2(1H)-carboxylate (900 mg, 4.2 mmol) in MeOH (10 mL) was added acetic acid (0.1 mL) and sodium cyanoborohydride (1.1 g, 17.0 mmol) at rt and the reaction heated at 30° C. for 1 h. The mixture was quenched with saturated. aq. NaHCO₃ (until pH=8), diluted with water (150 mL) and extracted with DCM (3×90 mL). The combined organic layers were dried over Na₂SO₄, filtered and concentrated to give the residue, which was purified by column chromatography (0% to 10% MeOH in DCM) to give tert-butyl (3aR,6aS)-5-(tetrahydro-2H-pyran-4-yl)hexahydropyrrolo[3,4-c]pyrrole-2(1H)-carboxylate (1.1 g, 88%) as a light yellow solid. ¹H-NMR (400 MHz, CDCl₃) δ (ppm): 4.09-3.94 (m, 2H), 3.59-3.45 (m, 2H), 3.45-3.36 (m, 2H), 3.34-3.22 (m, 2H), 3.11-2.81 (m, 3H), 2.50-2.28 (m, 2H), 1.89-1.77 (m, 2H), 1.71-1.57 (m, 4H), 1.48 (s, 9H).

### 2. Synthesis of (3aR,6aS)-2-(tetrahydro-2H-pyran-4-yl)octahydropyrrolo[3,4-c]pyrrole

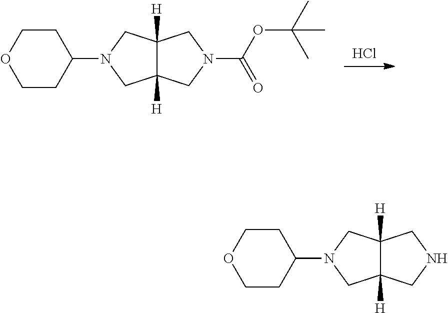 [SMILES: CC(C)(C)OC(=O)N1C[C@@H]2CN(C3CCOCC3)C[C@@H]2C1 ; Cl ; C1CC(N2C[C@H]3CNC[C@H]3C2)CCO1]

A solution of tert-butyl (3aR,6aS)-5-(tetrahydro-2H-pyran-4-yl)hexahydropyrrolo[3,4-c]pyrrole-2(1H)-carboxylate (500 mg, 1.7 mmol) in 1N HCl/MeOH (30 mL) was stirred at 19° C. for 2 h. The mixture was concentrated under reduced pressure to give (3aR,6aS)-2-(tetrahydro-2H-pyran-4-yl)octahydropyrrolo[3,4-c]pyrrole (450 mg, as HCl salt) as a brown solid which was used without further purification. ¹H-NMR (400 MHz, MeOH-d₄) δ (ppm): 4.10-3.93 (m, 3H), 3.86-3.78 (m, 1H), 3.62 (s, 2H), 3.54-3.49 (m, 2H), 3.49-3.34 (m, 7H), 2.08 (br d, J=12.4 Hz, 3H), 1.84-1.68 (m, 1H).

### 3. Synthesis of (3aR,6aS)-2-((6-methoxy-2-methylpyridin-3-yl)sulfonyl)-5-(tetrahydro-2H-pyran-4-yl)octahydropyrrolo[3,4-c]pyrrole

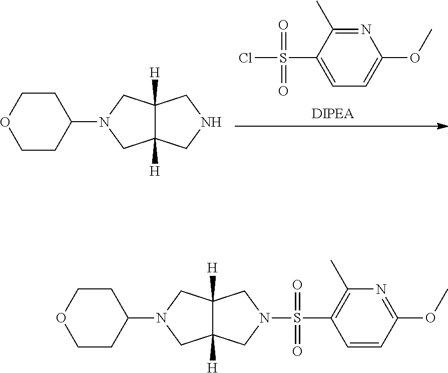 [SMILES: C1CC(N2C[C@H]3CNC[C@H]3C2)CCO1 ; COc1ccc(S(=O)(=O)Cl)c(C)n1 ; COc1ccc(S(=O)(=O)N2C[C@H]3CN(C4CCOCC4)C[C@H]3C2)c(C)n1]

To a solution of (3aR,6aS)-2-(tetrahydro-2H-pyran-4-yl)octahydropyrrolo[3,4-c]pyrrole (90 mg, 0.4 mmol) as HCl salt and 6-methoxy-2-methyl-pyridine-3-sulfonyl chloride (86 mg, 0.4 mmol) in DCM (2 mL) was added DIPEA (270 μL, 1.6 mmol) at 20° C. After 2 h the mixture was concentrated under reduced pressure and the resulting residue was purified by column chromatography (MeOH in DCM 0% to 10%). The product was further purified by prep-HPLC (Column: Waters Xbridge 150*25 mm 10 μm; Condition: (A) water (NH₄HCO₃)—(B) MeCN; Begin B: 25; End B: 55; Gradient Time (min): 8; FlowRate (mL/min): 30) to afford ((3aR,6aS)-2-((6-methoxy-2-methylpyridin-3-yl)sulfonyl)-5-(tetrahydro-2H-pyran-4-yl)octahydropyrrolo[3,4-c]pyrrole (61 mg, 41%) as a white solid. LCMS m/z=382.1 [M+H]⁺. ¹H-NMR (400 MHz, MeOH-d₄) δ (ppm): 8.07 (d, J=8.8 Hz, 1H), 6.75 (d, J=8.8 Hz, 1H), 3.98 (s, 3H), 3.95-3.89 (m, 2H), 3.44-3.37 (m, 2H), 3.21-3.15 (m, 2H), 3.11-3.05 (m, 2H), 3.00-2.93 (m, 2H), 2.85-2.79 (m, 2H), 2.76 (s, 3H), 2.28-2.20 (m, 3H), 1.86-1.77 (m, 2H), 1.53-1.43 (m, 2H).

### Example 35: (3aR,6aS)-2-((2-Methyl-6-(trifluoromethyl)pyridin-3-yl)sulfonyl)-5-(tetrahydro-2H-pyran-4-yl)octahydropyrrolo[3,4-c]pyrrole

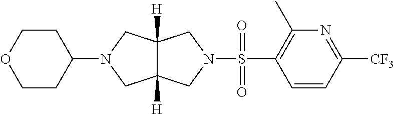 [SMILES: Cc1nc(C(F)(F)F)ccc1S(=O)(=O)N1C[C@H]2CN(C3CCOCC3)C[C@H]2C1]

(3aR,6aS)-2-((2-Methyl-6-(trifluoromethyl)pyridin-3-yl)sulfonyl)-5-(tetrahydro-2H-pyran-4-yl)octahydropyrrolo[3,4-c]pyrrole was obtained as a white solid (72 mg, 44%) from 2-methyl-6-(trifluoromethyl)pyridine-3-sulfonyl chloride and (3aR,6aS)-2-(tetrahydro-2H-pyran-4-yl)octahydropyrrolo[3,4-c]pyrrole as HCl salt (Example 34, step 2), following a similar method to that described in Example 34, step 3, except the HPLC gradient used was (A) water (NH₄HCO₃)—(B) MeCN; Begin B: 31; End B: 61; Gradient Time (min): 8; FlowRate (mL/min): 30). LCMS m/z: 420.0 [M+H]⁺. ¹H-NMR (400 MHz, MeOH-d₄) δ (ppm): 8.40 (d, J=8.0 Hz, 1H), 7.66 (d, J=8.0 Hz, 1H), 4.03-3.89 (m, 2H), 3.44-3.32 (m, 4H), 3.23-3.09 (m, 2H), 3.01-2.92 (m, 3H), 2.90-2.75 (m, 4H), 2.45-2.31 (m, 2H), 2.28-2.17 (m, 1H), 1.78-1.68 (m, 2H), 1.61-1.46 (m, 2H).

### Example 36: (3aR,6aS)-2-((1-Methyl-3-(trifluoromethyl)-1H-pyrazol-5-yl)sulfonyl)-5-(tetrahydro-2H-pyran-4-yl)octahydropyrrolo[3,4-c]pyrrole

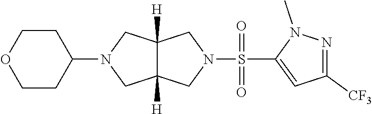 [SMILES: Cn1nc(C(F)(F)F)cc1S(=O)(=O)N1C[C@H]2CN(C3CCOCC3)C[C@H]2C1]

(3aR,6aS)-2-((1-Methyl-3-(trifluoromethyl)-1H-pyrazol-5-yl)sulfonyl)-5-(tetrahydro-2H-pyran-4-yl)octahydropyrrolo[3,4-c]pyrrole was obtained as a white solid (32 mg, 20%) from 2-methyl-5-(trifluoromethyl)pyrazole-3-sulfonyl chloride and (3aR,6aS)-2-(tetrahydro-2H-pyran-4-yl)octahydropyrrolo[3,4-c]pyrrole as HCl salt (Example 34, step 2), following a similar method to that described in Example 34, step 3, except the crude material was purified by prep-HPLC (Column: Welch Xtimate C18 150*25 mm*5 μm; Condition: (A) water (NH₃H₂O)—(B) MeCN; Begin B: 32; End B: 62; Gradient Time (min): 8; FlowRate (mL/min); 30) (twice). LCMS m/z=409.1 [M+H]⁺. ¹H-NMR (400 MHz, CDCl₃) δ (ppm): 7.00 (s, 1H), 4.17 (s, 3H), 4.00-3.93 (m, 2H), 3.42-3.34 (m, 4H), 3.28-3.18 (m, 2H), 3.02-2.73 (m, 4H), 2.51-2.24 (m, 3H), 1.80-1.72 (m, 2H), 1.63-1.50 (m, 2H).

### Example 37: (3aR,6aS)-2-((4-Methyl-2-(trifluoromethyl)pyrimidin-5-yl)sulfonyl)-5-(tetrahydro-2H-pyran-4-yl)octahydropyrrolo[3,4-c]pyrrole

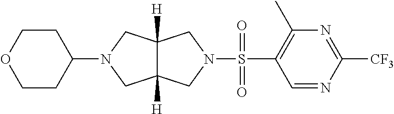 [SMILES: Cc1nc(C(F)(F)F)ncc1S(=O)(=O)N1C[C@H]2CN(C3CCOCC3)C[C@H]2C1]

(3aR,6aS)-2-((4-Methyl-2-(trifluoromethyl)pyrimidin-5-yl)sulfonyl)-5-(tetrahydro-2H-pyran-4-yl)octahydropyrrolo[3,4-c]pyrrole was obtained as a white solid, 6 mg, 4% from 4-methyl-2-(trifluoromethyl)pyrimidine-5-sulfonyl chloride and (3aR,6aS)-2-(tetrahydro-2H-pyran-4-yl)octahydropyrrolo[3,4-c]pyrrole as HCl salt (Example 34, step 2), following a similar procedure to that described in Example 34, step 3, except, the crude material was purified by prep-HPLC (Column: Welch Xtimate C18 150*25 mm*5 μm, Condition: (A) water (NH₄HCO₃)—(B) MeCN, 30%˜60%, Flow Rate (mL/min): 25). LCMS m/z=421.1 [M+H]⁺. ¹H-NMR (400 MHz, CDCl₃) δ (ppm): 9.24 (s, 1H), 3.96 (d, J=10.0 Hz, 2H), 3.54-3.47 (m, 2H), 3.41-3.34 (m, 2H), 3.20 (d, J=10.4 Hz, 2H), 2.97 (s, 3H), 2.89 (s, 2H), 2.72 (s, 2H), 2.45 (d, J=6.8 Hz, 2H), 2.22 (s, 1H), 1.73 (d, J=12.0 Hz, 2H), 1.51 (br s, 2H).

### Example 38: (3aR,6aS)-2-((2-Chloro-6-methoxypyridin-3-yl)sulfonyl)-5-(tetrahydro-2H-pyran-4-yl)octahydropyrrolo[3,4-c]pyrrole

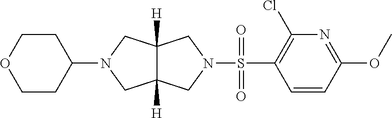 [SMILES: COc1ccc(S(=O)(=O)N2C[C@H]3CN(C4CCOCC4)C[C@H]3C2)c(Cl)n1]

(3aR,6aS)-2-((2-Chloro-6-methoxypyridin-3-yl)sulfonyl)-5-(tetrahydro-2H-pyran-4-yl)octahydropyrrolo[3,4-c]pyrrole was obtained as a white solid, from 2-chloro-6-methoxy-pyridine-3-sulfonyl chloride and (3aR,6aS)-2-(tetrahydro-2H-pyran-4-yl)octahydropyrrolo[3,4-c]pyrrole as HCl salt (Example 34, step 2), following a similar procedure to that described in Example 34, step 3, except the crude material was purified by prep-HPLC (Column: Welch Xtimate: C18 150*25 mm*5 μm; Condition: (A) water (NH₄HCO₃)—(B) MeCN; Begin B: 24%; End B: 54%; FlowRate (mL/min): 25). LCMS m/z=402.1 [M+H]⁺. ¹H-NMR (400 MHz, MeOH-d₄) δ (ppm): 8.24 (d, J=12.0 Hz, 1H), 6.89 (d, J=8.0 Hz, 1H), 4.00 (s, 3H), 3.92 (br dd, J=4.0, 12.0 Hz, 2H), 3.45-3.32 (m, 4H), 3.22 (dd, J=8.0, 12.0 Hz, 2H), 3.02-2.93 (m, 2H), 2.88-2.76 (m, 2H), 2.36-2.12 (m, 3H), 1.82 (br dd, J=4.0, 12.0 Hz, 2H), 1.54-1.42 (m, 2H).

### Example 39: (3aR,6aS)-2-((3,5-Difluorophenyl)sulfonyl)-5-(tetrahydro-2H-pyran-4-yl)octahydropyrrolo[3,4-c]pyrrole

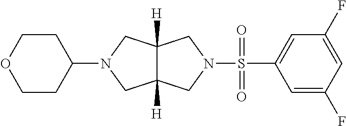 [SMILES: O=S(=O)(c1cc(F)cc(F)c1)N1C[C@H]2CN(C3CCOCC3)C[C@H]2C1]

(3aR,6aS)-2-((3,5-Difluorophenyl)sulfonyl)-5-(tetrahydro-2H-pyran-4-yl)octahydropyrrolo[3,4-c]pyrrole was obtained as a white solid (65 mg, 34%) from 3,5-difluorobenzenesulfonyl chloride and (3aR,6aS)-2-(tetrahydro-2H-pyran-4-yl)octahydropyrrolo[3,4-c]pyrrole as HCl salt (Example 34, step 2), following a similar procedure to that described in Example 34, step 3, except the crude material was purified by prep-HPLC (Boston Prime C18 150*30 mm*5 μm, Condition: (A) water (NH₃H₂O+NH₄HCO₃)—(B) MeCN, Flow Rate (mL/min): 25). LCMS m/z=373.1 [M+H]⁺. ¹H-NMR (400 MHz, CDCl₃) δ (ppm): 7.37-7.32 (m, 2H), 7.10-7.05 (m, 1H), 3.94-3.91 (m, 2H), 3.40-3.34 (m, 2H), 3.16-3.06 (m, 4H), 2.88-2.78 (m, 4H), 2.29-2.19 (m, 3H), 1.75-1.72 (m, 2H), 1.54-1.44 (m, 2H).

### Example 40: (3aR,6aS)-2-((6-Methoxypyridin-3-yl)sulfonyl)-5-(tetrahydro-2H-pyran-4-yl)octahydropyrrolo[3,4-c]pyrrole

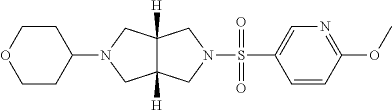 [SMILES: COc1ccc(S(=O)(=O)N2C[C@H]3CN(C4CCOCC4)C[C@H]3C2)cn1]

(3aR,6aS)-2-((6-Methoxypyridin-3-yl)sulfonyl)-5-(tetrahydro-2H-pyran-4-yl)octahydropyrrolo[3,4-c]pyrrole was obtained as a white solid, 22 mg, 17% from 6-methoxypyridine-3-sulfonyl chloride and (3aR,6aS)-2-(tetrahydro-2H-pyran-4-yl)octahydropyrrolo[3,4-c]pyrrole as HCl salt (Example 34, step 2), following a similar procedure to that described in Example 34, step 3, except, the crude material was purified by prep-HPLC (Welch Xtimate C18 150*25 mm*5 μm, Condition: water (NH₄HCO₃)-MeCN, Flow Rate (mL/min): 25). LCMS m/z=368.1 [M+H]⁺. ¹H-NMR (400 MHz, CDCl₃) δ (ppm): 8.61 (d, J=2.4 Hz, 1H), 7.92 (dd, J=2.4, 8.8 Hz, 1H), 6.65 (d, J=8.8 Hz, 1H), 4.02 (s, 3H), 3.96-3.93 (m, 2H), 3.40-3.34 (m, 2H), 3.13-3.11 (m, 2H), 3.03 (m, 3H), 2.83-2.82 (m, 2H), 2.36-2.31 (m, 2H), 1.78-1.75 (m, 2H), 1.66-1.58 (m, 4H).

### Example 41: (3aR,6aS)-2-((5-Chloro-2-methoxypyridin-3-yl)sulfonyl)-5-(tetrahydro-2H-pyran-4-yl)octahydropyrrolo[3,4-c]pyrrole

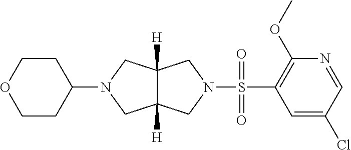 [SMILES: COc1ncc(Cl)cc1S(=O)(=O)N1C[C@H]2CN(C3CCOCC3)C[C@H]2C1]

(3aR,6aS)-2-((5-Chloro-2-methoxypyridin-3-yl)sulfonyl)-5-(tetrahydro-2H-pyran-4-yl)octahydropyrrolo[3,4-c]pyrrole was obtained as a white solid (50 mg, 27%) from 5-chloro-2-methoxy-pyridine-3-sulfonyl chloride and (3aR,6aS)-2-(tetrahydro-2H-pyran-4-yl)octahydropyrrolo[3,4-c]pyrrole as HCl salt (Example 34, step 2), following a similar procedure to that described in Example 34, step 3, except, the crude material was purified by prep-HPLC (Welch Xtimate C18 150*25 mm*5 μm, Condition: water (NH₄HCO₃)-MeCN, Flow Rate (mL/min): 25). LCMS m/z=402.1 [M+H]⁺. ¹H-NMR (400 MHz, CDCl₃) δ (ppm): 8.26 (d, J=2.4 Hz, 1H), 8.16 (d, J=2.8 Hz, 1H), 4.05 (s, 3H), 3.97-3.94 (m, 2H), 3.47-3.42 (m, 2H), 3.41-3.35 (m, 2H), 3.28-3.26 (m, 2H), 2.88-2.81 (m, 4H), 2.32-2.31 (m, 2H), 2.28-2.24 (m, 1H), 1.77-1.74 (m, 2H), 1.54-1.52 (m, 2H).

### Example 42: (3aR,6aS)-2-((2-Methoxy-5-methylpyridin-3-yl)sulfonyl)-5-(tetrahydro-2H-pyran-4-yl)octahydropyrrolo[3,4-c]pyrrole

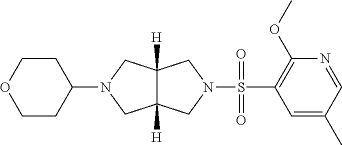 [SMILES: COc1ncc(C)cc1S(=O)(=O)N1C[C@H]2CN(C3CCOCC3)C[C@H]2C1]

(3aR,6aS)-2-((2-Methoxy-5-methylpyridin-3-yl)sulfonyl)-5-(tetrahydro-2H-pyran-4-yl)octahydropyrrolo[3,4-c]pyrrole was obtained as a white solid (61 mg, 35%) from 2-methoxy-5-methyl-pyridine-3-sulfonyl chloride and (3aR,6aS)-2-(tetrahydro-2H-pyran-4-yl)octahydropyrrolo[3,4-c]pyrrole as HCl salt (Example 34, step 2), following a similar procedure to that described in Example 34, step 3, except the crude material was purified by prep-HPLC (Welch Xtimate C18 150*25 mm*5 μm, Condition: water (NH₄HCO₃)-MeCN, Flow Rate (mL/min): 25). LCMS m/z=382.1 [M+H]⁺. ¹H-NMR (400 MHz, CDCl₃) δ (ppm): 8.12-8.11 (m, 1H), 8.00-7.99 (m, 1H), 4.02 (s, 3H), 3.97-3.94 (m, 2H), 3.41-3.35 (m, 4H), 3.26-3.23 (m, 2H), 2.92 (m, 2H), 2.78 (m, 2H), 2.31 (s, 3H), 2.29-2.23 (m, 3H), 1.77-1.74 (m, 2H), 1.61-1.47 (m, 2H).

### Example 43: (3aR,6aS)-2-((2,4-Dimethylpyrimidin-5-yl)sulfonyl)-5-(tetrahydro-2H-pyran-4-yl)octahydropyrrolo[3,4-c]pyrrole

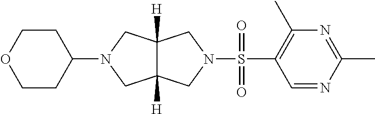 [SMILES: Cc1ncc(S(=O)(=O)N2C[C@H]3CN(C4CCOCC4)C[C@H]3C2)c(C)n1]

(3aR,6aS)-2-((2,4-Dimethylpyrimidin-5-yl)sulfonyl)-5-(tetrahydro-2H-pyran-4-yl)octahydropyrrolo[3,4-c]pyrrole was obtained as a white solid (55 mg, 41%) from 2,4-dimethylpyrimidine-5-sulfonyl chloride and (3aR,6aS)-2-(tetrahydro-2H-pyran-4-yl)octahydropyrrolo[3,4-c]pyrrole as HCl salt (Example 34, step 2), following a similar procedure to that described in Example 34, step 3, except the crude material was purified by prep-HPLC (Column: Welch Xtimate C18 150*25 mm*5 μm; Condition: (A) water (NH₄HCO₃)—(B) MeCN; Begin B:15; End B: 45; Flow Rate (mL/min): 25). LCMS m/z=367.1 [M+H]⁺. ¹H-NMR (400 MHz, CDCl₃) δ (ppm): 8.98 (s, 1H), 3.95 (br d, J=10.4 Hz, 2H), 3.42-3.34 (m, 4H), 3.15-3.13 (m, 2H), 2.89-2.83 (m, 2H), 2.83 (s, 3H), 2.82-2.78 (m, 2H), 2.78 (s, 3H), 2.34 (br d, J=5.6 Hz, 2H), 2.25-2.15 (m, 1H), 1.73 (br d, J=12.0 Hz, 2H), 1.54-1.44 (m, 2H).

### Example 44: (3aR,6aS)-2-((2,4-Dimethylpyrimidin-5-yl)sulfonyl)-5-(tetrahydrofuran-3-yl)octahydropyrrolo[3,4-c]pyrrole

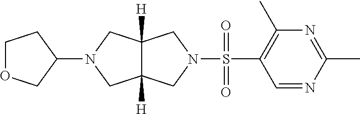 [SMILES: Cc1ncc(S(=O)(=O)N2C[C@H]3CN(C4CCOC4)C[C@H]3C2)c(C)n1]

(3aR,6aS)-2-((2,4-Dimethylpyrimidin-5-yl)sulfonyl)-5-(tetrahydrofuran-3-yl)octahydropyrrolo[3,4-c]pyrrole was obtained as a white solid (58 mg, 18%) from tert-butyl (3aR,6aS)-hexahydropyrrolo[3,4-c]pyrrole-2(1H)-carboxylate and 2,4-dimethylpyrimidine-5-sulfonyl chloride in step 1 and tetrahydrofuran-3-one in step 3, following a similar reaction sequence to that described in Example 31. The crude material was purified by prep-HPLC (Column: Waters Xbridge BEH C18 100*30 mm*10 μm; Condition: (A) water (NH₄HCO₃)-(B) MeCN; Begin B:10; End B: 40; Flow Rate (mL/min): 50). LCMS m/z=353.1 [M+H]⁺. ¹H-NMR (400 MHz, CDCl₃) δ (ppm): 8.98 (s, 1H), 3.94-3.86 (m, 1H), 3.84-3.75 (m, 2H), 3.58-3.50 (m, 1H), 3.43-3.34 (m, 2H), 3.14-3.10 (m, 2H), 2.85 (s, 3H), 2.82 (s, 3H), 2.78 (s, 3H), 2.76-2.63 (m, 2H), 2.40-2.26 (m, 2H), 2.06-1.94 (m, 1H), 1.88-1.76 (m, 1H).

### Example 45: (3aR,6aR)-2-((2-Methyl-6-(trifluoromethyl)pyridin-3-yl)sulfonyl)-5-(tetrahydro-2H-pyran-4-yl)octahydropyrrolo[3,4-c]pyrrole

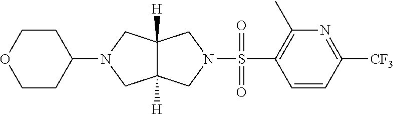 [SMILES: Cc1nc(C(F)(F)F)ccc1S(=O)(=O)N1C[C@H]2CN(C3CCOCC3)C[C@@H]2C1]

### 1. Synthesis of tert-butyl (3aR,6aR)-5-(tetrahydro-2H-pyran-4-yl)hexahydropyrrolo[3,4-c]pyrrole-2(1H)-carboxylate

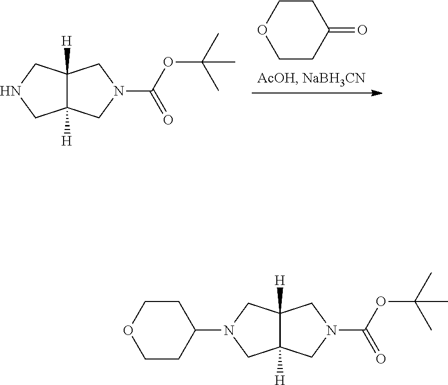

To a solution of tetrahydro-4H-pyran-4-one (177 mg, 1.8 mmol) and tert-butyl (3aR,6aR)-hexahydropyrrolo[3,4-c]pyrrole-2(1H)-carboxylate (250 mg, 1.2 mmol) in MeOH (10 mL) was added acetic acid (0.1 mL) and sodium cyanoborohydride (296 mg, 4.7 mmol) at 16° C., and the reaction was stirred at 30° C. for 1 h. The mixture was quenched with sat. NaHCO₃ (until pH=8), diluted with water (150 mL) and extracted with DCM (90 mL×3). The combined organic layers were dried over Na₂SO₄, filtered and concentrated. The residue was purified by Combiflash® (MeOH in DCM from 0% to 10%) to give tert-butyl (3aR,6aR)-5-(tetrahydro-2H-pyran-4-yl)hexahydropyrrolo[3,4-c]pyrrole-2(1H)-carboxylate (300 mg, 86%) as a pale yellow oil. ¹H-NMR (400 MHz, CDCl₃) δ (ppm): 3.97-3.95 (m, 2H), 3.88-3.85 (m, 2H), 3.67-3.64 (m, 2H), 3.45-3.44 (m, 2H), 3.43-3.38 (m, 3H), 1.91-1.90 (m, 2H), 1.61-1.59 (m, 2H), 1.57-1.55 (m, 4H), 1.46 (s, 9H).

### 2. Synthesis of (3aS,6aS)-2-(tetrahydro-2H-pyran-4-yl)octahydropyrrolo[3,4-c]pyrrole

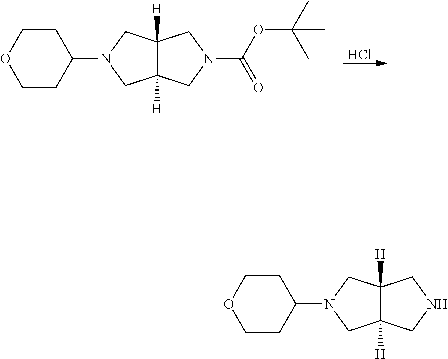 [SMILES: CC(C)(C)OC(=O)N1C[C@H]2CN(C3CCOCC3)C[C@@H]2C1 ; Cl ; C1CC(N2C[C@@H]3CNC[C@H]3C2)CCO1]

A solution of tert-butyl (3aR,6aR)-5-(tetrahydro-2H-pyran-4-yl)hexahydropropyrrolo[3,4-c]pyrrole-2(1H)-carboxylate (100 mg, 0.337 mmol) in HCl/MeOH (30 mL) was stirred at 19° C. for 12 h. The mixture was concentrated to give (3aS,6aS)-2-(tetrahydro-2H-pyran-4-yl)octahydropyrrolo[3,4-c]pyrrole (80 mg, crude, as the HCl salt) as a brown solid. ¹H-NMR: (400 MHz, MeOH-d₄) δ ppm 4.17-3.99 (m, 2H), 3.97-3.87 (m, 1H), 3.84-3.70 (m, 2H), 3.62-3.55 (m, 2H), 3.48-3.43 (m, 2H), 3.22-3.11 (m, 2H), 2.89-2.57 (m, 2H), 2.17-1.98 (m, 2H), 1.93-1.68 (m, 3H), 1.59-1.49 (m, 1H)

### 3. Synthesis of (3aR,6aR)-2-((2-methyl-6-(trifluoromethyl)pyridin-3-yl)sulfonyl)-5-(tetrahydro-2H-pyran-4-yl)octahydropyrrolo[3,4-c]pyrrole

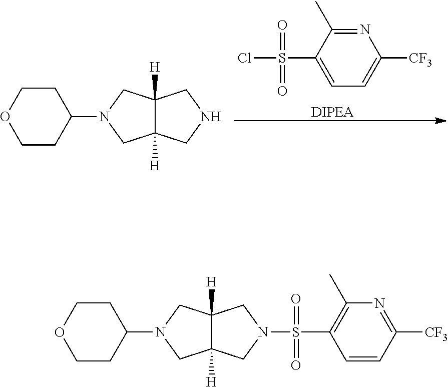

To a solution of (3aS,6aS)-2-(tetrahydro-2H-pyran-4-yl)octahydropyrrolo[3,4-c]pyrrole (72 mg, 0.3 mmol, HCl salt) and 2-methyl-6-(trifluoromethyl)pyridine-3-sulfonyl chloride (80 mg, 0.3 mmol) in DCM (5 mL) was added DIPEA (159 mg, 1.2 mmol) and the reaction was stirred at 20° C. for 2 h. The mixture was concentrated to give the residue, which was purified by prep-HPLC (Column: Waters xbridge 150*25 mm 10 m; Condition: (A) water (NH₄HCO₃)—(B) MeCN; Begin B: 31; End B: 61; Gradient Time (min): 8; Flow Rate (mL/min): 30) to give (3aR,6aR)-2-((2-methyl-6-(trifluoromethyl)pyridin-3-yl)sulfonyl)-5-(tetrahydro-2H-pyran-4-yl)octahydropyrrolo[3,4-c]pyrrole (59 mg, 42%) as light yellow solid LCMS m/z=420.0 [M+H]⁺. ¹H-NMR (400 MHz, MeOH-d₄) δ (ppm): 8.43 (d, J=8.1 Hz, 1H), 7.82 (d, J=8.1 Hz, 1H), 3.99-3.89 (m, 2H), 3.65 (dd, J=6.3, 8.6 Hz, 2H), 3.46-3.34 (m, 2H), 3.09 (dd, J=8.8, 10.8 Hz, 2H), 3.01-2.93 (m, 2H), 2.90 (s, 3H), 2.74 (tt, J=3.9, 11.1 Hz, 1H), 2.59 (dd, J=9.3, 10.6 Hz, 2H), 2.35-2.21 (m, 2H), 1.92-1.73 (m, 2H), 1.60-1.38 (m, 2H).

### Example 46: (3aR,6aR)-2-((1-Methyl-3-(trifluoromethyl)-1H-pyrazol-5-yl)sulfonyl)-5-(tetrahydro-2H-pyran-4-yl)octahydropyrrolo[3,4-c]pyrrole

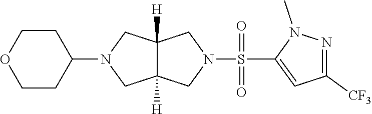 [SMILES: Cn1nc(C(F)(F)F)cc1S(=O)(=O)N1C[C@H]2CN(C3CCOCC3)C[C@@H]2C1]

To a solution of (3aS,6aS)-2-(tetrahydro-2H-pyran-4-yl)octahydropyrrolo[3,4-c]pyrrole (90 mg, 0.5 mmol, HCl salt) (Example 45, step 3) and 2-methyl-5-(trifluoromethyl)pyrazole-3-sulfonyl chloride (114 mg, 0.5 mmol) in DCM (4 mL) was added DIPEA (178 mg, 1.4 mmol) and the reaction was stirred at 20° C. for 2 h. The mixture was concentrated to give the residue, which was purified by prep-HPLC (Column: Phenomenex C18 150*25 mm*10 μm; Condition: (A) water (NH₄HCO₃)—(B) MeCN; Begin B: 24; End B: 54; Gradient Time (min): 8; Flow Rate (mL/min): 30) and further purified by prep-HPLC (Column: Phenomenex luna C18 150*25 mm*110 μm; Condition: (A) water (FA)-(B) MeCN; Begin B: 9; End B: 39; Gradient Time (min): 10; Flow Rate (mL/min): 25) to give (3aR,6aR)-2-((1-methyl-3-(trifluoromethyl)-1H-pyrazol-5-yl)sulfonyl)-5-(tetrahydro-2H-pyran-4-yl)octahydropyrrolo[3,4-c]pyrrole (43 mg, 20%, Formate salt) as an off-white solid. LCMS m/z=409.1 [M+H]⁺. ¹H-NMR (400 MHz, MeOH-d₄) δ (ppm): 8.47 (s, 1H), 7.17 (s, 1H), 4.18 (s, 3H), 4.07-3.96 (m, 2H), 3.79-3.69 (m, 2H), 3.49-3.36 (m, 4H), 3.29-3.17 (m, 2H), 3.10-2.99 (m, 2H), 2.51-2.35 (m, 2H), 2.03-1.88 (m, 2H), 1.72-1.58 (m, 2H).

### Examples 47 and 48: (3aS,6aS)-2-((2-methyl-6-(trifluoromethyl)pyridin-3-yl)sulfonyl)-5-(tetrahydro-2H-pyran-4-yl)octahydropyrrolo[3,4-c]pyrrole and (3aR,6aR)-2-((2-Methyl-6-(trifluoromethyl)pyridin-3-yl)sulfonyl)-5-(tetrahydro-2H-pyran-4-yl)octahydropyrrolo[3,4-c]pyrrole

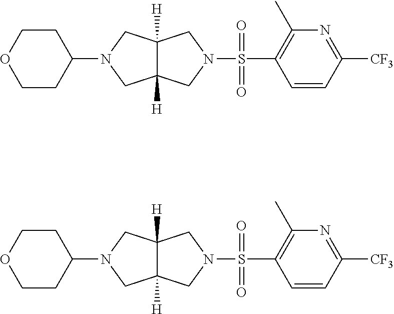 [SMILES: Cc1nc(C(F)(F)F)ccc1S(=O)(=O)N1C[C@@H]2CN(C3CCOCC3)C[C@H]2C1 ; Cc1nc(C(F)(F)F)ccc1S(=O)(=O)N1C[C@H]2CN(C3CCOCC3)C[C@@H]2C1]

Rac-(3aR,6aR)-2-((2-Methyl-6-(trifluoromethyl)pyridin-3-yl)sulfonyl)-5-(tetrahydro-2H-pyran-4-yl)octahydropyrrolo[3,4-c]pyrrole was resolved by chiral SFC purification using a (Column:DAICEL CHIRALPAK AD (250 mm*30 mm, 10 um); Mobile Phase: from 15% to 15% of Neu-ETOH; Flow Rate (mL/min): 150; Column temp: 35° C.) to give: (3aS,6aS)-2-((2-methyl-6-(trifluoromethyl)pyridin-3-yl)sulfonyl)-5-(tetrahydro-2H-pyran-4-yl)octahydropyrrolo[3,4-c]pyrrole (150 mg. 33.4%, Rf=2.024 min). LCMS m/z=420.1 [M+H−56]⁺. ¹HNMR (400 MHz, CDCl₃): δ ppm 8.35 (d, J=8.0 Hz, 1H), 7.65 (d, J=8.4 Hz, 1H), 4.00-3.97 (m, 2H), 3.67-3.63 (m, 2H), 3.41-3.35 (m, 2H), 3.10-3.05 (m, 2H), 3.02-2.98 (m, 2H), 2.92 (s, 3H), 2.78-2.71 (m, 1H), 2.65 (t, J=10.0 Hz, 2H), 2.35-2.26 (m, 2H), 1.82-1.72 (m, 2H), 1.62-1.50 (m, 2H). Compound chirality was assigned arbitrarily. (3aR,6aR)-2-((2-Methyl-6-(trifluoromethyl)pyridin-3-yl)sulfonyl)-5-(tetrahydro-2H-pyran-4-yl)octahydropyrrolo[3,4-c]pyrrole (120 mg. 26.7%, Rf=2.054 min). LCMS m/z=420.1 [M+H−56]⁺. ¹HNMR (400 MHz, CDCl₃): δ ppm 8.35 (d, J=8.0 Hz, 1H), 7.65 (d, J=8.4 Hz, 1H), 4.00-3.98 (m, 2H), 3.67-3.63 (m, 2H), 3.41-3.35 (m, 2H), 3.11-3.06 (m, 2H), 3.03-3.00 (m, 2H), 2.92 (s, 3H), 2.79-2.73 (m, 1H), 2.67 (t, J=9.6 Hz, 2H), 2.34-2.28 (m, 2H), 1.80-1.74 (m, 2H), 1.64-1.54 (m, 2H). Compound chirality was assigned arbitrarily.

### Example 49: Rac-(3aR,6aS)-2-((1,3-dimethyl-1H-pyrazol-5-yl)sulfonyl)-5-(tetrahydro-2H-pyran-4-yl)octahydropyrrolo[3,4-c]pyrrole

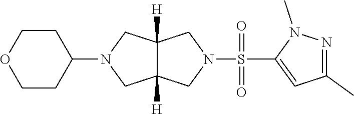 [SMILES: Cc1cc(S(=O)(=O)N2C[C@H]3CN(C4CCOCC4)C[C@H]3C2)n(C)n1]

Rac-(3aR,6aS)-2-((1,3-dimethyl-1H-pyrazol-5-yl)sulfonyl)-5-(tetrahydro-2H-pyran-4-yl)octahydropyrrolo[3,4-c]pyrrole was obtained (18 mg, yield 63%) as a white solid, from 1,3-dimethyl-1H-pyrazole-5-sulfonyl chloride and tert-butyl hexahydropyrrolo[3,4-c]pyrrole-2(1H)-carboxylate, following a similar procedure described in Example 33 (step 1-3), except the crude material was purified by normal phase column (4 g, EtOAc/EtOH 3/1 100%). LCMS m/z=355.2 [M+H]⁺. ¹HNMR (400 MHz, MeOH-d₄): δ (ppm) 6.50-6.67 (m, 1H), 4.02 (s, 3H), 3.87-3.97 (m, 2H), 3.40 (td, J=11.8, 2.0 Hz, 2H), 3.19-3.26 (m, 2H), 3.11-3.17 (m, 2H), 2.93 (br dd, J=9.5, 7.5 Hz, 2H), 2.82 (br d, J=3.5 Hz, 2H), 2.18-2.35 (m, 6H), 1.76-1.86 (m, 2H), 1.40-1.57 (m, 2H).

### Example 50: Rac-(3aR,6aR)-2-((2-methyl-6-(trifluoromethyl)pyridin-3-yl)sulfonyl)-5-(2-oxaspiro[3.3]heptan-6-yl)octahydropyrrolo[3,4-c]pyrrole

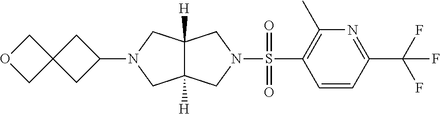 [SMILES: Cc1nc(C(F)(F)F)ccc1S(=O)(=O)N1C[C@H]2CN(C3CC4(COC4)C3)C[C@@H]2C1]

### 1. Synthesis of Rac-tert-butyl (3aR,6aR)-5-((2-methyl-6-(trifluoromethyl)pyridin-3-yl)sulfonyl)hexahydropyrrolo[3,4-c]pyrrole-2(1H)-carboxylate

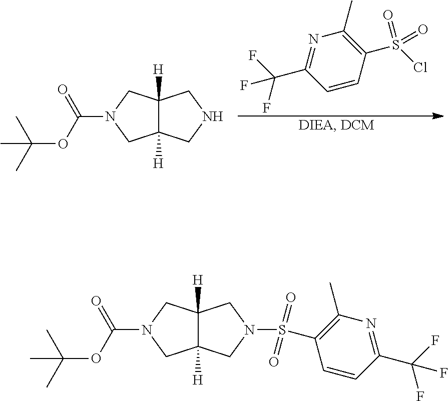

To a solution of Rac-tert-butyl (3aS,6aS)-hexahydropyrrolo[3,4-c]pyrrole-2(1H)-carboxylate (300 mg, 1.41 mmol) and DIEA (547.93 mg, 4.24 mmol) in DCM (15 mL) was added 2-methyl-6-(trifluoromethyl)pyridine-3-sulfonyl chloride (366.91 mg, 1.41 mmol) in DCM (5 mL) at 0° C. The mixture was warmed to 20° C. and stirred for 1 h. The mixture was diluted with water (30 mL) and extracted with DCM (30 mL×3). The combined organic phase was washed with water (20 mL), dried over anhydrous sodium sulfate, filtered and concentrated in vacuo. The crude was purified by flash column (MeOH in DCM=0%˜6%) to give the desired compound (300 mg, 48.7%) as a white solid. LCMS m/z=380.0 [M+H−56]⁺. ¹HNMR (400 MHz, CDCl₃): δ ppm 8.37 (d, J=8.0 Hz, 1H), 7.66 (d, J=8.0 Hz, 1H), 3.70-3.66 (m, 3H), 3.63-3.59 (m, 1H), 3.11 (t, J=10.0 Hz, 2H), 3.02 (t, J=10.0 Hz, 2H), 2.93 (s, 3H), 2.33-2.25 (m, 2H), 1.45 (s, 9H).

### 2. Synthesis of Rac-(3aR,6aR)-2-((2-methyl-6-(trifluoromethyl)pyridin-3-yl)sulfonyl)octahydropyrrolo[3,4-c]pyrrole

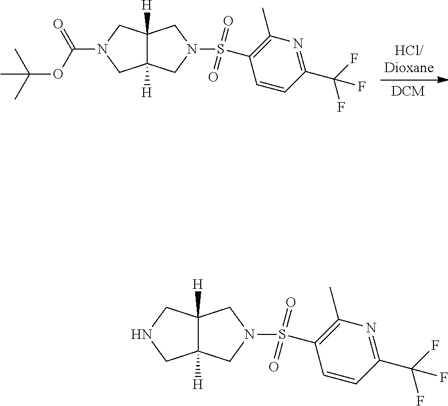 [SMILES: Cc1nc(C(F)(F)F)ccc1S(=O)(=O)N1C[C@H]2CN(C(=O)OC(C)(C)C)C[C@@H]2C1 ; Cc1nc(C(F)(F)F)ccc1S(=O)(=O)N1C[C@H]2CNC[C@@H]2C1]

To a solution of Rac-tert-butyl (3aR,6aR)-5-((2-methyl-6-(trifluoromethyl)pyridin-3-yl)sulfonyl)hexahydropyrrolo[3,4-c]pyrrole-2(1H)-carboxylate (100 mg, 229.64 umol) in DCM (3 mL) was added HCl/Dioxane (1.5 mL), the mixture was stirred at 25° C. for 5 hr. The mixture was concentrated to give the desired compound (85 mg, crude, HCl salt) as a white solid. LCMS m/z=336.0 [M+H]⁺. ¹HNMR (400 MHz, MeOH-d₄): δ ppm 8.47 (d, J=8.0 Hz, 1H), 7.84 (d, J=8.0 Hz, 1H), 3.77-3.72 (m, 2H), 3.53-3.45 (m, 2H), 3.28-3.19 (m, 2H), 3.08-3.00 (m, 2H), 2.91 (s, 3H), 2.68-2.42 (m, 2H).

### 3. Synthesis of Rac-(3aR,6aR)-2-((2-methyl-6-(trifluoromethyl)pyridin-3-yl)sulfonyl)-5-(2-oxaspiro[3.3]heptan-6-yl)octahydropyrrolo[3,4-c]pyrrole

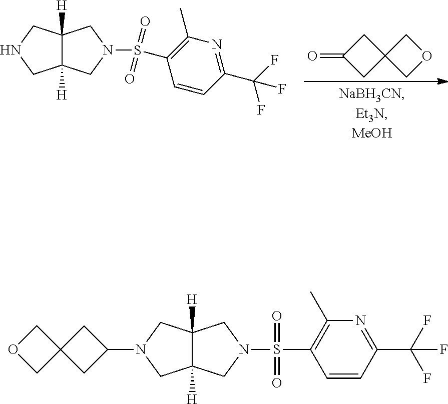

2-Oxaspiro[3.3]heptan-6-one (72.38 mg, 645.50 umol) was added to a solution of Rac-(3aR,6aR)-2-((2-methyl-6-(trifluoromethyl)pyridin-3-yl)sulfonyl)octahydropyrrolo[3,4-c]pyrrole (80 mg, 215.17 umol, HCl salt) and Et₃N (21.77 mg, 215.17 umol) in MeOH (2 mL) (72.38 mg, 645.50 umol) and the solution adjusted to pH=5-6 by addition of acetic acid at 25° C. The solution was stirred at 25° C. for 1 h. The NaBH₃CN (67.61 mg, 1.08 mmol) was added at 25° C., then the mixture was stirred for 6 h. The mixture was neutralized with NH₃·H₂O to pH=7, filtered and purified by prep-HPLC (Column: C18-1 150*30 mm*5 um; Conditions: water (NH₃·H₂O+NH₄HCO₃)-ACN; Begin B: 30; End B: 60; Flow Rate: 25 mL/min) to give the desired compound (30 mg, 32.3%) as a white solid. LCMS m/z=432.1 [M+H]⁺. ¹HNMR (400 MHz, CDCl₃): δ ppm 8.35 (d, J=8.0 Hz, 1H), 7.65 (d, J=8.0 Hz, 1H), 4.68 (s, 2H), 4.61 (s, 2H), 3.66-3.57 (m, 2H), 3.14 (br s, 1H), 3.08-3.01 (m, 2H), 2.92 (s, 3H), 2.87 (br s, 2H), 2.52-2.23 (m, 6H), 2.08 (br s, 2H).

### Example 51: Rac-(3aR,6aR)-2-((2-methyl-6-(trifluoromethyl)pyridin-3-yl)sulfonyl)-5-((tetrahydro-2H-pyran-4-yl)methyl)octahydropyrrolo[3,4-c]pyrrole

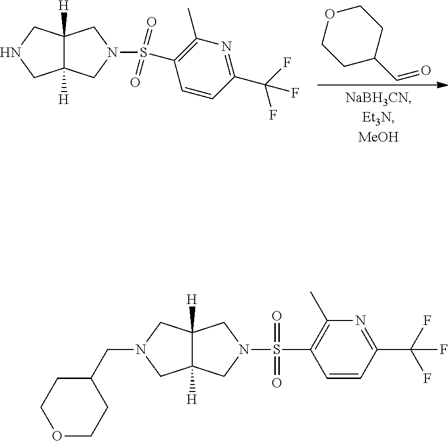

Tetrahydro-2H-pyran-4-carbaldehyde (73.68 mg, 645.50 umol) was added to a solution of Rac-(3aR,6aR)-2-((2-methyl-6-(trifluoromethyl)pyridin-3-yl)sulfonyl)octahydropyrrolo[3,4-c]pyrrole (80 mg, 215.17 umol, HCl salt) and Et₃N (21.77 mg, 215.17 umol) in MeOH (2 mL) and stirred at 25° C. for 1 h. NaBH₃CN (67.61 mg, 1.08 mmol) was added and the mixture was stirred at 25° C. for 6 h. The mixture was neutralized with NH₃·H₂O to pH=7, filtered and purified by prep-HPLC (Column: C18-1 150*30 mm*5 um; Conditions: water (NH₃·H₂O+NH₄HCO₃)-ACN; Begin B: 36; End B: 66; Flow Rate: 25 mL/min) to give the desired compound (30 mg, 32.1%) as a white solid. LCMS m/z=434.1 [M+H]⁺. ¹HNMR (400 MHz, CDCl₃): δ ppm 8.35 (d, J=8.0 Hz, 1H), 7.65 (d, J=8.0 Hz, 1H), 4.01-3.92 (m, 2H), 3.67-3.58 (m, 2H), 3.41-3.31 (m, 2H), 3.11-3.01 (m, 2H), 2.92 (s, 3H), 2.88 (br s, 2H), 2.57 (br s, 4H), 2.34 (br s, 2H), 1.65-1.60 (m, 3H), 1.33-1.19 (m, 2H).

### Example 52: Rac-(3aR,6aR)-2-((2-methyl-6-(trifluoromethyl)pyridin-3-yl)sulfonyl)-5-(oxetan-3-ylmethyl)octahydropyrrolo[3,4-c]pyrrole

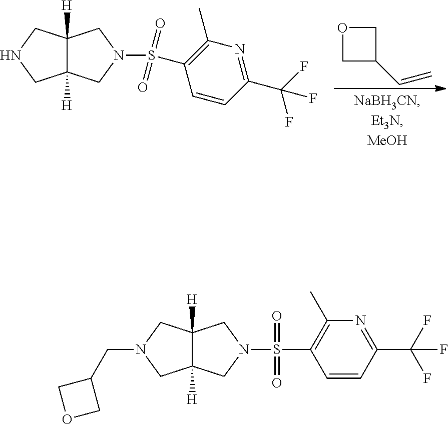

Rac-(3aR,6aR)-2-((2-methyl-6-(trifluoromethyl)pyridin-3-yl)sulfonyl)-5-(oxetan-3-ylmethyl)octahydropyrrolo[3,4-c]pyrrole was obtained (20 mg, yield 23%) as a white solid, from Rac-(3aR,6aR)-2-((2-methyl-6-(trifluoromethyl)pyridin-3-yl)sulfonyl)octahydropyrrolo[3,4-c]pyrrole and oxetane-3-carbaldehyde, following a similar procedure described in Example 51. LCMS m/z=406.1 [M+H]⁺. ¹HNMR (400 MHz, CDCl₃): δ ppm 8.35 (d, J=8.0 Hz, 1H), 7.64 (d, J=8.0 Hz, 1H), 4.82-4.72 (m, 2H), 4.42-4.34 (m, 2H), 3.66-3.58 (m, 2H), 3.13-2.96 (m, 5H), 2.92 (s, 3H), 2.86-2.79 (m, 2H), 2.60-2.52 (m, 2H), 2.37-2.23 (m, 2H).

### Example 53: Rac-(3aR,6aR)-2-((1-methyl-2-oxabicyclo[3.1.1]heptan-5-yl)methyl)-5-((2-methyl-6-(trifluoromethyl)pyridin-3-yl)sulfonyl)octahydropyrrolo[3,4-c]pyrrole

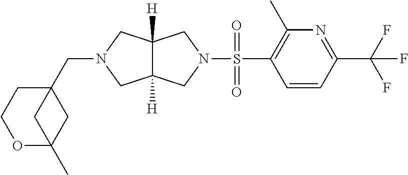 [SMILES: Cc1nc(C(F)(F)F)ccc1S(=O)(=O)N1C[C@H]2CN(CC34CCOC(C)(C3)C4)C[C@@H]2C1]

### 1. Synthesis of Rac-((3aR,6aR)-5-((2-methyl-6-(trifluoromethyl)pyridin-3-yl)sulfonyl)hexahydropyrrolo[3,4-c]pyrrol-2(H)-yl)(1-methyl-2-oxabicyclo[3.1.1]heptan-5-yl)methanone

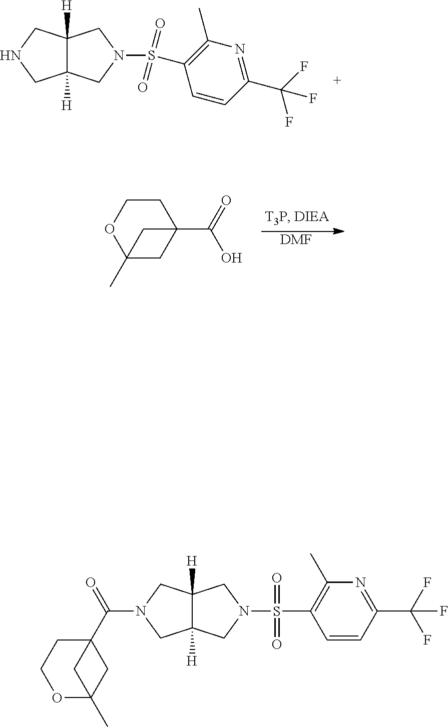

A mixture of Rac-(3aR,6aR)-2-((2-methyl-6-(trifluoromethyl)pyridin-3-yl)sulfonyl)octahydropyrrolo[3,4-c]pyrrole (150.00 mg, 447.30 umol) and T3P (426.97 mg, 670.95 umol, 399.41 uL, 50% purity) was dissolved in DMF (2 mL) and DIPEA (289.05 mg, 2.24 mmol, 389.55 uL) and heated at 80° C. for 10 min. To the above orange mixture was added a solution of 1-methyl-2-oxabicyclo[3.1.1]heptane-5-carboxylic acid (83.83 mg, 536.76 umol) in DMF (2 mL) and the reaction stirred an additional 1 hr at 80° C., then cooled to RT and stirred overnight. The reaction mixture was diluted with DCM (5 mL) and washed with sat. aq. NaHCO₃, water and brine. The organic phase was dried over Na₂SO₄, filtered and concentrated.

Crude material was purified by column chromatography (12 g SiO₂, 50-100% EtOH:EtOAc 1:3 in heptane) to afford the desired compound (152 mg, 72%). LCMS m/z=474.0 [M+H]⁺. ¹HNMR (400 MHz, CDCl₃): δ 8.39 (br d, 1H, J=8.2 Hz), 7.68 (br d, 1H, J=8.2 Hz), 4.1-4.2 (m, 2H), 3.84 (br dd, 1H, J=6.9, 10.8 Hz), 3.7-3.8 (m, 3H), 3.1-3.2 (m, 4H), 2.9-3.0 (m, 3H), 2.3-2.4 (m, 1H), 2.1-2.3 (m, 6H), 2.0-2.1 (m, 2H), 1.25 (s, 3H).

### 2. Synthesis of Rac-(3aR,6aR)-2-((1-methyl-2-oxabicyclo[3.1.1]heptan-5-yl)methyl)-5-((2-methyl-6-(trifluoromethyl)pyridin-3-yl)sulfonyl)octahydropyrrolo[3,4-c]pyrrole

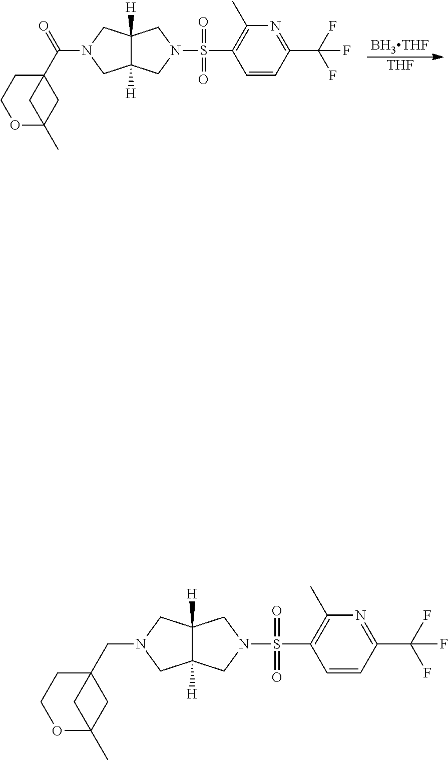 [SMILES: Cc1nc(C(F)(F)F)ccc1S(=O)(=O)N1C[C@H]2CN(C(=O)C34CCOC(C)(C3)C4)C[C@@H]2C1 ; "BH3·THF" ; "THF" ; Cc1nc(C(F)(F)F)ccc1S(=O)(=O)N1C[C@H]2CN(CC34CCOC(C)(C3)C4)C[C@@H]2C1]

Rac-[(3aS,6aS)-5-[[2-methyl-6-(trifluoromethyl)-3-pyridyl]sulfonyl]-1,3,3a,4,6,6a-hexahydropyrrolo[3,4-c]pyrrol-2-yl]-(1-methyl-2-oxabicyclo[3.1.1]heptan-5-yl)methanone (135.28 mg, 285.70 umol) was dissolved in THE (1 mL). 1M borane in THF (1.14 ml, 1.14 mmol) was added dropwise and the solution heated to 50° C. overnight. The reaction was cooled to RT and quenched with addition of 5 ml MeoH. The reaction was heated back up to 50° C. and stirred overnight. The reaction was then cooled to RT and the solvent removed in vacuo. Crude product was purified by column chromatography (12 g SiO₂, 50-100% EtOH:EtOAc 1:3 in heptane) to afford the desired compound (102 mg, 78%). LCMS m/z=474.0 [M+H]⁺. ¹HNMR (400 MHz, CDCl₃): ¹H NMR (CHLOROFORM-d, 500 MHz): δ 8.29 (br d, 1H, J=7.9 Hz), 7.58 (d, 1H, J=7.9 Hz), 3.98 (t, 2H, J=6.9 Hz), 3.5-3.6 (m, 2H), 2.99 (br t, 2H, J=9.5 Hz), 2.86 (s, 3H), 2.79 (br s, 2H), 2.5-2.7 (m, 4H), 2.26 (br s, 2H), 1.87 (br s, 2H), 1.7-1.8 (m, 2H), 1.52 (br dd, 2H, J=7.8, 17.2 Hz), 1.15 (s, 3H).

### Example 54: Rac-(3aR,6aR)-3a-methyl-2-((1-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole

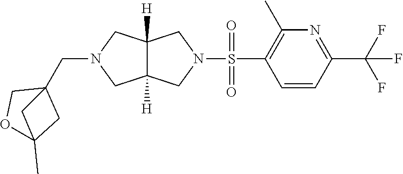 [SMILES: Cc1nc(C(F)(F)F)ccc1S(=O)(=O)N1C[C@H]2CN(CC34COC(C)(C3)C4)C[C@@H]2C1]

### 1. Synthesis of Rac-((3aR,6aR)-5-((2-methyl-6-(trifluoromethyl)pyridin-3-yl)sulfonyl)hexahydropyrrolo[3,4-c]pyrrol-2(H)-yl)(1-methyl-2-oxabicyclo[2.1.1]hexan-4-yl)methanone

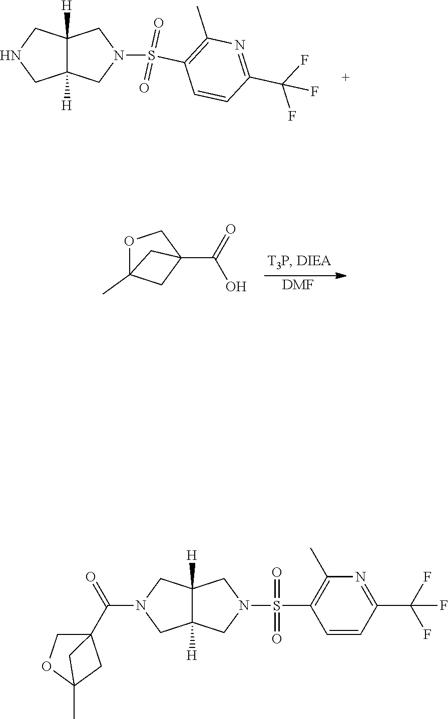

Rac-((3aR,6aR)-5-((2-methyl-6-(trifluoromethyl)pyridin-3-yl)sulfonyl)hexahydropyrrolo[3,4-c]pyrrol-2(1H)-yl)(1-methyl-2-oxabicyclo[2.1.1]hexan-4-yl)methanone was obtained from Rac-(3aS,6aS)-5-[[2-methyl-6-(trifluoromethyl)-3-pyridyl]sulfonyl]-2,3,3a,4,6,6a-hexahydro-1H-pyrrolo[3,4-c]pyrrole (150 mg, 447.30 umol) and 1-methyl-2-oxabicyclo[2.1.1]hexane-4-carboxylic acid (76.30 mg, 536.76 umol) following a similar procedure described in Example 53 step 1. Crude material was purified by column chromatography (12 g SiO₂, 50-100% EtOH:EtOAc 1:3 in heptane) to afford the desired material (165 mg, 80%). LCMS m/z=460.1 [M+H]⁺. ¹H NMR (CHLOROFORM-d, 500 MHz): ¹H NMR ¹H NMR (CHLOROFORM-d, 500 MHz) δ 8.39 (br d, 1H, J=7.9 Hz), 7.68 (br d, 1H, J=7.9 Hz), 3.8-4.0 (m, 3H), 3.7-3.8 (m, 3H), 3.0-3.3 (m, 4H), 2.93 (s, 3H), 2.2-2.5 (m, 2H), 1.8-2.0 (m, 4H), 1.45 (s, 3H).

### 2. Synthesis of Rac-(3aR,6aR)-2-((I-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole

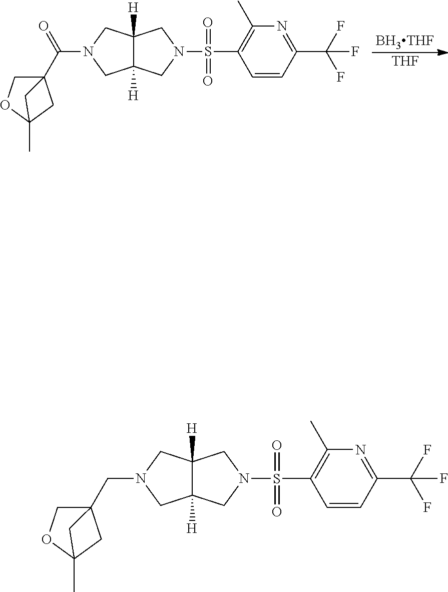 [SMILES: Cc1nc(C(F)(F)F)ccc1S(=O)(=O)N1C[C@H]2CN(C(=O)C34COC(C)(C3)C4)C[C@@H]2C1 ; Cc1nc(C(F)(F)F)ccc1S(=O)(=O)N1C[C@H]2CN(CC34COC(C)(C3)C4)C[C@@H]2C1 ; "THF" ; "BH3·THF"]

Rac-(3aR,6aR)-2-((1-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole (74 mg, 76%) was obtained from Rac-((3aR,6aR)-5-((2-methyl-6-(trifluoromethyl)pyridin-3-yl)sulfonyl)hexahydropyrrolo[3,4-c]pyrrol-2(1H)-yl)(1-methyl-2-oxabicyclo[2.1.1]hexan-4-yl)methanone following a similar procedure described in Example 53 step 2. LCMS m/z=446.2 [M+H]⁺. ¹H NMR (CHLOROFORM-d, 500 MHz): δ 8.28 (br d, 1H, J=7.9 Hz), 7.58 (br d, 1H, J=8.2 Hz), 3.4-3.7 (m, 4H), 2.7-3.1 (m, 7H), 2.2-2.7 (m, 4H), 1.4-1.7 (m, 6H), 1.35 (s, 3H).

### Examples 55 and 56: (3aS,6aS)-2-((1-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole and (3aR,6aR)-3a-methyl-2-((1-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole

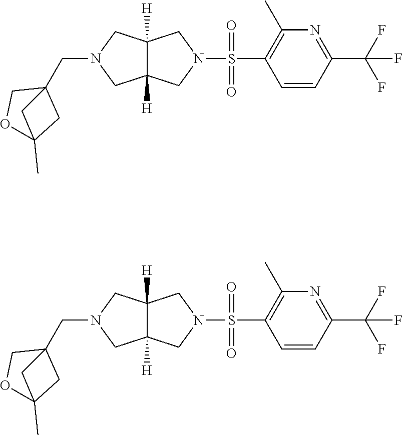 [SMILES: Cc1nc(C(F)(F)F)ccc1S(=O)(=O)N1C[C@@H]2CN(CC34COC(C)(C3)C4)C[C@H]2C1 ; Cc1nc(C(F)(F)F)ccc1S(=O)(=O)N1C[C@H]2CN(CC34COC(C)(C3)C4)C[C@@H]2C1]

Rac-(3aR,6aR)-3a-methyl-2-((1-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole was resolved by chiral SFC purification using a CHIRALPAK OX—H 30×250 mm, Sum. Method: 20% IPA w/0.1% DEA in C₀₂ (flow rate: 100 mL/min, ABPR 120 bar, MBPR 60 psi, column temp 40 deg C.) to afford: (3aS,6aS)-2-((1-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole (19.7 mg, 31.8%, Rf=2.33 min.). LCMS m/z=446.2 [M+H]⁺. Compound chirality was assigned arbitrarily. (3aR,6aR)-3a-methyl-2-((1-methyl-2-oxabicyclo[2.1.1]hexan-4-yl)methyl)-5-((2-methyl-6-(trifluoromethyl)pyridin-3-yl)sulfonyl)octahydropyrrolo[3,4-c]pyrrole (20.2 mg, 32.6%, Rf=2.52 min.). LCMS m/z=446.2 [M+H]⁺. Compound chirality was assigned arbitrarily.

### Example 57: Rac-(3aR,6aR)-2-((4-methyl-2-(trifluoromethyl)pyrimidin-5-yl)sulfonyl)-5-(tetrahydro-2H-pyran-4-yl)octahydropyrrolo[3,4-c]pyrrole

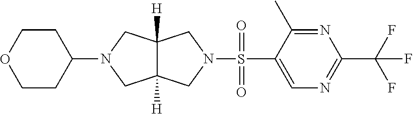 [SMILES: Cc1nc(C(F)(F)F)ncc1S(=O)(=O)N1C[C@H]2CN(C3CCOCC3)C[C@@H]2C1]

### 1. Synthesis of Rac-tert-butyl (3aR,6aR)-5-((4-methyl-2-(trifluoromethyl)pyrimidin-5-yl)sulfonyl)hexahydropyrrolo[3,4-c]pyrrole-2(1H)-carboxylate

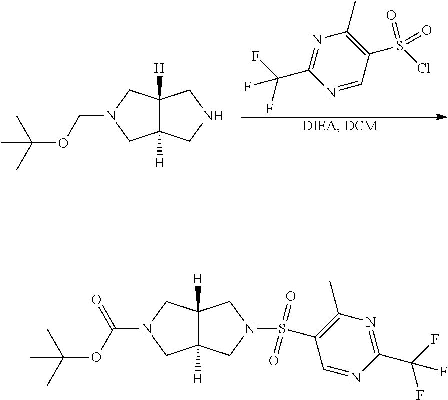

To a solution of 4-methyl-2-(trifluoromethyl)pyrimidine-5-sulfonyl chloride (200 mg, 780.34 umol, 0.5 Oxalate salt) and DIPEA (302.56 mg, 2.34 mmol) in DCM (10 mL) was added tert-butyl Rac-(3aS,6aS)-hexahydropyrrolo[3,4-c]pyrrole-2(1H)-carboxylate (350 mg, 1.34 mmol) at 0° C. The mixture was stirred at 20° C. for 2 hours. The mixture was concentrated to give the crude residue, which was purified by silica gel chromatography (Petroleum ether in EtOAc from 0% to 30%) to give the desired compound (190 mg, 55.79%) as yellow solid. LCMS m/z=381.0 [M+H-t-Bu]⁺. ¹H NMR (CHLOROFORM-d, 500 MHz): δ ppm 9.18 (s, 1H), 3.78-3.61 (m, 4H), 3.20-3.13 (m, 2H), 3.03 (br t, J=10.4 Hz, 2H), 2.95 (s, 3H), 2.37-2.23 (m, 2H), 1.46 (s, 9H).

### 2. Synthesis of Rac-(3aR,6aR)-2-((4-methyl-2-(trifluoromethyl)pyrimidin-5-yl)sulfonyl)octahydropyrrolo[3,4-c]pyrrole

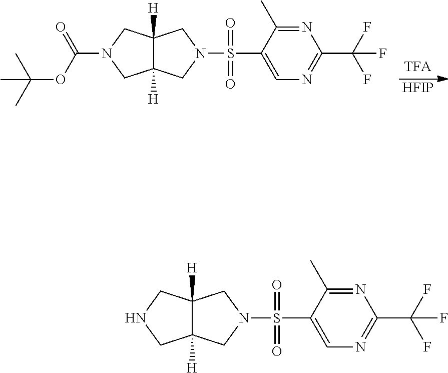

To a solution of Rac-tert-butyl (3aR,6aR)-5-((4-methyl-2-(trifluoromethyl)pyrimidin-5-yl)sulfonyl)hexahydropyrrolo[3,4-c]pyrrole-2(1H)-carboxylate (180 mg, 412.42 umol) in HFIP (5 mL) was added TFA (141.08 mg, 1.24 mmol, 94.75 uL) at 20° C. The mixture was stirred at 20° C. for 2 hours. The mixture was concentrated to give the desired compound (180 mg, crude, TFA salt) as light yellow solid. LCMS m/z=337.0 [M+H]⁺. ¹H NMR (CHLOROFORM-d, 500 MHz): δ ppm 10.04-9.95 (m, 1H), 9.19 (s, 1H), 3.85-3.78 (m, 2H), 3.65-3.54 (m, 2H), 3.33-3.20 (m, 2H), 3.13-3.03 (m, 2H), 2.94 (s, 3H), 2.64-2.57 (m, 2H).

### 3. Synthesis of Rac-(3aR,6aR)-2-((4-methyl-2-(trifluoromethyl)pyrimidin-5-yl)sulfonyl)-5-(tetrahydro-2H-pyran-4-yl)octahydropyrrolo[3,4-c]pyrrole

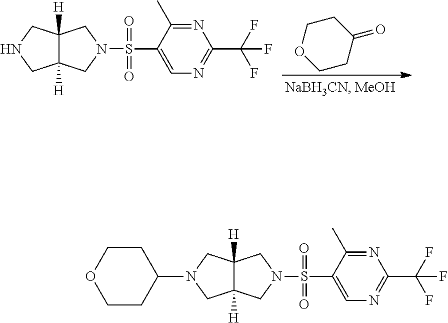

Rac-(3aR,6aR)-2-((4-methyl-2-(trifluoromethyl)pyrimidin-5-yl)sulfonyl)-5-(tetrahydro-2H-pyran-4-yl)octahydropyrrolo[3,4-c]pyrrole was obtained (28 mg, yield 48%) as a white solid, from Rac-(3aR,6aR)-2-((4-methyl-2-(trifluoromethyl)pyrimidin-5-yl)sulfonyl)octahydropyrrolo[3,4-c]pyrrole and tetrahydro-4H-pyran-4-one, following a similar procedure described in Example 51. LCMS m/z=421.2 [M+H]⁺. ¹HNMR (400 MHz, CDCl₃): δ ppm 9.17 (s, 1H), 3.99 (br d, J=11.2 Hz, 2H), 3.72-3.65 (m, 2H), 3.43-3.32 (m, 2H), 3.16-3.10 (m, 2H), 3.05-2.98 (m, 2H), 2.95 (s, 3H), 2.81-2.72 (m, 1H), 2.71-2.62 (m, 2H), 2.39-2.26 (m, 2H), 1.82-1.72 (m, 2H), 1.64-1.50 (m, 2H).

### Example 58: Rac-(3aR,6aR)-2-((4-methyl-2-(trifluoromethyl)pyrimidin-5-yl)sulfonyl)-5-(2-oxaspiro[3.3]heptan-6-yl)octahydropyrrolo[3,4-c]pyrrole

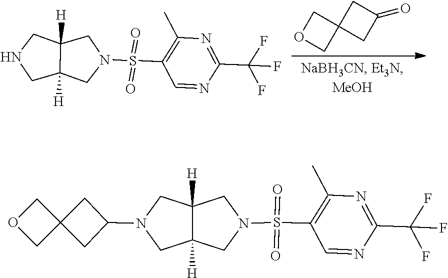

Rac-(3aR,6aR)-2-((4-methyl-2-(trifluoromethyl)pyrimidin-5-yl)sulfonyl)-5-(2-oxaspiro[3.3]heptan-6-yl)octahydropyrrolo[3,4-c]pyrrole was obtained (21.6 mg, yield 36.7%) as a white solid, from Rac-(3aR,6aR)-2-((4-methyl-2-(trifluoromethyl)pyrimidin-5-yl)sulfonyl)octahydropyrrolo[3,4-c]pyrrole and 2-oxaspiro[3.3]heptan-6-one, following a similar procedure described in Example 51. LCMS m/z=433.1 [M+H]⁺. ¹HNMR (400 MHz, CDCl₃): δ ppm 9.18 (s, 1H), 4.71 (s, 2H), 4.63 (s, 2H), 3.74-3.64 (m, 2H), 3.22-3.07 (m, 3H), 2.96 (s, 3H), 2.93-2.82 (m, 2H), 2.56-2.25 (m, 6H), 2.21-1.98 (m, 2H).

### Example 59: Rac-(3aR,6aR)-2-((4-methyl-2-(trifluoromethyl)pyrimidin-5-yl)sulfonyl)-5-((tetrahydro-2H-pyran-4-yl)methyl)octahydropyrrolo[3,4-c]pyrrole

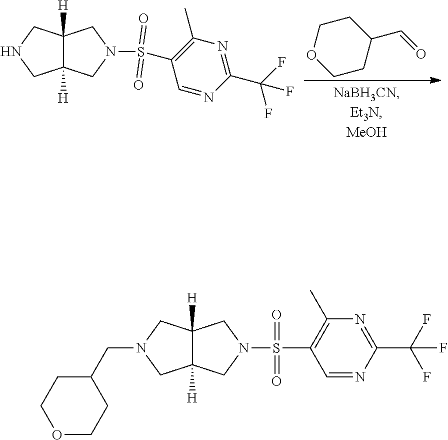

(3aR,6aR)-2-((4-methyl-2-(trifluoromethyl)pyrimidin-5-yl)sulfonyl)-5-((tetrahydro-2H-pyran-4-yl)methyl)octahydropyrrolo[3,4-c]pyrrole was obtained (22 mg, yield 37.5%) as a white solid, from Rac-(3aR,6aR)-2-((4-methyl-2-(trifluoromethyl)pyrimidin-5-yl)sulfonyl)octahydropyrrolo[3,4-c]pyrrole and tetrahydro-2H-pyran-4-carbaldehyde, following a similar procedure described in Example 51. LCMS m/z=435.1 [M+H]⁺. ¹HNMR (400 MHz, CDCl₃): δ ppm 9.16 (s, 1H), 3.98-3.94 (m, 2H), 3.68-3.67 (m, 2H), 3.36 (td, J=12.0 Hz, 1.6 Hz, 2H), 3.17-3.06 (m, 2H), 2.95 (s, 3H), 2.93-2.83 (m, 2H), 2.69-2.51 (m, 4H), 2.28-2.43 (m, 2H), 1.70-1.62 (m, 3H), 1.31-1.20 (m, 2H).

### Example 60: (3aR,6aR)-2-((4,6-dimethyl-2-(trifluoromethyl)pyrimidin-5-yl)sulfonyl)-5-(tetrahydro-2H-pyran-4-yl)octahydropyrrolo[3,4-c]pyrrole

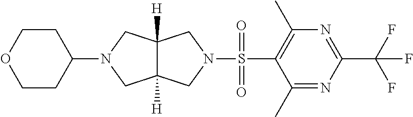 [SMILES: Cc1nc(C(F)(F)F)nc(C)c1S(=O)(=O)N1C[C@H]2CN(C3CCOCC3)C[C@@H]2C1]

### 1. Synthesis of tert-butyl (3aR,6aR)-5-(tetrahydro-2H-pyran-4-yl)hexahydropyrrolo[3,4-c]pyrrole-2(1H)-carboxylate

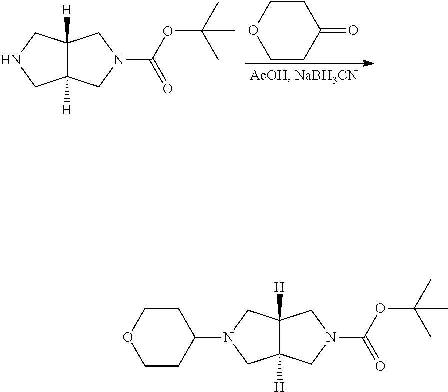

tert-butyl (3aR,6aR)-5-(tetrahydro-2H-pyran-4-yl)hexahydropyrrolo[3,4-c]pyrrole-2(1H)-carboxylate was obtained (300 mg, yield 86.0%) from tert-butyl (3aR,6aR)-hexahydropyrrolo[3,4-c]pyrrole-2(1H)-carboxylate and tetrahydro-4H-pyran-4-one, following a similar procedure described in Example 34 step 1. ¹HNMR (400 MHz, CDCl₃): δ ppm 3.97-3.95 (m, 2H), 3.88-3.85 (m, 2H), 3.67-3.64 (m, 2H), 3.45-3.44 (m, 2H), 3.43-3.38 (m, 3H), 1.91-1.90 (m, 2H), 1.61-1.59 (m, 2H), 1.57-1.55 (m, 4H), 1.46 (s, 9H).

### 2. Synthesis of (3aS,6aS)-2-(tetrahydro-2H-pyran-4-yl)octahydropyrrolo[3,4-c]pyrrole

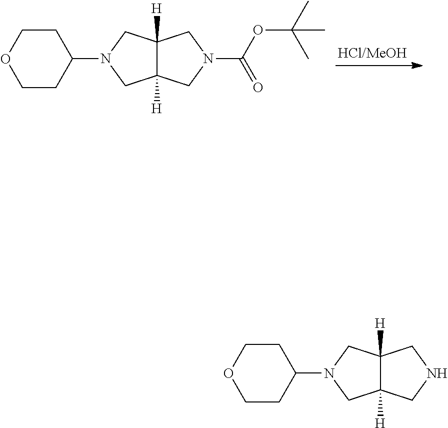 [SMILES: CC(C)(C)OC(=O)N1C[C@H]2CN(C3CCOCC3)C[C@@H]2C1 ; C1CC(N2C[C@@H]3CNC[C@H]3C2)CCO1]

(3aS,6aS)-2-(Tetrahydro-2H-pyran-4-yl)octahydropyrrolo[3,4-c]pyrrole was obtained (80 mg, 100%, HCl salt) from tert-butyl (3aR,6aR)-5-(tetrahydro-2H-pyran-4-yl)hexahydropyrrolo[3,4-c]pyrrole-2(1H)-carboxylate and HCl in MeoH, following a similar procedure described in Example 34 step 2. ¹HNMR (400 MHz, CDCl₃): δ ppm 4.17-3.99 (m, 2H), 3.97-3.87 (m, 1H), 3.84-3.70 (m, 2H), 3.62-3.55 (m, 2H), 3.48-3.43 (m, 2H), 3.22-3.11 (m, 2H), 2.89-2.57 (m, 2H), 2.17-1.98 (m, 2H), 1.93-1.68 (m, 3H), 1.59-1.49 (m, 1H).

### 3. Synthesis of 4,6-dibromo-2-(trifluoromethyl)pyrimidin-5-amine

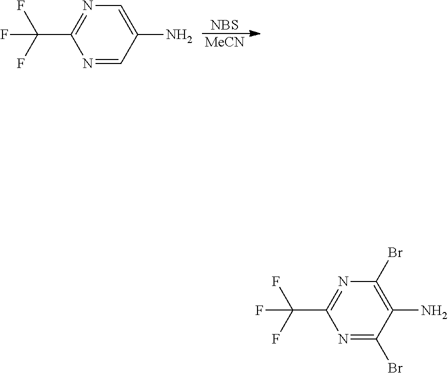

To a solution of 2-(trifluoromethyl)pyrimidin-5-amine (2.5 g, 15.33 mmol) in MeCN (40 mL) was added NBS (6.00 g, 33.72 mmol) at 20° C. The mixture was stirred at 40° C. for 12 hours. The mixture was concentrated to give the crude residue, which was purified by silica gel chromatography (Petroleum ether/EtOAc from 0% to 30%) to give the desired compound (2.5 g, 50.83% yield) as yellow solid. LCMS m/z=321.6 [M+H]⁺.

### 4. Synthesis of 4,6-dimethyl-2-(trifluoromethyl)pyrimidin-5-amine

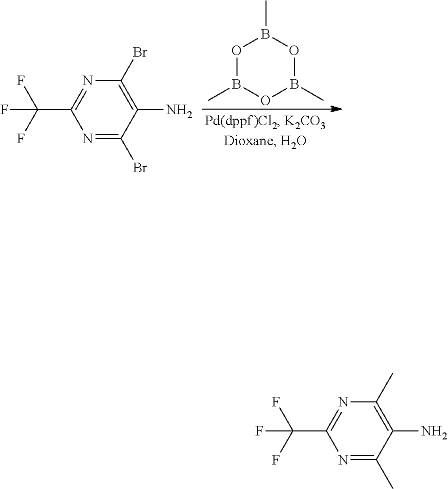

To a solution of 4,6-dibromo-2-(trifluoromethyl)pyrimidin-5-amine (7.82 g, 31.16 mmol) and 2,4,6-trimethyl-1,3,5,2,4,6-trioxatriborinane (2.5 g, 7.79 mmol) in Dioxane (100 mL) and water (20 mL) was added Pd(dppf)Cl2 (570.05 mg, 779.08 umol) and K2CO3 (2.69 g, 19.48 mmol) at 20° C. The mixture was stirred at 80° C. for 12 hours under N2. The mixture was concentrated to give the crude residue, which was purified by silica gel chromatography (EtOAc in Petroleum ether from 0% to 30%) to give the desired compound (1.4 g, 94.0% yield) as a yellow solid. LCMS m/z=192.1 [M+H]⁺. ¹HNMR (400 MHz, CDCl₃): δ ppm 4.04-3.50 (m, 2H), 2.46 (s, 6H).

### 5. Synthesis of 5-bromo-4,6-dimethyl-2-(trifluoromethyl)pyrimidine

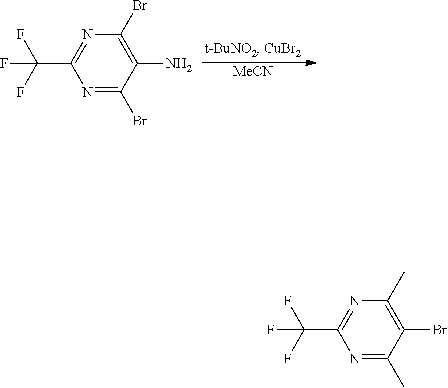

To a solution of 4,6-dimethyl-2-(trifluoromethyl)pyrimidin-5-amine (1.4 g, 7.32 mmol) in MeCN (5 mL) was added CuBr₂ (2.45 g, 10.99 mmol) and tert-butyl nitrite (1.13 g, 10.99 mmol). The mixture was stirred at 60° C. for 1 hour. The mixture was concentrated to give the crude residue, which was purified by silica gel chromatography (EtOAc in Petroleum ether from 0% to 10%) to give the desired compound (1 g, 53% yield) as colorless oil. LCMS m/z=254.7 [M+H]⁺. T HNMR (400 MHz, CDCl₃): δ ppm 2.74 (s, 6H).

### 6. Synthesis of 5-(benzylthio)-4,6-dimethyl-2-(trifluoromethyl)pyrimidine

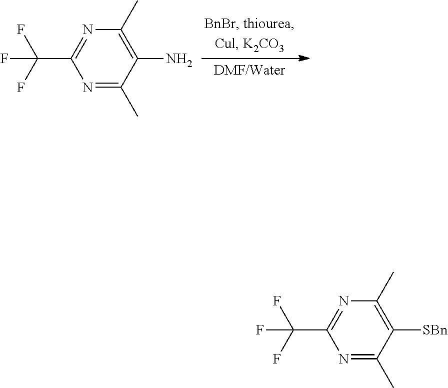

To a 20° C. solution of BnBr (905.35 mg, 5.29 mmol) and 5-bromo-4,6-dimethyl-2-(trifluoromethyl)pyrimidine (900 mg, 3.53 mmol) in DMF (40 mL) and water (0.2 mL) was added K2CO3 (1.95 g, 14.12 mmol), Thiourea (671.56 mg, 8.82 mmol) and CuI (100.81 mg, 529.34 mol). The mixture was heated to 60° C. and stirred for 0.5 h. The mixture was diluted with water (200 mL) and extracted with EtOAc (50 mL×3). The combined organic layers were dried over Na₂SO₄, filtered and concentrated to give the crude residue, which was purified by silica gel chromatography (EtOAc in Petroleum ether from 0% to 10%) to give the desired compound (0.8 g, 76% yield) as a yellow oil. LCMS m/z=298.9 [M+H]⁺. ¹HNMR (400 MHz, CDCl₃): δ ppm 7.28-7.19 (m, 3H), 7.17-7.15 (m, 2H), 3.82 (s, 2H), 2.53 (s, 6H).

### 7. Synthesis of 4,6-dimethyl-2-(trifluoromethyl)pyrimidine-5-sulfonyl chloride

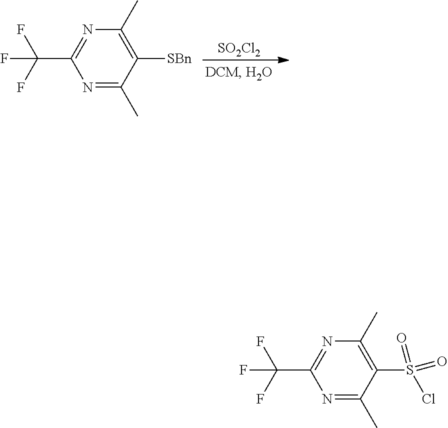

A sulfuryl chloride (2.00 g, 14.78 mmol) in DCM (2 mL) was added to a 0° C. solution of 5-(benzylthio)-4,6-dimethyl-2-(trifluoromethyl)pyrimidine (630 mg, 2.11 mmol) in water (4 mL) and DCM (20 mL). The reaction mixture was stirred at 0-15° C. for 12 h. The mixture was diluted with water (100 mL) and extracted with DCM (40 mL×3). The aqueous phase was neutralized with NaHCO₃ and the combined organic layers were dried over anhydrous sodium sulfate, filtered, and concentrated in vacuo to give the desired compound (600 mg, crude) as a yellow solid. ¹HNMR (400 MHz, CDCl3) δ ppm 3.07 (s, 6H).

### 8. Synthesis of (3aR,6aR)-2-((4,6-dimethyl-2-(trifluoromethyl)pyrimidin-5-yl)sulfonyl)-5-(tetrahydro-2H-pyran-4-yl)octahydropyrrolo[3,4-c]pyrrole

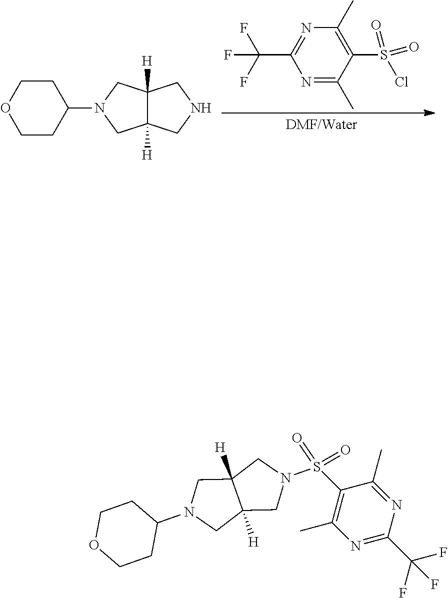 [SMILES: C1CC(N2C[C@@H]3CNC[C@H]3C2)CCO1 ; Cc1nc(C(F)(F)F)nc(C)c1S(=O)(=O)Cl ; Cc1nc(C(F)(F)F)nc(C)c1S(=O)(=O)N1C[C@H]2CN(C3CCOCC3)C[C@@H]2C1]

4,6-Dimethyl-2-(trifluoromethyl)pyrimidine-5-sulfonyl chloride (129.80 mg, 472.61 umol) was added to a solution of (3aS,6aS)-2-(tetrahydro-2H-pyran-4-yl)octahydropyrrolo[3,4-c]pyrrole (100 mg, 429.65 umol, HCl salt) and DIEA (166.58 mg, 1.29 mmol, 224.50 uL) in DCM (10 mL). The mixture was stirred at 20° C. for 2 hours. The mixture was filtered and concentrated in vacuum. The crude material was purified by prep-HPLC (Column:Welch Xtimate C18 150*25 mm*5 um; Condition: water (NH₄HCO₃)-ACN; Begin B: 31; End B: 61; Flow Rate (ml/min): 25) to give the desired compound (50 mg, 27% yield) as a white solid. LCMS m/z=435.1 [M+H]⁺. T HNMR (500 MHz, MeOD): δ ppm 4.97-4.93 (m, 2H), 4.67-4.63 (m, 2H), 4.00-3.96 (m, 2H), 3.10-3.08 (m, 2H), 2.99-2.97 (m, 2H), 2.93 (s, 6H), 2.77-2.73 (m, 1H), 2.62-2.58 (m, 2H), 2.30-2.28 (m, 2H), 1.83-1.80 (m, 2H), 1.53-1.48 (m, 2H).

### Example 61: (3aR,6aR)-2-((4,6-dimethyl-2-(trifluoromethyl)pyrimidin-5-yl)sulfonyl)-5-(2-oxaspiro[3.3]heptan-6-yl)octahydropyrrolo[3,4-c]pyrrole

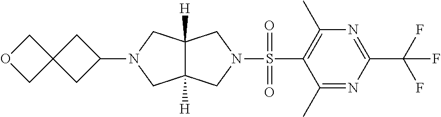 [SMILES: Cc1nc(C(F)(F)F)nc(C)c1S(=O)(=O)N1C[C@H]2CN(C3CC4(COC4)C3)C[C@@H]2C1]

### 1. Synthesis of tert-butyl (3aR,6aR)-5-((4,6-dimethyl-2-(trifluoromethyl)pyrimidin-5-yl)sulfonyl)hexahydropyrrolo[3,4-c]pyrrole-2(1H)-carboxylate

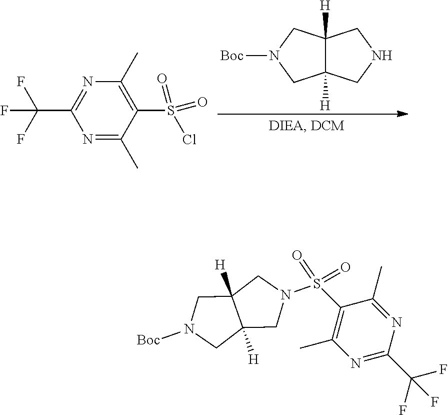 [SMILES: Cc1nc(C(F)(F)F)nc(C)c1S(=O)(=O)Cl ; CC(C)(C)OC(=O)N1C[C@@H]2CNC[C@H]2C1 ; Cc1nc(C(F)(F)F)nc(C)c1S(=O)(=O)N1C[C@H]2CN(C(=O)OC(C)(C)C)C[C@@H]2C1]

tert-Butyl (3aS,6aS)-hexahydropyrrolo[3,4-c]pyrrole-2(1H)-carboxylate (274.37 mg, 998.98 umol) was added to a solution of 4,6-dimethyl-2-(trifluoromethyl)pyrimidine-5-sulfonyl chloride (200 mg, 665.99 umol, Oxalate salt) and DIEA (258.22 mg, 2.00 mmol, 348.00 uL) in DCM (10 mL). The mixture was stirred at 20° C. for 2 hours. The mixture was concentrated to give the crude residue, which was purified by silica gel chromatography (Petroleum ether/EtOAc=5/1 to 3/1) to give the desired product (110 mg, 244.19 umol, 36.7% yield) as a yellow solid. LCMS m/z=395.1 [M-tBu+H]⁺. ¹HNMR (500 MHz, MeOD): δ ppm 3.74-3.70 (m, 2H), 3.64-3.61 (m, 2H), 3.19-3.15 (m, 2H), 3.07-3.03 (m, 2H), 2.95 (s, 6H), 2.38-2.35 (m, 2H), 1.47 (s, 9H).

### 2. Synthesis of (3aR,6aR)-2-((4,6-dimethyl-2-(trifluoromethyl)pyrimidin-5-yl)sulfonyl)octahydropyrrolo[3,4-c]pyrrole

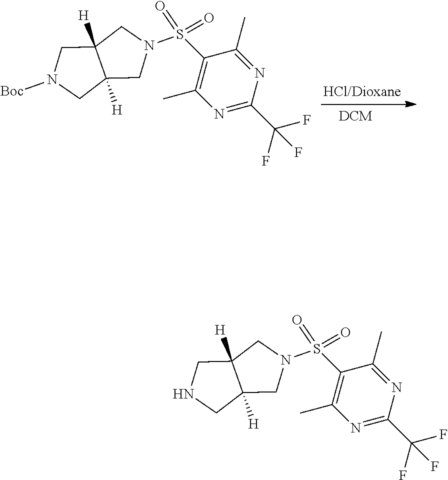 [SMILES: Cc1nc(C(F)(F)F)nc(C)c1S(=O)(=O)N1C[C@H]2CN(C(=O)OC(C)(C)C)C[C@@H]2C1 ; Cc1nc(C(F)(F)F)nc(C)c1S(=O)(=O)N1C[C@H]2CNC[C@@H]2C1]

To a solution of tert-butyl (3aR,6aR)-5-((4,6-dimethyl-2-(trifluoromethyl)pyrimidin-5-yl)sulfonyl)hexahydropyrrolo[3,4-c]pyrrole-2(1H)-carboxylate (110 mg, 244.19 umol) in DCM (3 mL) was added HCl/Dioxane (4 M, 3 mL). The mixture was stirred at 20° C. for 1 h. The mixture was concentrated to give compound 4 (120 mg, HCl salt) as a yellow solid. LCMS m/z=351.1 [M+H]⁺.

### 3. Synthesis of (3aR,6aR)-2-((4,6-dimethyl-2-(trifluoromethyl)pyrimidin-5-yl)sulfonyl)-5-(2-oxaspiro[3.3]heptan-6-yl)octahydropyrrolo[3,4-c]pyrrole

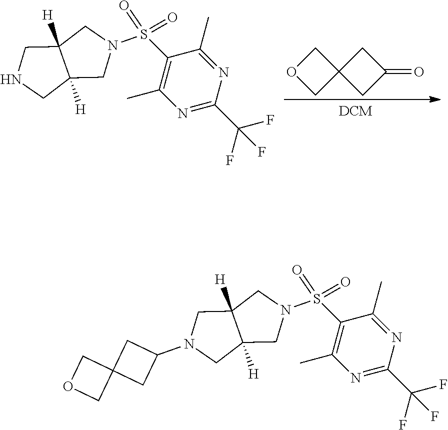 [SMILES: Cc1nc(C(F)(F)F)nc(C)c1S(=O)(=O)N1C[C@H]2CNC[C@@H]2C1 ; O=C1CC2(COC2)C1 ; Cc1nc(C(F)(F)F)nc(C)c1S(=O)(=O)N1C[C@H]2CN(C3CC4(COC4)C3)C[C@@H]2C1]

2-Oxaspiro[3.3]heptan-6-one (34.78 mg, 310.22 umol) was added to a solution of (3aR,6aR)-2-((4,6-dimethyl-2-(trifluoromethyl)pyrimidin-5-yl)sulfonyl)octahydropyrrolo[3,4-c]pyrrole (60 mg, 155.11 umol, HCl salt) and TEA (31.39 mg, 310.22 umol, 43.24 uL) in MeOH (2 mL). The pH of the solution was adjusted to pH=5-6 by addition of HOAc. The reaction was stirred for 30 min, then NaBH3CN (19.49 mg, 310.22 umol) was added and the mixture was stirred at 20° C. for 2 h. The mixture was filtered and concentrated in vacuum. The crude was purified by prep-HPLC (Column:Welch Xtimate C18 150*25 mm*5 um; Condition: water (NH₄HCO₃)-ACN; Begin B: 32; End B: 60; Flow Rate (ml/min): 25) to give the desired compound (25 mg, 36.1% yield) as a white solid. LCMS m/z=447.1 [M-tBu+H]⁺. ¹HNMR (500 MHz, MeOD): δ ppm 4.70 (s, 2H), 4.60 (s, 2H), 3.64-3.61 (m, 2H), 3.26-3.23 (m, 1H), 3.22-3.20 (m, 2H), 2.92 (s, 6H), 2.91-2.87 (m, 2H), 2.46-2.42 (m, 4H), 2.31-2.28 (m, 2H), 2.09-2.07 (m, 2H).

### Example 62: (3aR,6aR)-2-((4,6-dimethyl-2-(trifluoromethyl)pyrimidin-5-yl)sulfonyl)-5-((tetrahydro-2H-pyran-4-yl)methyl)octahydropyrrolo[3,4-c]pyrrole

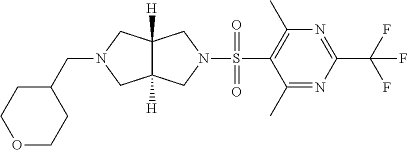 [SMILES: Cc1nc(C(F)(F)F)nc(C)c1S(=O)(=O)N1C[C@H]2CN(CC3CCOCC3)C[C@@H]2C1]

(3aR,6aR)-2-((4,6-dimethyl-2-(trifluoromethyl)pyrimidin-5-yl)sulfonyl)-5-((tetrahydro-2H-pyran-4-yl)methyl)octahydropyrrolo[3,4-c]pyrrole was obtained (21 mg, 30%) from (3aR,6aR)-2-((4,6-dimethyl-2-(trifluoromethyl)pyrimidin-5-yl)sulfonyl)octahydropyrrolo[3,4-c]pyrrole and tetrahydro-2H-pyran-4-carbaldehyde, following a similar procedure described in Example 61 step 3. LCMS m/z=449.1 [M+H]⁺. ¹HNMR (500 MHz, MeOD): δ ppm 3.94-3.91 (m, 2H), 3.64-3.61 (m, 2H), 3.42-3.37 (m, 2H), 3.10-3.05 (m, 2H), 2.93-2.88 (m, 8H), 2.60-2.55 (m, 4H), 2.36-2.34 (m, 2H), 1.71-1.68 (m, 3H), 1.25-1.19 (m, 2H).

### ASSAYS

### EBP Functional Assay

The EBP immunoaffinity (IA) LC-MS assay measures the potency of small molecule inhibitors of EBP by quantifying their concentration-dependent changes in the enzyme's substrate and product using liquid chromatography atmospheric pressure chemical ionization multiple reaction monitoring mass spectrometry (LC-APCI MRM MS). HEK293T cells were utilized as the source of EBP enzyme. The enzyme was incubated with the small molecule inhibitors at variable concentrations for 30 min. Deuterated form of EBP substrate, zymosterol-d5 (Avanti Polar Lipids, Cat #700068P-1 mg), was then added and the plate was incubated at 37° C. for 4 h. Finally, the sterol isomers were extracted and injected to LC-APCI MRM MS. MRM transition used for the quantification for both zymosterol and dihydrolathosterol (substrate and product of EBP enzymatic reaction, respectively) is 372.3-203.2, CE 30 and DP 80 in positive ion mode. Percent conversion of the zymosterol-d₅ to dehydrolathosterol-d₅ was used to derive IC₅₀ curves. Tasin-1 (1′-[(4-Methoxyphenyl)sulfonyl]-4-methyl-1,4′-bipiperidine, CAS 792927-06-1) was used as the reference small molecule inhibitor.

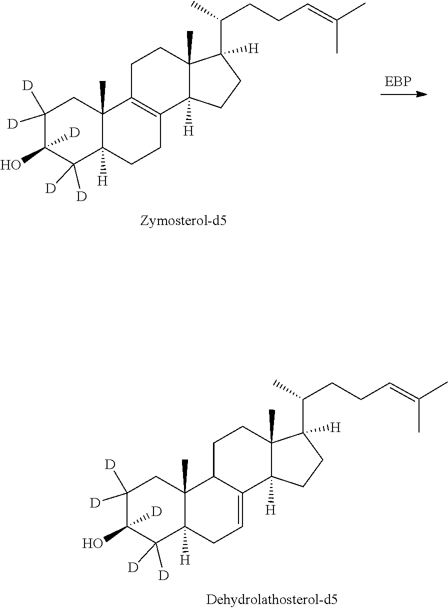

Percent conversion versus the compound concentration data were fit to the following 4-parameter logistic model to generate IC₅₀ curves:

$$y = LA + \frac{UA - LA}{1 + 1 {0}^{( log {IC}_{50} - x ) \cdot Slope}}$$

### Data for Examples

| Example Number | EBP Functional Assay |
|---|---|
| 1 | +++ |
| 2 | ++ |
| 3 | +++ |
| 4 | ++ |
| 5 | +++ |
| 6 | ++ |
| 7 | +++ |
| 8 | ++ |
| 9 | ++ |
| 10 | + |
| 11 | NT |
| 12 | ++ |
| 13 | + |
| 14 | + |
| 15 | ++ |
| 16 | +++ |
| 17 | ++ |
| 18 | NT |
| 19 | ++ |
| 20 | ++ |
| 21 | +++ |
| 22 | NT |
| 23 | +++ |
| 24 | NT |
| 25 | NT |
| 26 | NT |
| 27 | +++ |
| 28 | +++ |
| 29 | +++ |
| 30 | ++ |
| 31 | ++ |
| 32 | +++ |
| 33 | + |
| 34 | +++ |
| 35 | +++ |
| 36 | NT |
| 37 | NT |
| 38 | NT |
| 39 | ++ |
| 40 | NT |
| 41 | NT |
| 42 | NT |
| 43 | NT |
| 44 | NT |
| 45 | ++ |
| 46 | ++ |
| 47 | ++ |
| 48 | ++ |
| 49 | + |
| 50 | +++ |
| 51 | +++ |
| 52 | + |
| 53 | ++ |
| 54 | ++ |
| 55 | ++ |
| 56 | ++ |
| 57 | ++ |
| 58 | ++ |
| 59 | ++ |
| 60 | +++ |
| 61 | +++ |
| 62 | +++ |

*+ means >0.5 μM;
++ means 0.1-0.5 μM;
+++ means <0.1 μM;
NT = Not Tested

What is claimed is:

1. A compound represented by Formula (I): 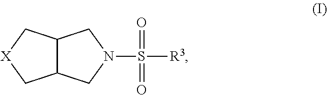
or a pharmaceutically acceptable salt thereof, wherein:
X is NR¹ or CR^{X};
R^{X} is —NR¹R²;
R¹ and R² are each independently selected from H, C₁₋₆alkyl, C₄₋₈cycloalkyl, Het, or —Z-Het, wherein the C₁₋₆alkyl, C₄₋₈cycloalkyl, and Het are each optionally substituted with one or more R⁴, provided at least one of R¹ and R² is not H;
or R¹ and R², together with the N atom from which they are attached, form a 4 to 7-membered monocyclic heterocycle or 6 to 10-membered bicyclic heterocycle, each of which is optionally substituted with one or more R⁴;
Z is C₁₋₄alkyl;
Het is a 4 to 6 membered monocyclic heterocyclyl, or a 6 to 8-membered bicyclic heterocyclyl, each of which is optionally substituted with one or more R⁴;
R⁴, for each occurrence, is independently C₁₋₆alkyl or halo;
R³ is phenyl, 5 or 6-membered monocyclic heteroaryl, 9 or 10-membered bicyclic heteroaryl, or 6 to 10 membered bicyclic heterocycle, wherein the phenyl, 5 or 6-membered monocyclic heteroaryl, 9 or 10-membered bicyclic heteroaryl, and 6 to 10 membered bicyclic heterocycle are each optionally substituted with one or more substituent R⁵;
R⁵, for each occurrence, is independently selected from halo, C₁₋₄alkyl, C₁₋₄ haloalkyl, C₃₋₈cycloalkyl, —OR^{5a}, and cyano;
R^{5a} is selected from H, C₁₋₄alkyl, and C₁₋₄haloalkyl;
provided that the compound is not the following compound:

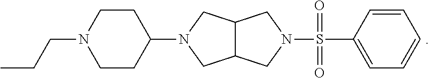 [SMILES: CCCN1CCC(N2CC3CN(S(=O)(=O)c4ccccc4)CC3C2)CC1]

2. The compound of claim 1, or a pharmaceutically acceptable salt thereof, wherein Het is a 4 to 6 membered oxygen-containing monocyclic saturated heterocyclyl or a 6 to 8-membered oxygen-containing bicyclic saturated heterocyclyl.

3. The compound of claim 1 or 2, or a pharmaceutically acceptable salt thereof, wherein R³ is phenyl, 5 or 6-membered monocyclic heteroaryl, 9 to 10 membered bicyclic heteroaryl or 8 to 10 membered bicyclic heterocycle, wherein the phenyl, 5 or 6-membered monocyclic heteroaryl, 9 to 10 membered bicyclic heteroaryl and 8 to 10 membered bicyclic heterocycle are each optionally substituted with one to three R⁵.

4. The compound of any one of claims 1 to 3, wherein the compound is represented by Formula (II): 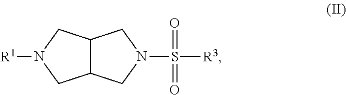
or a pharmaceutically acceptable salt thereof.

5. The compound of claim 4, or a pharmaceutically acceptable salt thereof, wherein R³ is selected from the group consisting of phenyl, pyridyl, pyrimidinyl, and pyrazolyl.

6. The compound of claim 5, or a pharmaceutically acceptable salt thereof, wherein R³ is represented by the following formula: 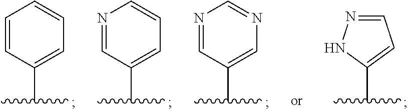
wherein each of the formula depicted above is optionally substituted with one to three R⁵.

7. The compound of claim 5, or a pharmaceutically acceptable salt thereof, wherein R³ is represented by the following formula:

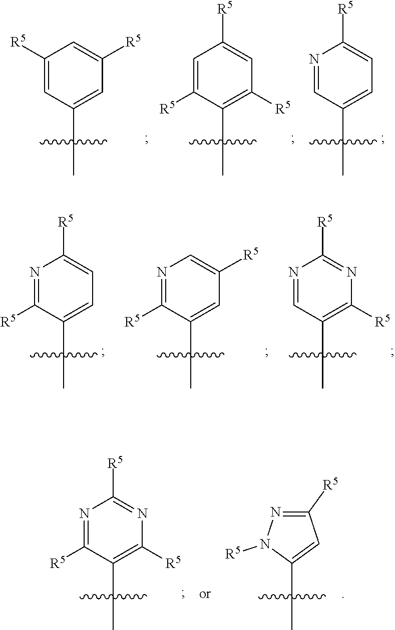

8. The compound of claim 5, or a pharmaceutically acceptable salt thereof, wherein R³ is represented by the following formula:

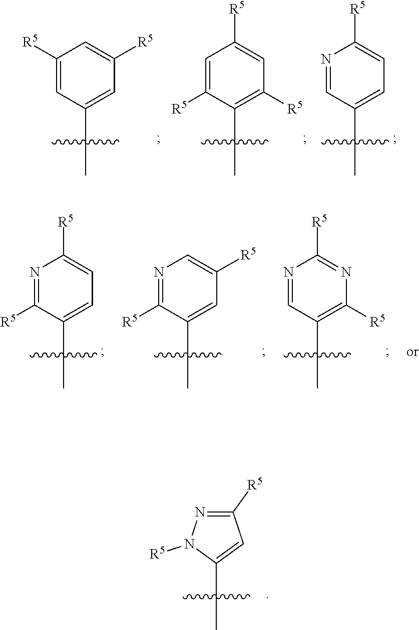

9. The compound of any one of claims 1 to 8, or a pharmaceutically acceptable salt thereof, wherein R⁵, for each occurrence, is independently selected from halo, C₁₋₄alkyl, C₁₋₄haloalkyl, —OR^{5a}, and —CN.

10. The compound of claim 9, or a pharmaceutically acceptable salt thereof, wherein R⁵, for each occurrence, is independently selected from —Cl, —F, —CH₃, —CF₃, —OCH₃, and —CN.

11. The compound of any one of claims 1 to 10, or a pharmaceutically acceptable salt thereof, wherein R¹ is —CH₂—Het or Het.

12. The compound of any one of claims 1 to 10, or a pharmaceutically acceptable salt thereof, wherein R¹ is Het.

13. The compound of claim 11, or a pharmaceutically acceptable salt thereof, wherein Het is selected from the group consisting of oxetanyl, 2-oxaspiro[3.3]heptanyl, tetrahydropyranyl and 2-oxabicyclo[2.1.1]hexanyl, 2-oxabicyclo[3.1.1]heptanyl, tetrahydrofuranyl.

14. The compound of claim 12, or a pharmaceutically acceptable salt thereof, wherein Het is selected from the group consisting of tetrahydropyranyl and tetrahydrofuranyl.

15. The compound of claim 13, or a pharmaceutically acceptable salt thereof, wherein Het is represented by the following formula: 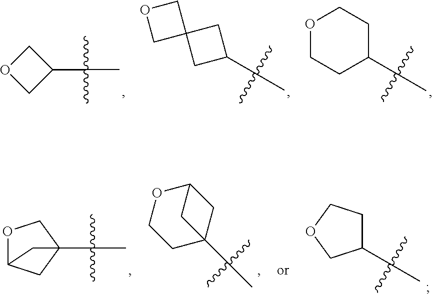 [SMILES: *C1COC1 ; *C1CC2(COC2)C1 ; *C1CCOCC1 ; *C12COC(C1)C2 ; *C12CCOC(C1)C2 ; *C1CCOC1]
wherein each of the formula depicted above is optionally substituted with one or two R⁴.

16. The compound of claim 14, or a pharmaceutically acceptable salt thereof, wherein Het is represented by the following formula: 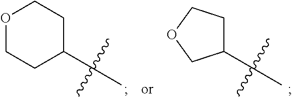 [SMILES: *C1CCOCC1 ; *C1CCOC1]
wherein each of the formula depicted above is optionally substituted with one or two R⁴.

17. The compound of claim 13, or a pharmaceutically acceptable salt thereof, wherein Het is represented by the following formula:

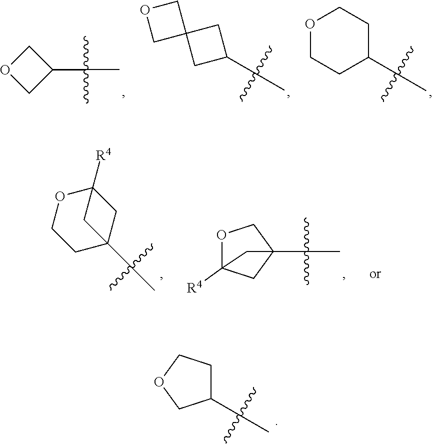

18. The compound of claim 14, or a pharmaceutically acceptable salt thereof, wherein Het is represented by the following formula:

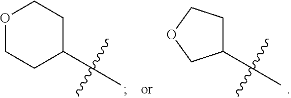 [SMILES: *C1CCOCC1 ; *C1CCOC1]

19. The compound of any one of claims 1 to 18, wherein the compound is represented by Formula (III): 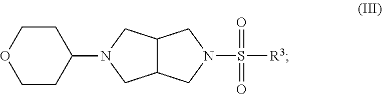
or a pharmaceutically acceptable salt thereof.

20. The compound of claim 19, or a pharmaceutically acceptable salt thereof, wherein R³ is represented by the following formula: 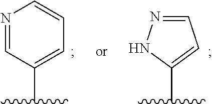 [SMILES: *c1cccnc1 ; *c1ccn[nH]1]
wherein each of the formula depicted above is optionally substituted with one to three R⁵.

21. The compound of claim 19, or a pharmaceutically acceptable salt thereof, wherein R³ is represented by the following formula:

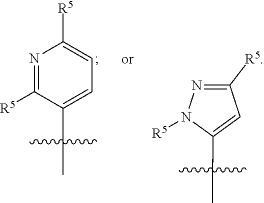

22. The compound of any one of claims 1 to 21, or a pharmaceutically acceptable salt thereof, wherein R⁵, for each occurrence, is independently selected from C₁₋₄alkyl and C₁₋₄haloalkyl.

23. The compound of claim 22, or a pharmaceutically acceptable salt thereof, wherein R⁵, for each occurrence, is independently selected from —CH₃, - and —CF₃.

24. The compound of any one of claim 1 to 3, wherein the compound is represented by Formula (IV): 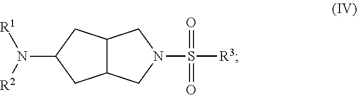
or a pharmaceutically acceptable salt thereof.

25. The compound of claim 24, or a pharmaceutically acceptable salt thereof, wherein R³ is selected from the group consisting of phenyl, pyridyl, and pyrazolyl.

26. The compound of claim 25, or a pharmaceutically acceptable-salt-thereof, wherein R³ is represented by the following formula: 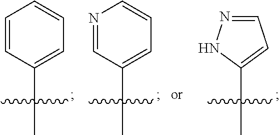
wherein each of the formula depicted above is optionally substituted with one to three R⁵.

27. The compound of claim 25, or a pharmaceutically acceptable salt thereof, wherein R³ is represented by the following formula:

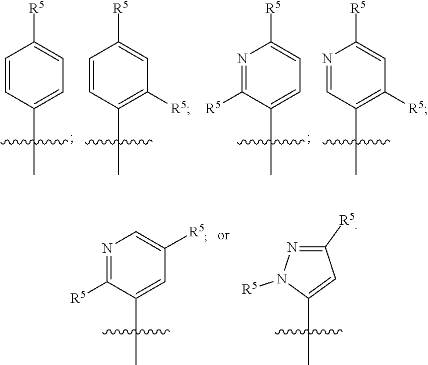

28. The compound of any one of claims 1 to 3 and 24 to 27, or a pharmaceutically acceptable salt thereof, wherein R⁵, for each occurrence, is independently selected from C₁₋₄alkyl, C₁₋₄haloalkyl, —OR^{5a}, and C₃₋₈cycloalkyl.

29. The compound of claim 28, or a pharmaceutically acceptable salt thereof, wherein R⁵, for each occurrence, is independently selected from —CH₃, —CF₃, —OCH₃, —OCHF₂, and cyclopropyl.

30. The compound of any one of claims 1 to 3, and 24 to 29, or a pharmaceutically acceptable salt thereof, wherein R¹ is Het or —Z-Het; and R² is H or C₁₋₆alkyl.

31. The compound of claim 30, or a pharmaceutically acceptable salt thereof, wherein Het is selected from the group consisting of tetrahydropyranyl, tetrahydrofuranyl, and 2-oxaspiro[3.3]heptanyl.

32. The compound of claim 31, or a pharmaceutically acceptable salt thereof, wherein R¹ is represented by the following formula: 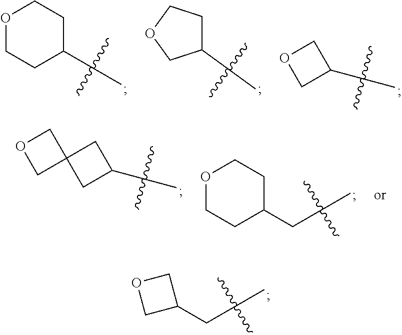 [SMILES: *C1CCOCC1 ; *C1CCOC1 ; *C1COC1 ; *C1CC2(COC2)C1 ; *CC1CCOCC1 ; *CC1COC1]
wherein each of the formula depicted above is optionally substituted with one or two R⁴.

33. The compound of claim 31, or a pharmaceutically acceptable salt thereof, wherein R¹ is represented by the following formula:

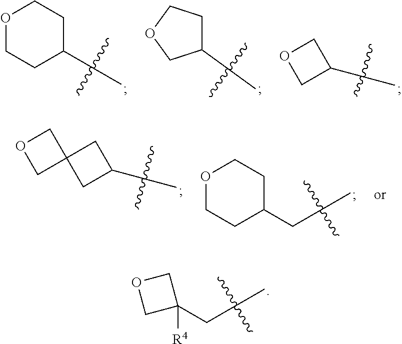

34. The compound of any one of claims 1 to 3 and 24 to 33, or a pharmaceutically acceptable salt thereof, wherein R² is H or —CH₃.

35. The compound of any one of claims 1 to 3 and 24 to 29, or a pharmaceutically acceptable salt thereof, wherein R¹ and R², together with the N atom from which they are attached, form a 4 to 7-membered monocyclic heterocycle optionally substituted with one or more R⁴.

36. The compound of claim 35, or a pharmaceutically acceptable salt thereof, wherein R¹ and R², together with the N atom from which they are attached are selected from the group consisting of piperdinyl and morpholinyl.

37. The compound of claim 36, or a pharmaceutically acceptable salt thereof, wherein R¹ and R², together with the N atom from which they are attached are represented by the following formula: 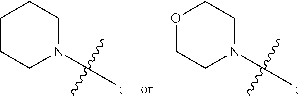 [SMILES: *N1CCCCC1 ; *N1CCOCC1]
wherein each of the formula depicted above is optionally substituted with one or two R⁴.

38. The compound of claim 36, or a pharmaceutically acceptable salt thereof, wherein R¹ and R², together with the N atom from which they are attached are represented by the following formula:

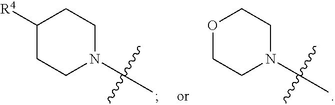

39. The compound of any one of claims 1 to 38, or a pharmaceutically acceptable salt thereof, wherein R⁴ is —CH₃.

40. The compound of claim 1, wherein the compound is represented by Formula (II): 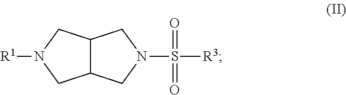
or a pharmaceutically acceptable salt thereof, wherein:
R¹ is Het, or —CH₂—Het
Het is a 4 to 6-membered monocyclic heterocyclyl, or a 6 to 8-membered bicyclic heterocyclyl, each of which is optionally substituted with one or more R⁴;
each R⁴ is independently C₁₋₃alkyl;
R³ is 5 or 6-membered monocyclic heteroaryl substituted with one or more R⁵;
each R⁵ is independently C₁₋₃alkyl and C₁₋₃haloalkyl.

41. The compound of claim 40, or a pharmaceutically acceptable salt thereof, wherein R³ is pyridinyl, pyrimidinyl, or pyrazoyl,

42. The compound of claim 41, or a pharmaceutically acceptable salt thereof, wherein R³ is represented by the following formula: 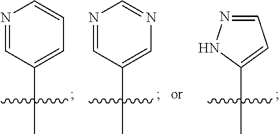
wherein each of the formula depicted above is optionally substituted with two or three R⁵.

43. The compound of claim 41, or a pharmaceutically acceptable salt thereof, wherein R³ is represented by the following formula:

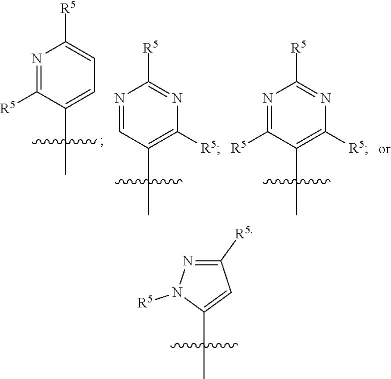

44. The compound of any one of claims 40 to 43, wherein each R⁵ is selected from —CF₃ and —CH₃.

45. The compound of any one of claims 40 to 44, or a pharmaceutically acceptable salt thereof, wherein Het is tetrohydropyranyl or 2-oxabicyclo[2.1.1]hexanyl.

46. The compound of claim 45, or a pharmaceutically acceptable salt thereof, wherein Het is represented by the following formula: 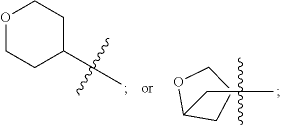 [SMILES: *C1CCOCC1 ; *CC1CCCO1]
wherein each of the formula depicted above is optionally substituted with one or two R⁴.

47. The compound of claim 45, or a pharmaceutically acceptable salt thereof, wherein Het is represented by the following formula:

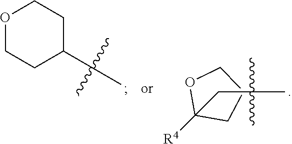

48. The compound of any one of claims 40 to 44, or a pharmaceutically acceptable salt thereof, wherein R¹ is represented by the following formula:

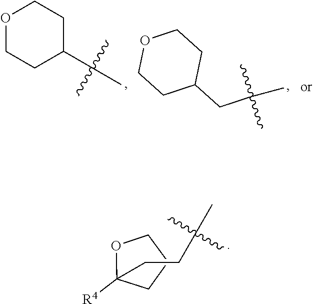

49. The compound of any one of claim 40 to 48, or a pharmaceutically acceptable salt thereof, wherein R⁴ is —CH₃.

50. The compound of claim 1, or a pharmaceutically acceptable salt thereof, wherein the compound is selected from:
(3aR,5r,6aS)-2-((2,4-Dimethylphenyl)sulfonyl)-N-((tetrahydro-2H-pyran-4-yl)methyl)octahydrocyclopenta[c]pyrrol-5-amine;
(3aR,5r,6aS)-2-((4-(Difluoromethoxy)phenyl)sulfonyl)-N-((tetrahydro-2H-pyran-4-yl)methyl)octahydrocyclopenta[c]pyrrol-5-amine;
(3aR,5s,6aS)-2-((2,4-Dimethylphenyl)sulfonyl)-5-(4-methylpiperidin-1-yl)octahydrocyclopenta[c]pyrrole;
(3aR,5s,6aS)-2-((4-(Difluoromethoxy)phenyl)sulfonyl)-5-(4-methylpiperidin-1-yl)octahydrocyclopenta[c]pyrrole;
(3aR,5r,6aS)-2-((2,4-Dimethylphenyl)sulfonyl)-5-(4-methylpiperidin-1-yl)octahydrocyclopenta[c]pyrrole;
(3aR,5r,6aS)-2-((4-(Difluoromethoxy)phenyl)sulfonyl)-5-(4-methylpiperidin-1-yl)octahydrocyclopenta[c]pyrrole;
(3aR,5s,6aS)-2-((2,4-Dimethylphenyl)sulfonyl)-N-((tetrahydro-2H-pyran-4-yl)methyl)octahydrocyclopenta[c]pyrrol-5-amine;
(3aR,5s,6aS)-2-((4-(Difluoromethoxy)phenyl)sulfonyl)-N-((tetrahydro-2H-pyran-4-yl)methyl)octahydrocyclopenta[c]pyrrol-5-amine;
(3aR,5s,6aS)-2-((2-Methoxy-5-methylpyridin-3-yl)sulfonyl)-N-((tetrahydro-2H-pyran-4-yl)methyl)octahydrocyclopenta[c]pyrrol-5-amine;
(3aR,5s,6aS)-2-((4,6-Dimethylpyridin-3-yl)sulfonyl)-N-((tetrahydro-2H-pyran-4-yl)methyl)octahydrocyclopenta[c]pyrrol-5-amine;
(3aR,5s,6aS)-2-((1,3-Dimethyl-1H-pyrazol-5-yl)sulfonyl)-N-((tetrahydro-2H-pyran-4-yl)methyl)octahydrocyclopenta[c]pyrrol-5-amine;
(3aR,5s,6aS)-2-((3-Cyclopropyl-1-methyl-1H-pyrazol-5-yl)sulfonyl)-N-((tetrahydro-2H-pyran-4-yl)methyl)octahydrocyclopenta[c]pyrrol-5-amine;
4-((3aR,5r,6aS)-2-((4-(Difluoromethoxy)phenyl)sulfonyl)octahydrocyclopenta[c]pyrrol-5-yl)morpholine;
(3aR,5s,6aS)-2-((4-(Difluoromethoxy)phenyl)sulfonyl)-N-(oxetan-3-yl)octahydrocyclopenta[c]pyrrol-5-amine;
(3aR,5s,6aS)-2-((4-(Difluoromethoxy)phenyl)sulfonyl)-N-(tetrahydrofuran-3-yl)octahydrocyclopenta[c]pyrrol-5-amine;
(3aR,5s,6aS)-2-((4-(Difluoromethoxy)phenyl)sulfonyl)-N-(tetrahydro-2H-pyran-4-yl)octahydrocyclopenta[c]pyrrol-5-amine;
(3aR,5s,6aS)-2-((4-(Difluoromethoxy)phenyl)sulfonyl)-N-methyl-N-((tetrahydro-2H-pyran-4-yl)methyl)octahydrocyclopenta[c]pyrrol-5-amine;
(3aR,5s,6aS)-2-((3-Cyclopropyl-1-methyl-1H-pyrazol-5-yl)sulfonyl)-N-methyl-N-((tetrahydro-2H-pyran-4-yl)methyl)octahydrocyclopenta[c]pyrrol-5-amine;
(3aR,5s,6aS)-2-((4-(Difluoromethoxy)phenyl)sulfonyl)-N-(2-oxaspiro[3.3]heptan-6-yl)octahydrocyclopenta[c]pyrrol-5-amine;
(3aR,5s,6aS)-2-((3-Cyclopropyl-1-methyl-1H-pyrazol-5-yl)sulfonyl)-N-(2-oxaspiro[3.3]heptan-6-yl)octahydrocyclopenta[c]pyrrol-5-amine;
(3aR,5s,6aS)-2-((3-Cyclopropyl-1-methyl-1H-pyrazol-5-yl)sulfonyl)-N-((3-methyloxetan-3-yl)methyl)octahydrocyclopenta[c]pyrrol-5-amine;
(3aR,5s,6aS)-2-((3-Cyclopropyl-1-methyl-1H-pyrazol-5-yl)sulfonyl)-N-(tetrahydro-2H-pyran-4-yl)octahydrocyclopenta[c]pyrrol-5-amine;
(3aR,5s,6aS)-N-Methyl-2-((2-methyl-6-(trifluoromethyl)pyridin-3-yl)sulfonyl)-N-((tetrahydro-2H-pyran-4-yl)methyl)octahydrocyclopenta[c]pyrrol-5-amine;
(3aR,5s,6aS)-N-Methyl-2-((2-methyl-6-(trifluoromethyl)pyridin-3-yl)sulfonyl)-N-(tetrahydro-2H-pyran-4-yl)octahydrocyclopenta[c]pyrrol-5-amine;
(3aR,5s,6aS)-N-Methyl-2-((2-methyl-6-(trifluoromethyl)pyridin-3-yl)sulfonyl)-N-(2-oxaspiro[3.3]heptan-6-yl)octahydrocyclopenta[c]pyrrol-5-amine;
(3aR,5s,6aS)-N-Methyl-2-((2-methyl-6-(trifluoromethyl)pyridin-3-yl)sulfonyl)-N-((3-methyloxetan-3-yl)methyl)octahydrocyclopenta[c]pyrrol-5-amine;
(3aR,5s,6aS)-2-((2-Methyl-6-(trifluoromethyl)pyridin-3-yl)sulfonyl)-N-((tetrahydro-2H-pyran-4-yl)methyl)octahydrocyclopenta[c]pyrrol-5-amine;
(3aR,5s,6aS)-2-((2-Methyl-6-(trifluoromethyl)pyridin-3-yl)sulfonyl)-N-(tetrahydro-2H-pyran-4-yl)octahydrocyclopenta[c]pyrrol-5-amine;
(3aR,5s,6aS)-2-((2-Methyl-6-(trifluoromethyl)pyridin-3-yl)sulfonyl)-N-((3-methyloxetan-3-yl)methyl)octahydrocyclopenta[c]pyrrol-5-amine;
(3aR,5s,6aS)-2-((2-Methyl-6-(trifluoromethyl)pyridin-3-yl)sulfonyl)-N-(2-oxaspiro[3.3]heptan-6-yl)octahydrocyclopenta[c]pyrrol-5-amine;
(3aR,6aS)-2-((1,3-dimethyl-1H-pyrazol-5-yl)sulfonyl)-5-(tetrahydro-2H-pyran-4-yl)octahydropyrrolo[3,4-c]pyrrole;
2-(Mesitylsulfonyl)-5-(tetrahydro-2H-pyran-4-yl)octahydropyrrolo[3,4-c]pyrrole;
3-Fluoro-5-(((3aR,6aS)-5-(tetrahydro-2H-pyran-4-yl)hexahydropyrrolo[3,4-c]pyrrol-2(1H)-yl)sulfonyl)benzonitrile;
(3aR,6aS)-2-((6-Methoxy-2-methylpyridin-3-yl)sulfonyl)-5-(tetrahydro-2H-pyran-4-yl)octahydropyrrolo[3,4-c]pyrrole;
(3aR,6aS)-2-((2-Methyl-6-(trifluoromethyl)pyridin-3-yl)sulfonyl)-5-(tetrahydro-2H-pyran-4-yl)octahydropyrrolo[3,4-c]pyrrole;
(3aR,6aS)-2-((1-Methyl-3-(trifluoromethyl)-1H-pyrazol-5-yl)sulfonyl)-5-(tetrahydro-2H-pyran-4-yl)octahydropyrrolo[3,4-c]pyrrole;
(3aR,6aS)-2-((4-Methyl-2-(trifluoromethyl)pyrimidin-5-yl)sulfonyl)-5-(tetrahydro-2H-pyran-4-yl)octahydropyrrolo[3,4-c]pyrrole;
(3aR,6aS)-2-((2-Chloro-6-methoxypyridin-3-yl)sulfonyl)-5-(tetrahydro-2H-pyran-4-yl)octahydropyrrolo[3,4-c]pyrrole;
(3aR,6aS)-2-((3,5-Difluorophenyl)sulfonyl)-5-(tetrahydro-2H-pyran-4-yl)octahydropyrrolo[3,4-c]pyrrole;
(3aR,6aS)-2-((6-Methoxypyridin-3-yl)sulfonyl)-5-(tetrahydro-2H-pyran-4-yl)octahydropyrrolo[3,4-c]pyrrole;
(3aR,6aS)-2-((5-Chloro-2-methoxypyridin-3-yl)sulfonyl)-5-(tetrahydro-2H-pyran-4-yl)octahydropyrrolo[3,4-c]pyrrole;
(3aR,6aS)-2-((2-Methoxy-5-methylpyridin-3-yl)sulfonyl)-5-(tetrahydro-2H-pyran-4-yl)octahydropyrrolo[3,4-c]pyrrole;
(3aR,6aS)-2-((2,4-Dimethylpyrimidin-5-yl)sulfonyl)-5-(tetrahydro-2H-pyran-4-yl)octahydropyrrolo[3,4-c]pyrrole;
(3aR,6aS)-2-((2,4-Dimethylpyrimidin-5-yl)sulfonyl)-5-(tetrahydrofuran-3-yl)octahydropyrrolo[3,4-c]pyrrole;
(3aR,6aR)-2-((2-Methyl-6-(trifluoromethyl)pyridin-3-yl)sulfonyl)-5-(tetrahydro-2H-pyran-4-yl)octahydropyrrolo[3,4-c]pyrrole;
(3aR,6aR)-2-((1-Methyl-3-(trifluoromethyl)-1H-pyrazol-5-yl)sulfonyl)-5-(tetrahydro-2H-pyran-4-yl)octahydropyrrolo[3,4-c]pyrrole;
(3aS,6aS)-5-[4,6-dimethyl-2-(trifluoromethyl)pyrimidin-5-yl]sulfonyl-2-(oxan-4-ylmethyl)-1,3,3a,4,6,6a-hexahydropyrrolo[3,4-c]pyrrole;
(3aS,6aS)-5-[4,6-dimethyl-2-(trifluoromethyl)pyrimidin-5-yl]sulfonyl-2-(2-oxaspiro[3.3]heptan-6-yl)-1,3,3a,4,6,6a-hexahydropyrrolo[3,4-c]pyrrole;
(3aS,6aS)-5-[4,6-dimethyl-2-(trifluoromethyl)pyrimidin-5-yl]sulfonyl-2-(oxan-4-yl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(2-oxaspiro[3.3]heptan-6-yl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(oxan-4-ylmethyl)-1,3,3a,4,6,6a-hexahydropyrrolo[3,4-c]pyrrole;
rac-(3aR,6aR)-5-[4-methyl-2-(trifluoromethyl)pyrimidin-5-yl]sulfonyl-2-(2-oxaspiro[3.3]heptan-6-yl)-1,3,3a,4,6,6a-hexahydropyrrolo[3,4-c]pyrrole;
rac-(3aR,6aR)-5-[4-methyl-2-(trifluoromethyl)pyrimidin-5-yl]sulfonyl-2-(oxan-4-ylmethyl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(oxan-4-yl)-1,3,3a,4,6,6a-hexahydropyrrolo[3,4-c]pyrrole;
(3aS,6aS)-2-[(1-methyl-2-oxabicyclo[2.1.1]hexan-4-yl)methyl]-5-[2-methyl-6-(trifluoromethyl)pyridin-3-yl]sulfonyl-1,3,3a,4,6,6a-hexahydropyrrolo[3,4-c]pyrrole;
rel-(3aR,6aR)-2-[(1-methyl-2-oxabicyclo[3.1.1]heptan-5-yl)methyl]-5-[2-methyl-6-(trifluoromethyl)pyridin-3-yl]sulfonyl-1,3,3a,4,6,6a-hexahydropyrrolo[3,4-c]pyrrole;
(3aR,6aR)-2-[(1-methyl-2-oxabicyclo[2.1.1]hexan-4-yl)methyl]-5-[2-methyl-6-(trifluoromethyl)pyridin-3-yl]sulfonyl-1,3,3a,4,6,6a-hexahydropyrrolo[3,4-c]pyrrole;
(3aS,6aS)-2-[(1-methyl-2-oxabicyclo[2.1.1]hexan-4-yl)methyl]-5-[2-methyl-6-(trifluoromethyl)pyridin-3-yl]sulfonyl-1,3,3a,4,6,6a-hexahydropyrrolo[3,4-c]pyrrole;
rac-(3aR,6aR)-5-[4-methyl-2-(trifluoromethyl)pyrimidin-5-yl]sulfonyl-2-(oxan-4-yl)-1,3,3a,4,6,6a-hexahydropyrrolo[3,4-c]pyrrole;
rac-(3aR,6aR)-5-[2-methyl-6-(trifluoromethyl)pyridin-3-yl]sulfonyl-2-(oxan-4-yl)-1,3,3a,4,6,6a-hexahydropyrrolo[3,4-c]pyrrole;
(3aR,6aRS)-5-(2,5-dimethylpyrazol-3-yl)sulfonyl-2-(oxan-4-yl)-1,3,3a,4,6,6a-hexahydropyrrolo[3,4-c]pyrrole; and
rac-(3aR,6aR)-5-[2-methyl-6-(trifluoromethyl)pyridin-3-yl]sulfonyl-2-(oxetan-3-ylmethyl)-1,3,3a,4,6,6a-hexahydropyrrolo[3,4-c]pyrrole;
or a pharmaceutically acceptable salt thereof.

51. A pharmaceutical composition comprising a compound of any one of claims 1 to 50, or a pharmaceutically acceptable salt thereof, and a pharmaceutically acceptable excipient.

52. A method of treating a disease mediated by Emopamil-Binding Protein comprising administering to a subject an effective amount of a compound of any one of claims 1 to 50, or a pharmaceutically acceptable salt thereof, or the pharmaceutical composition of claim 51.

53. A method of treating an autoimmune disease in a subject comprising administering to the subject an effective amount of a compound of any one of claims 1 to 50, or a pharmaceutically acceptable salt thereof, or the pharmaceutical composition of claim 51.

54. The method of claim 53, wherein said autoimmune disease is multiple sclerosis.

55. The method of claim 54, wherein said compound or pharmaceutical composition repairs or forms new myelin sheaths in said subject.
